(19) United States
(12) Patent Application Publication
(10) Pub. No.: US 2009/0239794 A1
(43) Pub. Date: Sep. 24, 2009

(54) HCV F PROTEIN AND USES THEREOF

(75) Inventors: Hugo Soudeyns, Laval (CA); Myriam Troesch, Montreal (CA); Emilie Jalbert, Seattle, WA (US)

Correspondence Address: FULBRIGHT & JAWORSKI L.L.P., 600 CONGRESS AVE., SUITE 2400, AUSTIN, TX, 78701, US

(73) Assignee: Valorisation HSJ, Societe en Commandite, Montreal (CA)

(21) Appl. No.: 11/719,635

(22) PCT Filed: Nov. 18, 2005
(86) PCT No.: PCT/CA2005/001756

§ 371 Date: Jun. 11, 2009

Related U.S. Application Data
(60) Provisional application No. 60/628,549, filed on Nov. 18, 2004.

(51) Int. Cl. A61K 38/16; C07K 14/00; C07H 21/02; C12N 15/74; C12N 5/10; C12P 21/00; G01N 33/53; A61P 37/04

(52) U.S. Cl. 514/12; 530/324; 536/23.1; 435/320.1; 435/325; 435/69.1; 435/7.1

(57) ABSTRACT

The invention provides polypeptides, nucleic acids, antibodies, compositions, vaccines, microarrays and uses thereof for the prevention, treatment of HCV infection. The invention further provides uses of the above-noted products for the detection and diagnosis of HCV infection. The invention further provides corresponding methods and commercial packages relating to such uses. The invention further provides recombinant polypeptides and methods for their production.

### CROSS REFERENCE TO RELATED APPLICATIONS

This application is a national phase application under 35 U.S.C. § 371 of International Application No. PCT/CA2005/001756, filed Nov. 18, 2005, which claims the benefit, under 35 U.S.C. § 119(e), of U.S. provisional patent application Ser. No. 60/628,549 filed Nov. 18, 2004. All of these applications are incorporated herein by reference in their entirety.

### FIELD OF THE INVENTION

The invention relates to peptides and corresponding nucleic acids and uses thereof for prevention, treatment and diagnosis of hepatitis C virus (HCV) infection, and particularly relates to peptides and corresponding nucleic acids derived from the HCV F protein, methods of preparation thereof, and uses thereof for prevention, treatment and diagnosis of HCV infection.

### BACKGROUND OF THE INVENTION

Cell-mediated immune responses comprised of CD4+ T helper (Th) and CD8+ cytotoxic T lymphocytes (CTL) constitute the main mechanisms by which HCV replication can be controlled in the host [1-4]. Due to overlapping transmission routes and target populations, a large proportion of chronic hepatitis C virus (HCV) carriers are also infected with human immunodeficiency virus type I (HIV-1), which markedly worsens the prognosis of HCV disease in adults [5,6]. This is thought to result at least in part from inhibition of HCV-specific humoral and cell-mediated immunity by HIV-1 [7-9]. Cryptic epitopes generated from translation of viral or cellular gene products using alternate reading frames are known to elicit such responses and were recently shown to represent an important source of tumour-specific antigens in certain forms of human cancer [10,11]. Various RNA and DNA viruses of bacteria, plants and animals use overlapping open reading frames (ORF) to compensate for size limitations imposed on viral genomes and/or to regulate viral protein expression. Likewise, HCV encodes an alternate 144-162 amino-acid, 17 kDa polypeptide of unknown function termed F protein or alternate reading frame protein (ARFP) from the 5′ moiety of the core gene [12-14]. F protein is expressed in transfected cells [15] and can be recognized by sera and T cells isolated from HCV-infected patients, suggesting that it is produced in vivo [13, 14, 16, 17].

Given the prevalence and adverse consequences of HCV infection and related disease, there is therefore a need to develop methods and agents for the prevention, treatment and diagnosis of HCV infection.

### SUMMARY OF THE INVENTION

The invention relates to epitopes derived from HCV F protein and uses thereof.

“HCV F protein” is also referred to in the art as “alternate reading frame protein (ARFP)” [14]. Both of these terms are equivalent and are used interchangeably herein.

In a first aspect, the invention provides an isolated immunogenic polypeptide derived from the HCV F protein, with the proviso that said polypeptide is not the full length HCV F protein set forth in SEQ ID NO: 8.

In an embodiment, the HCV F protein is derived from an HCV of a subtype other than HCV-1b. In a further embodiment HCV F protein is derived from an HCV of a subtype selected from the group consisting of HCV-1a, HCV-2a, HCV-3a, HCV-4-a, HCV-5a and HCV-6a.

In an embodiment, the polypeptide is defined by the start and end positions within HCV F protein as defined in Table 16.

In embodiments, the polypeptide or epitope of the invention comprises an amino acid sequence having the following general formula I (see Table 14):

X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³

wherein;
X¹ is selected from V, A and D or is absent;
X² is R or is absent;
X³ is S or is absent;
X⁴ is L or is absent;
X⁵ is selected from V and A or is absent;
X⁶ is E or is absent;
X⁷ is selected from F and Y or is absent;
X⁸ is T or is absent;

### X⁹ is C;

### X¹⁰ is C;

### X¹¹ is R;

### X¹² is A;

X¹³ is selected from G and R or is absent;
X¹⁴ is A or is absent;
X¹⁵ is selected from L, P and H or is absent;
X¹⁶ is selected from D, G and N or is absent;
X¹⁷ is W or is absent;
X¹⁸ is V or is absent;
X¹⁹ is selected from C and S or is absent;
X²⁰ is A or is absent;
X²¹ is R or is absent;
X²² is selected from R, Q and L or is absent; and
X²³ is selected from E, G and V or is absent.

In further embodiments, the polypeptide or epitope of the invention comprises an amino acid sequence having the following general formula II (see Table 15):

Z¹-Z²-Z³-Z⁴-Z⁵-Z⁶-Z⁷-Z⁸-Z⁹-Z¹⁰-Z¹¹-Z¹²-Z¹³-Z¹⁴-Z¹⁵

wherein;
Z¹ is selected from P and H or is absent;
Z² is selected from V, E and E or is absent;
Z³ is selected from V and A or is absent;
Z⁴ is selected from L and P or is absent;
Z⁵ is selected from G, V and D or is absent;
Z⁶ is selected from L, P, H and R or is absent;
Z⁷ is selected from A, L, P, I, T and V or is absent;

### Z⁸ is G;

### Z⁹ is A;

Z¹⁰ is selected from P and Q;
Z¹¹ is selected from Q and M;

### Z¹² is T;

### Z¹³ is P;

Z¹⁴ is selected from G and A;
Z¹⁵ is selected from V, I and G.

In an embodiment, the peptide is selected from: (a) a polypeptide comprising an amino acid sequence selected from SEQ ID NOs: 9, 11, 12, 14 to 38, 51 to 103 and/or 122 to 128; and/or (b) a functional variant or fragment of (a), wherein said functional variant or fragment has an immune-related activity.

In an embodiment, the immune-related activity is selected from: (i) an induction of an immune response against HCV; (ii) an induction of T-cell lytic activity; (iii) binding to a human leukocyte antigen (HLA) or MHC class I molecule; (iv) immunoreactivity with serum from an HCV-infected subject; (v) an alteration in cytokine or chemokine expression or production; and (vi) any combination of (i) to (v). In an embodiment, the HLA molecule is an HLA-A molecule (e.g. an HLA-A*0201 molecule).

In an embodiment, the polypeptide is 50 amino acids or less in length.

In an embodiment, the polypeptide consists essentially of an amino acid sequence selected from SEQ ID NOs: 9, 11, 12, 14 to 38, 51 to 103 and/or 122 to 128.

In an embodiment, the polypeptide is recombinant.

In a further aspect, the invention provides a preparation comprising the above-mentioned polypeptide, wherein said preparation is substantially free of an HCV protein other than the HCV F protein. In an embodiment, the HCV protein other than the HCV F protein is an HCV core protein.

In a further aspect, the invention provides an isolated HCV F protein peptide epitope, wherein said peptide epitope has an immune-related activity, with the proviso that said peptide epitope is not the full length HCV F protein set forth in SEQ ID NO: 8.

In an embodiment, the immune-related activity is selected from: (i) an induction of T-cell lytic activity; (ii) binding to a human leukocyte antigen (HLA) or MHC class I molecule; (iii) immunoreactivity with serum from an HCV-infected subject; (iv) an alteration in cytokine or chemokine expression or production; and (v) any combination of (i) to (iv).

In embodiments, the isolated peptide epitope consists essentially of about 8 to about 50 contiguous amino acids of an amino acid sequence selected from SEQ ID NOs: 9, 11, 12 and/or 122 to 128, in further embodiments, of about 8 to about 15 contiguous amino acids of an amino acid sequence selected from SEQ ID NOs: 9, 11, 12 and/or 122 to 128, in further embodiments, 9, 10 or 15 contiguous amino acids of an amino acid sequence selected from SEQ ID NOs: 9, 11, 12 and/or 122 to 128.

In further embodiments, the isolated peptide epitope comprises an amino acid sequence selected from SEQ ID NOs: 9, 11, 12, 14 to 38, 51 to 103 and/or 122 to 128. In a further embodiment, the isolated peptide epitope comprises 1-15 amino acid additions, in a further embodiment 1-30 amino acid additions, to an amino acid sequence selected from SEQ ID NOs: 9, 11, 12, 14 to 38, 51 to 103 and/or 122 to 128. In a further embodiment, the isolated peptide epitope consists essentially of an amino acid sequence selected from SEQ ID NOs: 9, 11, 12, 14 to 38, 51 to 103 and/or 122 to 128.

In a further aspect, the invention provides a pharmaceutical composition comprising the above-mentioned isolated polypeptide or peptide epitope and a pharmaceutically acceptable carrier. In an embodiment, the composition further comprises an adjuvant. In an embodiment, the composition further comprises an MHC molecule.

In a further aspect, the invention provides a composition comprising the above-mentioned isolated polypeptide or peptide epitope and an adjuvant.

In a further aspect, the invention provides a composition comprising the above-mentioned isolated polypeptide or peptide epitope and an MHC molecule.

In a further aspect, the invention provides a composition comprising a multimer of two or more MHC peptide complex monomers, each of said monomers comprising the above-mentioned polypeptide or peptide epitope and an MHC molecule.

In an embodiment, the above-mentioned MHC molecule comprises an MHC class I heavy chain or fusion protein thereof and a β2 microglobulin or fusion protein thereof.

In embodiments, the monomers are joined together into said multimer by virtue of a multivalent entity. In embodiments, the monomers are associated with said multivalent entity by virtue of an interaction chosen from biotin-avidin interactions, biotin-streptavidin interactions, coiled-coil domain interactions, and liposome-monomer cross-linking.

In an embodiment, the above-mentioned composition further comprises a second polypeptide different from said isolated polypeptide, wherein said second polypeptide is capable of inducing a HCV immune response. In an embodiment, the second polypeptide is an additional HCV polypeptide.

In a further aspect, the invention provides an antibody capable of specifically binding to the above-mentioned polypeptide and/or peptide epitope.

In a further aspect, the invention provides an isolated nucleic acid encoding the above-mentioned polypeptide and/or peptide epitope. In an embodiment, the nucleic acid comprises a fragment of a nucleotide sequence capable of encoding an amino acid sequence selected from SEQ ID NOs: 9, 11, 12 and/or 122 to 128. In an embodiment, the nucleic acid comprises a nucleotide sequence capable of encoding an amino acid sequence selected from SEQ ID NOs: 9, 11, 12 and/or 122 to 128. In an embodiment, the nucleic acid comprises the nucleotide sequence of SEQ ID NO: 10. In an embodiment, the nucleic acid comprises a nucleotide sequence capable of encoding an amino acid sequence selected from SEQ ID NOs: 9, 11, 12, 14 to 38, 51 to 103 and/or 122 to 128.

In a further aspect, the invention provides a vector comprising the above-mentioned nucleic acid operably-linked to a transcriptional regulatory sequence.

In a further aspect, the invention provides a host cell transformed or transfected with the above-mentioned vector.

In a further aspect, the invention provides a method of producing the above mentioned polypeptide or peptide epitope, the method comprising culturing the above-mentioned host cell under conditions permitting expression of said polypeptide or peptide epitope. In an embodiment, the method results in no or substantially no production of an HCV protein other than the HCV F protein (e.g. an HCV core protein). In an embodiment, the method further comprises recovering said polypeptide or peptide epitope produced.

In a further aspect, the invention provides a method of preventing or treating HCV infection, or for inducing an immunological or protective immune response against HCV, in an animal, said method comprising administering to said animal an agent selected from the above-mentioned polypeptide, preparation, peptide epitope, pharmaceutical composition and/or vector.

In an embodiment, the animal is a mammal, in a further embodiment, a human. In a further embodiment, the human further has an HIV infection.

In a further aspect, the invention provides a use of the above-mentioned polypeptide, preparation, peptide epitope, composition or vector for the preparation of a medicament.

In a further aspect, the invention provides a use of an agent selected from the above-mentioned polypeptide, preparation, peptide epitope, composition and/or vector, for preventing or treating HCV infection, or for inducing an immunological or protective immune response against HCV.

In a further aspect, the invention provides a use of an agent selected from the above-mentioned polypeptide, preparation, peptide epitope, composition and/or vector, for the preparation of a medicament for preventing or treating HCV infection, or for inducing an immunological or protective immune response against HCV.

In a further aspect, the invention provides a commercial package comprising an agent selected from the above-mentioned polypeptide, preparation, peptide epitope, pharmaceutical composition and/or vector, together with instructions for preventing or treating HCV infection, or for inducing an immunological or protective immune response against HCV.

In a further aspect, the invention provides a method of detecting or diagnosing HCV infection in an animal, said method comprising assaying a biological sample of said animal with the above-mentioned polypeptide, preparation, peptide epitope, or any combination thereof.

In a further aspect, the invention provides a method of detecting or quantifying-HCV in a biological sample of an animal, said method comprising assaying said biological sample with the above-mentioned polypeptide, preparation, peptide epitope, or any combination thereof.

In a further aspect, the invention provides a method of detecting or diagnosing HCV infection in an animal, said method comprising: contacting a biological sample of said animal with the above-mentioned polypeptide, preparation, peptide epitope, or any combination thereof; and determining the binding of a constituent of the biological sample to said polypeptide or peptide epitope; wherein said binding is indicative of HCV infection.

In a further aspect, the invention provides a method for detecting or quantifying HCV in a biological sample of an animal, said method comprising: (a) contacting said biological sample with the above-mentioned polypeptide, preparation, peptide epitope, or any combination thereof; and (b) determining the binding of a constituent of the biological sample to said polypeptide or peptide epitope; wherein said binding is indicative of the presence or quantity of HCV in said sample.

In a further aspect, the invention provides a method of detecting or diagnosing HCV infection in an animal, said method comprising assaying a biological sample of said animal with the above-mentioned antibody or composition.

In a further aspect, the invention provides a method for detecting or quantifying HCV in a biological sample of an animal, said method comprising assaying said biological sample with the above-mentioned antibody or composition.

In a further aspect, the invention provides a use of the above-mentioned polypeptide, preparation, peptide epitope, or any combination thereof, for in vitro diagnosis, detection or quantitation of HCV in a biological sample.

In a further aspect, the invention provides a use of a complex comprising the above-mentioned polypeptide or peptide epitope, and an MHC molecule, for labelling, detecting or isolating T-cells.

In a further aspect, the invention provides a use of a complex comprising the above-mentioned polypeptide or peptide epitope, and an MHC molecule, for detecting, selecting, sorting, or identifying T cell epitopes and/or amino acid sequences.

In an embodiment, the above-mentioned composition is an immunogenic or vaccine composition.

In a further aspect, the invention provides a polypeptide microarray comprising the above-mentioned polypeptide, peptide epitope, or both, bound to a substrate.

In an embodiment the polypeptide microarray further comprises an MHC molecule bound to said substrate.

In a further aspect, the invention provides a polypeptide microarray comprising the above-mentioned antibody.

In a further aspect, the invention provides a polypeptide microarray comprising an MHC complex bound to a substrate, said complex comprising the above-mentioned polypeptide or peptide epitope and an MHC molecule.

In a further aspect, the invention provides a method of detecting or diagnosing HCV infection in an animal, said method comprising: contacting a biological sample of said animal with the above-mentioned polypeptide microarray; and determining the binding of a constituent of the biological sample to said polypeptide microarray; wherein said binding is indicative of HCV infection.

In a further aspect, the invention provides a commercial package comprising a component selected from the above-mentioned polypeptide, peptide epitope, antibody and/or polypeptide microarray, together with instructions for detecting or diagnosing HCV infection.

In an embodiment, the above-mentioned biological sample is a tissue or body fluid of an animal.

### BRIEF DESCRIPTION OF THE DRAWINGS

FIG. 1. Expression and production of recombinant HCV F protein. A. Structure of the Fmut8 F protein expression cassette. Initiation sites and putative A-rich ribosomal frameshift sequences are boxed. Site-specific mutations introduced to switch and lock the translational reading frame are underlined. B. Purification of recombinant HCV F protein by nickel chelation chromatography. pLys or BL21 competent E. coli cells (Novagen) were transformed with empty pET-30c (vector) or with pET-30cFmut8. Extraction was either with 6M guanidium hydrochloride or 8M urea. C. Identification of Fmut8 by mass spectrometry. Individual peptide matches are displayed under the full-length Fmut8 sequence.

FIG. 2. F protein-specific antibody responses in HCV-infected subjects and patients coinfected with HIV-1. Ig responses were detected using Fmut8Δ11 ELISA, as described under Materials and Methods. HCV genotype was determined as previously described [18]. Open triangle: uninfected subject; open circles: HCV-1a; closed circles: HCV-1b; Open squares: HCV-3a; closed squares: HCV-4-a; open diamonds: HCV-4-c; closed diamonds: HCV-5a. The hatched line corresponds to the detection threshold.

FIG. 3. Precursor frequencies of HCV F protein specific CTL in HCV-infected subjects and patients coinfected with HCV and HIV-1. CTL activity was detected in standard ⁵¹Cr-release assays using autologous B lymphoblastoid cell lines and vaccinia recombinants expressing either HCV-1a F protein or HCV-1a p 364-1618 [23]. Limiting dilution analysis was performed as described under Materials and Methods. Solid lines and open squares: F protein-specific activity; dashed lines and open circles: p 364-1618.

FIG. 4. MHC-F protein peptide interaction. Binding of peptides derived from the F protein sequence to HLA-A*0201 was tested using the T2 binding assay, as described in the Examples. Testing of the F protein overlapping peptide panel (200 μg/ml). The hatched line represents the threshold level corresponding to the no peptide control.

FIG. 5. MHC-F protein peptide interaction. Binding of peptides derived from the F protein sequence to HLA-A*0201 was tested using the T2 binding assay, as described in the Examples. Dose-response analysis with selected candidate peptides. Open circles: 30 μg/ml; open squares: 100 μg/ml; open triangles: 200 μg/ml. “F” peptide nomenclature refers to peptides defined by indicated positions (e.g. 29-43) in SEQ ID NO: 11.

FIG. 6. Results of ARFP immunization studies in mice as per the Examples below. Total anti-ARFP immunoglobulin responses measured by ELISA in mice immunized with ARFP. A. Protocol 1. Open triangles: mice immunized with ARFP; open squares: mice immunized with phosphate-buffered saline (PBS). Dashed line indicates ELISA detection threshold. B. Protocol 2. Open triangles: mice immunized with ARFP; open squares: mice immunized with PBS. Dashed line indicates ELISA detection threshold. C. Kaplan-Meier analysis of protocols 1 and 2 using acquisition of ELISA titer above 1600 as outcome.

### DETAILED DESCRIPTION OF THE INVENTION

HCV F protein is encoded in an alternate reading frame overlapping the core protein region. This study was conducted to examine the prevalence and characteristics of host humoral and cell-mediated immune responses directed against F protein in patients coinfected with HCV and HIV-1.

In the studies described herein, mutations were introduced to allow expression of HCV-1a F protein in absence of core. This recombinant protein and a truncated form lacking the first 11 amino acid residues shared with core were expressed in E. coli and their amino acid sequences were verified by mass spectrometry. Vaccinia-F protein recombinants were used to test F protein-specific cytotoxic T cell (CTL) activity. Binding of F protein-derived peptides to HLA-A*0201 was studied to identify putative CTL epitopes.

As such, it was determined that sera from 23 of 39 patients infected with various HCV genotypes recognized the truncated form, including 13 of 25 subjects coinfected with HIV-1, indicative of antigenic cross-reactivity and consistent with conservation of F protein coding sequences between HCV genotypes. F protein-specific CTL precursors were detected in 9 of 11 HCV-infected subjects, including 7 of 9 patients coinfected with HCV and HIV-1. Finally, 3 novel putative HLA-A*0201-restricted CTL epitopes were identified.

The results described herein show that cross-reactive F protein-specific immunoglobulin and CTL responses can be equally detected in HCV infected hosts and in subjects co-infected with HIV-1.

Accordingly, in a first aspect, the invention relates to a polypeptide derived from an HCV F protein, with the proviso that the polypeptide is not the full length HCV F protein set forth in SEQ ID NO: 8. Such a polypeptide includes the HCV Fmut8 protein, truncated versions thereof (e.g. Δ11 version, lacking 11 amino acids from the N-terminus), as well as truncated versions of the HCV F protein set forth in SEQ ID NO: 8. The invention further relates to a polypeptide epitope of the HCV F protein or HCV Fmut8 protein, i.e. an immunogenic polypeptide or fragment derived from these proteins, as well as to variants or fragments of the polypeptide. In embodiments, the polypeptide or variants or fragments thereof have or are capable of effecting or eliciting an activity including but not limited to an induction of T-cell lytic activity, binding to an HLA or MHC class I molecule, immunoreactivity with serum from an HCV-infected subject, an alteration in cytokine or chemokine expression or production, or any combination thereof.

In embodiments, the polypeptide may be less than 50, 45, 40, 35, 30, 25, 20, 15 or 10 amino acids in length. In embodiments the polypeptide is greater than or equal to 5, 8 or 9 amino acids in length. In embodiments the polypeptide is 9, 10 or 15 amino acids in length.

In embodiments, the polypeptide comprises an amino acid sequence selected from SEQ ID NOs: 9, 11, 12 and/or 14 to 38, 51 to 103 and/or 122 to 128; or a functional (e.g. immunogenic) variant or fragment thereof. In embodiments, the polypeptide comprises 1-5, 1-10, 1-15, 1-20, 1-30 or 1-40 amino acid additions to an amino acid sequence selected from 9, 11, 12, 14 to 38 and/or 51 to 103. In a further embodiment, the polypeptide consists essentially of an amino acid sequence selected from 9, 11, 12, 14 to 38 and/or 51 to 103.

In embodiments, the polypeptide consists essentially of 5-10, 9, 10, 5-15, 15, 5-20, 5-25, 5-30, 30, 5-35, 5-40, 5-45, 45 or 5-50 contiguous amino acids of an amino acid sequence selected from SEQ ID NOs: 9, 11, 12 and/or 122 to 128.

In embodiments, the polypeptide or epitope of the invention comprises an amino acid sequence having the following general formula I (see Table 14):

X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³

wherein;
X¹ is selected from V, A and D or is absent;
X² is R or is absent;
X³ is S or is absent;
X⁴ is L or is absent;
X⁵ is selected from V and A or is absent;
X⁶ is E or is absent;
X⁷ is selected from F and Y or is absent;
X⁸ is T or is absent;

### X⁹ is C;

### X¹⁰ is C;

### X¹¹ is R;

### X¹² is A;

X¹³ is selected from G and R or is absent;
X¹⁴ is A or is absent;
X¹⁵ is selected from L, P and H or is absent;
X¹⁶ is selected from D, G and N or is absent;
X¹⁷ is W or is absent;
X¹⁸ is V or is absent;
X¹⁹ is selected from C and S or is absent;
X²⁰ is A or is absent;
X²¹ is R or is absent;
X²² is selected from R, Q and L or is absent; and
X²³ is selected from E, G and V or is absent.

In further embodiments, the polypeptide or epitope of the invention comprises an amino acid sequence having the following general formula II (see Table 15):

Z¹-Z²-Z³-Z⁴-Z⁵-Z⁶-Z⁷-Z⁸-Z⁹-Z¹⁰-Z¹¹-Z¹²-Z¹³-Z¹⁴-Z¹⁵

wherein;
Z¹ is selected from P and H or is absent;
Z² is selected from V, E and E or is absent;
Z³ is selected from V and A or is absent;
Z⁴ is selected from L and P or is absent;
Z⁵ is selected from G, V and D or is absent;
Z⁶ is selected from L, P, H and R or is absent;
Z⁷ is selected from A, L, P, I, T and V or is absent;

### Z⁸ is G;

### Z⁹ is A;

Z¹⁰ is selected from P and Q;
Z¹¹ is selected from Q and M;

### Z¹² is T;

### Z¹³ is P;

Z¹⁴ is selected from G and A;
Z¹⁵ is selected from V, I and G.

The invention further provides a pharmaceutical composition, such as a vaccine or immunogenic composition, comprising the polypeptide and a pharmaceutically acceptable carrier. In a further embodiment, the composition further comprises an adjuvant. In a further embodiment, the composition further comprises an MHC molecule.

The invention further provides a multimer (i.e. 2 or more) of MHC peptide complexes, whereby each MHC complex comprises a polypeptide of the invention and an MHC molecule. In an embodiment, the MHC complex comprises a polypeptide of the invention, an MHC class I heavy chain and β2 microglobulin. Such multimer systems are known in the art, and the complexes may for example be associated together via suitable interactions with a multivalent entity, e.g. biotin-(strept) avidin (which are tetravalent thus resulting in a tetramer) interactions (see U.S. Pat. No. 5,635,363 [Jun. 3, 1997}; Altman et al., Science, 1996 Oct. 4; 274(5284): 94-6. Erratum in: Science 1998 Jun. 19; 280(5371):1821.). Such multimers can also be used to label, detect, isolate, and stimulate T cells, as well as to discover other epitopes.

In an embodiment, a composition (e.g. a vaccine or immunogenic composition) of the invention may comprise a plurality of the polypeptides of the invention. In an embodiment the composition may comprise a second polypeptide capable of eliciting a HCV immune response, such as an additional HCV polypeptide or fragment thereof.

The invention further provides an antibody against, or which recognizes, or is capable of specifically binding to a polypeptide of the invention.

The invention further provides an isolated nucleic acid or polynucleotide which encodes a polypeptide of the invention (such as a nucleotide sequence selected from SEQ ID NO: 10 or a fragment thereof, or a sequence which differs therefrom but still encodes the same polypeptide by virtue of the degeneracy of the genetic code).

The invention further provides a vector comprising the nucleic acid operably-linked to a transcriptional regulatory or expression control sequence (e.g. a promoter). The invention further provides a host cell comprising the nucleic acid or vector.

The invention further provides prophylactic and therapeutic methods, for preventing or treating HCV infection, comprising administering a polypeptide, composition, or MHC complex (comprising a polypeptide of the invention and one or more MHC molecules [e.g. MHC class I heavy chain, β2 microglobulin]) or vector of the invention to an animal (e.g., a mammal, e.g., a human). In embodiments, such methods comprise administering a polypeptide, composition or vector of the invention to vaccinate or immunize (i.e. generate an immune response) in an animal. In an embodiment, the animal also suffers from HIV infection.

The invention further provides diagnostic methods for the diagnosis and detection of HCV infection. Such methods may utilize as a reagent a polypeptide or composition of the invention, a multimer comprising 2 or more MHC peptide complexes as noted above, or an antibody which binds specifically to a polypeptide of the invention. In embodiments, the method comprises contacting a biological sample, such as a tissue or body fluid (e.g. blood, lymphocytes) of an animal, with the reagent. The invention further provides a peptide array or microarray comprising a polypeptide of the invention, and optionally other components such as an MHC molecule, which may be used in the just-noted diagnostic methods.

The invention further provides a method of detection or quantitation of HCV in a biological sample. Such methods may utilize as a reagent a polypeptide or composition of the invention, a multimer comprising 2 or more MHC peptide complexes as noted above, or an antibody which binds specifically to a polypeptide of the invention. In embodiments, the method comprises contacting the biological sample, such as a tissue or body fluid (e.g. blood, lymphocytes) of an animal, with the reagent. The invention further provides a peptide array or microarray comprising a polypeptide of the invention, and optionally other components such as an MHC molecule, which may be used in the just-noted method.

The invention further provides a use of the polypeptide, MHC complex (comprising a polypeptide of the invention and one or more MHC molecules [e.g. MHC class I heavy chain, β2 microglobulin]) or vector of the invention for the preparation of a medicament or vaccine, e.g. for the prevention or treatment of HCV infection. The invention further provides a use of the polypeptide, composition or vector of the invention for the prevention or treatment of HCV infection.

The invention further provides a commercial package comprising a polypeptide, composition or vector of the invention together with instructions for the prevention or treatment of HCV infection.

The invention further provides a commercial package comprising a polypeptide, composition or antibody of the invention together with instructions for the diagnosis and detection of HCV infection.

The invention further relates to a fusion polypeptide. As used herein, a fusion polypeptide is one that contains a polypeptide or a polypeptide derivative of the invention fused at the N- or C-terminal end to any other polypeptide (hereinafter referred to as a peptide tail). A simple way to obtain such a fusion polypeptide is by translation of an in-frame fusion of the polynucleotide sequences, i.e., a hybrid sequence. The hybrid sequence encoding the fusion polypeptide is inserted into an expression vector which is used to transform or transfect a host cell. Alternatively, the polynucleotide sequence encoding the polypeptide or polypeptide derivative is inserted into an expression vector in which the polynucleotide encoding the peptide tail is already present. Such vectors and instructions for their use are commercially available, e.g. the pMal-c2 or pMal-p2 system from New England Biolabs, in which the peptide tail is a maltose binding protein, the glutathione-S-transferase system of Pharmacia, or the His-Tag system available from Novagen. These and other expression systems provide convenient means for further purification of polypeptides and derivatives of the invention. An advantageous example of a fusion polypeptide is one where the polypeptide or homolog or fragment of the invention is fused to a polypeptide having adjuvant activity, such as subunit B of either cholera toxin or E. coli heat-labile toxin.

To effect fusion, the polypeptide of the invention is fused to the N—, or preferably, to the C-terminal end of the polypeptide having adjuvant activity. Alternatively, a polypeptide fragment of the invention is inserted internally within the amino acid sequence of the polypeptide having adjuvant activity.

Consistent with the above, the polynucleotides of the invention also encode hybrid precursor polypeptides containing heterologous signal peptides, which mature into polypeptides of the invention. By “heterologous signal peptide” is meant a signal peptide that is not found in naturally-occurring precursors of polypeptides of the invention.

Polynucleotide molecules according to the invention, including RNA, DNA, or modifications or combinations thereof, have various applications. A DNA molecule is used, for example, (i) in a process for producing the encoded polypeptide in a recombinant host system, (ii) in the construction of vaccine vectors such as poxviruses, which are further used in methods and compositions for preventing and/or treating HCV infection, and (iii) as a vaccine agent (as well as an RNA molecule), in a naked form or formulated with a delivery vehicle.

Accordingly, a further aspect of the invention encompasses (i) an expression cassette containing a DNA molecule of the invention placed under the control of the elements required for expression, in particular under the control of an appropriate promoter; (ii) an expression vector containing an expression cassette of the invention; (iii) a prokaryotic or eukaryotic cell transformed or transfected with an expression cassette and/or vector of the invention, as well as (iv) a process for producing a polypeptide or polypeptide derivative encoded by a polynucleotide of the invention, which involves culturing a procaryotic or eucaryotic cell transformed or transfected with an expression cassette and/or vector of the invention, under conditions that allow expression of the DNA molecule of the invention and, recovering the encoded polypeptide or polypeptide derivative from the cell culture.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

In another aspect of the invention, an isolated nucleic acid, for example a nucleic acid sequence encoding a polypeptide of the invention, or homolog, fragment or variant thereof, may further be incorporated into a recombinant expression vector. In an embodiment, the vector will comprise transcriptional regulatory sequences or a promoter operably-linked to a nucleic acid comprising a sequence capable of encoding a peptide compound, polypeptide or domain of the invention. A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked.

A recombinant expression system is selected from prokaryotic and eukaryotic hosts. Eukaryotic hosts include yeast cells (e.g., Saccharomyces cerevisiae or Pichia pastoris), mammalian cells (e.g., COS1, NIH3T3, or JEG3 cells), arthropods cells (e.g., Spodoptera frugiperda (SF9) cells), and plant cells. A preferred expression system is a prokaryotic host such as E. coli. Bacterial and eukaryotic cells are available from a number of different sources including commercial sources to those skilled in the art, e.g., the American Type Culture Collection (ATCC; Rockville, Md.). Commercial sources of cells used for recombinant protein expression also provide instructions for usage of the cells.

The choice of the expression system depends on the features desired for the expressed polypeptide. For example, it may be useful to produce a polypeptide of the invention in a particular lipidated form or any other form.

One skilled in the art would readily understand that not all vectors and expression control sequences and hosts would be expected to express equally well the polynucleotides of this invention. With the guidelines described below, however, a selection of vectors, expression control sequences and hosts may be made without undue experimentation and without departing from the scope of this invention.

In selecting a vector, a host is chosen that is compatible with the vector which is to exist and possibly replicate in it. Considerations are made with respect to the vector copy number, the ability to control the copy number, expression of other proteins such as antibiotic resistance. In selecting an expression control sequence, a number of variables are considered. Among the important variables are the relative strength of the sequence (e.g. the ability to drive expression under various conditions), the ability to control the sequence's function, compatibility between the polynucleotide to be expressed and the control sequence (e.g. secondary structures are considered to avoid hairpin structures which prevent efficient transcription). In selecting the host, unicellular hosts are selected which are compatible with the selected vector, tolerant of any possible toxic effects of the expressed product, able to secrete the expressed product efficiently if such is desired, able to express the product in the desired conformation, able to be easily scaled up, and from which the final product can be easily purified.

The choice of the expression cassette depends on the host system selected as well as the features desired for the expressed polypeptide. Typically, an expression cassette includes a promoter that is functional in the selected host system and can be constitutive or inducible; a ribosome binding site; a start codon (ATG) if necessary; a region encoding a signal peptide, e.g., a lipidation signal peptide; a DNA molecule of the invention; a stop codon; and optionally a 3′ terminal region (translation and/or transcription terminator). The signal peptide encoding region is adjacent to the polynucleotide of the invention and placed in proper reading frame. The signal peptide-encoding region is homologous or heterologous to the DNA molecule encoding the mature polypeptide and is compatible with the secretion apparatus of the host used for expression. The open reading frame constituted by the DNA molecule of the invention, solely or together with the signal peptide, is placed under the control of the promoter so that transcription and translation occur in the host system. Promoters and signal peptide encoding regions are widely known and available to those skilled in the art and include, for example, the promoter of Salmonella typhimurium (and derivatives) that is inducible by arabinose (promoter araB) and is functional in Gram-negative bacteria such as E. coli (as described in U.S. Pat. No. 5,028,530 and in Cagnon et al., (Cagnon et al., Protein Engineering (1991) 4(7):843)); the promoter of the gene of bacteriophage T7 encoding RNA polymerase, that is functional in a number of E. coli strains expressing T7 polymerase (described in U.S. Pat. No. 4,952,496); OspA lipidation signal peptide; and RlpB lipidation signal peptide (Takase et al., J. Bact. (1987) 169:5692).

The expression cassette is typically part of an expression vector, which is selected for its ability to replicate in the chosen expression system. Expression vectors (e.g., plasmids or viral vectors) can be chosen, for example, from those described in Pouwels et al. (Cloning Vectors: A Laboratory Manual 1985, Supp. 1987). Suitable expression vectors can be purchased from various commercial sources.

Methods for transforming/transfecting host cells with expression vectors are well-known in the art and depend on the host system selected as described in Ausubel et al. (Ausubel et al., Current Protocols in Molecular Biology, John Wiley & Sons Inc., 1994).

Upon expression, a recombinant polypeptide of the invention (or a polypeptide derivative) is produced and remains in the intracellular compartment, is secreted/excreted in the extracellular medium or in the periplasmic space, or is embedded in the cellular membrane. The polypeptide is recovered in a substantially purified form from the cell extract or from the supernatant after centrifugation of the recombinant cell culture. Typically, the recombinant polypeptide is purified by antibody-based affinity purification or by other well-known methods that can be readily adapted by a person skilled in the art, such as fusion of the polynucleotide encoding the polypeptide or its derivative to a small affinity binding domain. Antibodies useful for purifying by immunoaffinity the polypeptides of the invention are obtained as described below.

In embodiments, the invention further provides nucleic acid and polypeptide variants which are homologous or substantially identical to a nucleic acid or polypeptide of the invention (e.g., any of SEQ ID Nos: 1-128). Such variants may differ from a nucleic acid or polypeptide of the invention by substitution, deletion and/or addition of one or more residues (nucleotide or amino acid, as appropriate).

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with any of SEQ ID Nos: 1-128.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95%, of any of SEQ ID NOs: 1-128. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridisation to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology —Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

In an aspect, a polypeptide of the invention is substantially purified. A “substantially purified polypeptide” as used herein is defined as a polypeptide that is separated from the environment in which it naturally occurs and/or that is free of the majority of the polypeptides that are present in the environment in which it was synthesized. For example, a substantially purified polypeptide is free from cytoplasmic polypeptides. Those skilled in the art would readily understand that the polypeptides of the invention may be chemically synthesized, produced by recombinant means, or generated from a natural source.

In an embodiment, a polypeptide of the invention is a recombinant polypeptide.

In an aspect, the invention provides a method for producing a polypeptide that results in no or substantially no production of an HCV protein other than the HCV F protein (e.g. an HCV core protein). The invention further provides a polypeptide of the invention or a preparation comprising said polypeptide which is substantially free of an HCV protein other than the HCV F protein (e.g. an HCV core protein).

As used herein, the immunogenic or vaccine compositions of the invention are administered by conventional routes known the vaccine field, in such as to a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g. via a patch). The choice of administration route depends upon a number of parameters, such as the adjuvant associated with the polypeptide. If a mucosal adjuvant is used, the intranasal or oral route is preferred. If a lipid formulation or an aluminum compound is used, the parenteral route is preferred with the sub-cutaneous or intramuscular route being most preferred. The choice also depends upon the nature of the vaccine agent.

For use in a composition of the invention, a polypeptide or derivative thereof is formulated into or with liposomes, preferably neutral or anionic liposomes, microspheres, ISCOMS, or virus-like-particles (VLPs) to facilitate delivery and/or enhance the immune response. These compounds are readily available to one skilled in the art; for example, see Liposomes: A Practical Approach, RCP New Ed, IRL press (1990).

Adjuvants other than liposomes and the like are also used and are known in the art. Adjuvants may protect the antigen from rapid dispersal by sequestering it in a local deposit, or they may contain substances that stimulate the host to secrete factors that are chemotactic for macrophages and other components of the immune system. An appropriate selection can conventionally be made by those skilled in the art, for example, from those described below.

A polynucleotide of the invention can also be useful as a vaccine. There are two major routes, either using a viral or bacterial host as gene delivery vehicle (live vaccine vector) or administering the gene in a free form, e.g., inserted into a plasmid. Therapeutic or prophylactic efficacy of a polynucleotide of the invention is evaluated as described below.

Accordingly, a further aspect of the invention provides (i) a vaccine vector such as a poxvirus, containing a DNA molecule of the invention, placed under the control of elements required for expression; (ii) a composition of matter comprising a vaccine vector of the invention, together with a diluent or carrier; specifically (iii) a pharmaceutical composition containing a therapeutically or prophylactically effective amount of a vaccine vector of the invention; (iv) a method for inducing an immune response against HCV in a mammal (e.g., a human; alternatively, the method can be used in veterinary applications for treating or preventing HCV infection of non-human animals), which involves administering to the mammal an immunogenically effective amount of a vaccine vector of the invention to elicit a protective or therapeutic immune response to HCV; and particularly, (v) a method for preventing and/or treating HCV infection, which involves administering a prophylactic or therapeutic amount of a vaccine vector of the invention to an infected individual. Additionally, the invention further provides a use of a vaccine vector of the invention in the preparation of a medicament for preventing and/or treating HCV infection.

As used herein, a vaccine vector expresses one or several polypeptides or derivatives of the invention. The vaccine vector may express additionally a cytokine, such as interleukin-2 (IL-2) or interleukin-12 (IL-12), that enhances the immune response (adjuvant effect). It is understood that each of the components to be expressed is placed under the control of elements required for expression in a mammalian cell.

The invention further provides a composition comprising several polypeptides or derivative thereof of the invention or vaccine vectors (each of them capable of expressing a polypeptide or derivative thereof of the invention). A composition may also comprise an additional HCV antigen, or a subunit, fragment, homolog, mutant, or derivative thereof; optionally together with or a cytokine such as IL-2 or IL-12 (or vaccine vector(s) capable of their expression).

“Vaccine” as used herein refers to a composition or formulation comprising one or more polypeptides/peptides of the invention, or a vaccine vector of the invention. Vaccination methods for treating or preventing infection in a mammal comprises use of a vaccine or vaccine vector of the invention to be administered by any conventional route.

Treatment may be effected in a single dose or repeated at intervals. The appropriate dosage depends on various parameters understood by skilled artisans such as the vaccine or vaccine vector itself, the route of administration or the condition of the mammal to be vaccinated (weight, age and the like).

Live vaccine vectors available in the art include viral vectors such as adenoviruses and poxviruses as well as bacterial vectors, e.g., Shigella, Salmonella, Vibrio cholerae, Lactobacillus, Bacille Calmette-Guérin (BCG), and Streptococcus.

An example of an adenovirus vector, as well as a method for constructing an adenovirus vector capable of expressing a DNA molecule of the invention, are described in U.S. Pat. No. 4,920,209. Poxvirus vectors include vaccinia and canary pox virus, described in U.S. Pat. No. 4,722,848 and U.S. Pat. No. 5,364,773, respectively. (Also see, e.g., Tartaglia et al., Virology (1992) 188:217) for a description of a vaccinia virus vector and Taylor et al, Vaccine (1995) 13:539 for a description of a canary pox vector) Poxvirus vectors capable of expressing a polynucleotide of the invention are obtained by homologous recombination as described in Kieny et al., Nature (1984) 312:163 so that the polynucleotide of the invention is inserted in the viral genome under appropriate conditions for expression in mammalian cells. Generally, the dose of vaccine viral vector, for therapeutic or prophylactic use, can be of from about 1×10⁴ to about 1×10¹¹, advantageously from about 1×10⁷ to about 1×10¹⁰, preferably of from about 1×10⁷ to about 1×10⁹ plaque-forming units per kilogram. Preferably, viral vectors are administered parenterally; for example, in 3 doses, 4 weeks apart. It is preferable to avoid adding a chemical adjuvant to a composition containing a viral vector of the invention and thereby minimizing the immune response to the viral vector itself.

Non-toxicogenic Vibrio cholerae mutant strains that are useful as a live oral vaccine are known. Mekalanos et al., Nature (1983) 306:551 and U.S. Pat. No. 4,882,278 describe strains which have a substantial amount of the coding sequence of each of the two ctxA alleles deleted so that no functional cholerae toxin is produced. WO 92/11354 describes a strain in which the irgA locus is inactivated by mutation; this mutation can be combined in a single strain with ctxA mutations. WO 94/01533 describes a deletion mutant lacking functional ctxA and attRS1 DNA sequences. These mutant strains are genetically engineered to express heterologous antigens, as described in WO 94/19482. An effective vaccine dose of a Vibrio cholerae strain capable of expressing a polypeptide or polypeptide derivative encoded by a DNA molecule of the invention contains about 1×10⁵ to about 1×10⁹, preferably about 1×10⁶ to about 1×10⁸, viable bacteria in a volume appropriate for the selected route of administration. Preferred routes of administration include all mucosal routes; most preferably, these vectors are administered intranasally or orally.

Attenuated Salmonella typhimurium strains, genetically engineered for recombinant expression of heterologous antigens or not, and their use as oral vaccines are described in Nakayama et al. (Bio/Technology (1988) 6:693) and WO 92/11361. Preferred routes of administration include all mucosal routes; most preferably, these vectors are administered intranasally or orally.

Other bacterial strains used as vaccine vectors in the context of the present invention are described for Shigella flexneri in High et al., EMBO (1992) 11:1991 and Sizemore et al., Science (1995) 270:299; for Streptococcus gordonii in Medaglini et al., Proc. Natl. Acad. Sci. USA (1995) 92:6868; and for Bacille Calmette Guerin in Flynn J. L., Cell. Mol. Biol. (1994) 40 (suppl. I):31, WO 88/06626, WO 90/00594, WO 91/13157, WO 92/01796, and WO 92/21376.

In bacterial vectors, the polynucleotide of the invention is inserted into the bacterial genome or remains in a free state as part of a plasmid.

The composition comprising a polypeptide or vaccine vector of the present invention may further contain an adjuvant. A number of adjuvants are known to those skilled in the art. Preferred adjuvants are selected as described below.

Accordingly, a further aspect of the invention provides (i) a composition of matter comprising a polypeptide or polynucleotide of the invention, together with a diluent or carrier; (ii) a pharmaceutical composition comprising a therapeutically or prophylactically effective amount of a polypeptide or polynucleotide of the invention; (iii) a method for inducing an immune response against HCV in a mammal by administration of an immunogenically effective amount of a polypeptide or polynucleotide of the invention to elicit a protective immune response to HCV; and particularly, (iv) a method for preventing and/or treating a HCV infection, by administering a prophylactic or therapeutic amount of a polypeptide or polynucleotide of the invention to an infected individual. Additionally, the invention further provides a use of a polypeptide or polynucleotide of the invention in the preparation of a medicament for preventing and/or treating HCV infection.

Use of the polynucleotides of the invention include their administration to a mammal as a vaccine, for therapeutic or prophylactic purposes. Such polynucleotides are used in the form of DNA as part of a plasmid that is unable to replicate in a mammalian cell and unable to integrate into the mammalian genome. Typically, such a DNA molecule is placed under the control of a promoter suitable for expression in a mammalian cell. The promoter functions either ubiquitously or tissue-specifically. Examples of non-tissue specific promoters include the early Cytomegalovirus (CMV) promoter (described in U.S. Pat. No. 4,168,062) and the Rous Sarcoma Virus promoter (described in Norton & Coffin, Molec. Cell Biol. (1985) 5:281). An example of a tissue-specific promoter is the desmin promoter which drives expression in muscle cells (Li et al., Gene (1989) 78:243, Li & Paulin, J. Biol. Chem. (1991) 266:6562 and Li & Paulin, J. Biol. Chem. (1993) 268:10403). Use of promoters is well-known to those skilled in the art. Useful vectors are described in numerous publications, specifically WO 94/21797 and Hartikka et al., Human Gene Therapy (1996) 7:1205.

Polynucleotides of the invention which are used as vaccines encode either a precursor or a mature form of the corresponding polypeptide. In the precursor form, the signal peptide is either homologous or heterologous. In the latter case, a eukaryotic leader sequence such as the leader sequence of the tissue-type plasminogen factor (tPA) is preferred.

Standard techniques of molecular biology for preparing and purifying polynucleotides are used in the preparation of polynucleotide therapeutics of the invention. For use as a vaccine, a polynucleotide of the invention is formulated according to various methods outlined below.

One method utilizes the polynucleotide in a naked form, free of any delivery vehicles. Such a polynucleotide is simply diluted in a physiologically acceptable solution such as sterile saline or sterile buffered saline, with or without a carrier. When present, the carrier preferably is isotonic, hypotonic, or weakly hypertonic, and has a relatively low ionic strength, such as provided by a sucrose solution, e.g., a solution containing 20% sucrose.

An alternative method utilizes the polynucleotide in association with agents that assist in cellular uptake. Examples of such agents are (i) chemicals that modify cellular permeability, such as bupivacaine (see, e.g., WO 94/16737), (ii) liposomes for encapsulation of the polynucleotide, or (iii) cationic lipids or silica, gold, or tungsten microparticles which associate themselves with the polynucleotides.

Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides.

Cationic lipids are also known in the art and are commonly used for gene delivery. Such lipids include Lipofectin™ also known as DOTMA (N-[1-(2,3-dioleyloxy)propyl]-N,N,N-trimethylammonium chloride), DOTAP (1,2-bis(oleyloxy)-3-(trimethylammonio)propane), DDAB (dimethyldioctadecylammonium bromide), DOGS (dioctadecylamidologlycyl spermine) and cholesterol derivatives such as DC-Chol (3 beta-(N—(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. Cationic lipids for gene delivery are preferably used in association with a neutral lipid such as DOPE (dioleyl phosphatidylethanolamine), as described in WO 90/11092 as an example.

Formulation containing cationic liposomes may optionally contain other transfection-facilitating compounds. A number of them are described in WO 93/18759, WO 93/19768, WO 94/25608, and WO 95/02397. They include spermine derivatives useful for facilitating the transport of DNA through the nuclear membrane (see, for example, WO 93/18759) and membrane-permeabilizing compounds such as GALA, Gramicidine S, and cationic bile salts (see, for example, WO 93/19768).

Gold or tungsten microparticles are used for gene delivery, as described in WO 91/00359, WO 93/17706, and Tang et al. Nature (1992) 356:152. The microparticle-coated polynucleotide is injected via intradermal or intraepidermal routes using a needleless injection device (“gene gun”), such as those described in U.S. Pat. No. 4,945,050, U.S. Pat. No. 5,015,580, and WO 94/24263.

The amount of DNA to be used in a vaccine recipient depends, e.g., on the strength of the promoter used in the DNA construct, the immunogenicity of the expressed gene product, the condition of the mammal intended for administration (e.g., the weight, age, and general health of the mammal), the mode of administration, and the type of formulation. In general, a therapeutically or prophylactically effective dose from about 1 μg to about 1 mg, preferably, from about 10 μg to about 800 μg and, more preferably, from about 25 μg to about 250 μg, can be administered to human adults. The administration can be achieved in a single dose or repeated at intervals.

Although not absolutely required, such a composition can also contain an adjuvant. If so, a systemic adjuvant that does not require concomitant administration in order to exhibit an adjuvant effect is preferable such as, e.g., QS21, which is described in U.S. Pat. No. 5,057,546.

Treatment is achieved in a single dose or repeated as necessary at intervals, as can be determined readily by one skilled in the art. For example, a priming dose is followed by three booster doses at weekly or monthly intervals. An appropriate dose depends on various parameters including the recipient (e.g., adult or infant), the particular vaccine antigen, the route and frequency of administration, the presence/absence or type of adjuvant, and the desired effect (e.g., protection and/or treatment), as can be determined by one skilled in the art. In an embodiment, a polypeptide of the invention, administered as a vaccine, is administered by a mucosal route in an amount from about 10 μg to about 500 mg, preferably from about 1 mg to about 200 mg. For the parenteral route of administration, the dose usually does not exceed about 1 mg, preferably about 100 μg.

When used as vaccine agents, polypeptides and polynucleotides of the invention may be used sequentially as part of a multistep immunization process. For example, a mammal is initially primed with a vaccine vector of the invention such as a pox virus, e.g., via the parenteral route, and then boosted twice with the polypeptide encoded by the vaccine vector, e.g., via the mucosal route. In another example, liposomes associated with a polypeptide or derivative of the invention are also used for priming, with boosting being carried out mucosally using a soluble polypeptide or derivative of the invention in combination with a mucosal adjuvant (e.g., LT).

A polypeptide or variant or derivative thereof of the invention is also used in accordance with a further aspect of the invention as a diagnostic reagent for detecting the presence of anti-HCV antibodies, e.g., in a blood sample. Such polypeptides are about 5 to about 80, preferably about 10 to about 50 amino acids in length. They are either labeled or unlabeled, depending upon the diagnostic method. Diagnostic methods involving such a reagent are described below.

Adjuvants useful in any of the vaccine compositions described above are as follows.

Adjuvants for parenteral administration include aluminum compounds, such as aluminum hydroxide, aluminum phosphate, and aluminum hydroxy phosphate. The antigen is precipitated with, or adsorbed onto, the aluminum compound according to standard protocols. Other adjuvants, such as RIBI (ImmunoChem, Hamilton, Mont.), are used in parenteral administration.

Adjuvants for mucosal administration include bacterial toxins, e.g., the cholera toxin (CT), the E. coli heat-labile toxin (LT), the Clostridium difficile toxin A and the pertussis toxin (PT), or combinations, subunits, toxoids, or mutants thereof such as a purified preparation of native cholera toxin subunit B (CTB). Fragments, homologs, derivatives, and fusions to any of these toxins are also suitable, provided that they retain adjuvant activity. Preferably, a mutant having reduced toxicity is used. Suitable mutants are described, e.g., in WO 95/17211 (Arg-7-Lys CT mutant), WO 96/06627 (Arg-192-Gly LT mutant), and WO 95/34323 (Arg-9-Lys and Glu-129-Gly PT mutant). Additional LT mutants that are used in the methods and compositions of the invention include, e.g., Ser-63-Lys, Ala-69Gly, Glu-110-Asp, and Glu-112-Asp mutants. Other adjuvants, such as a bacterial monophosphoryl lipid A (MPLA) of, e.g., E. coli, Salmonella minnesota, Salmonella typhimurium, or Shigella flexneri; saponins, or polylactide glycolide (PLGA) microspheres, is also be used in mucosal administration.

Adjuvants useful for both mucosal and parenteral administrations include polyphosphazene (WO 95/02415), DC-chol (3 b-(N—(N′,N′-dimethyl aminomethane)-carbamoyl) cholesterol; U.S. Pat. No. 5,283,185 and WO 96/14831) and QS-21 (WO 88/09336).

Any pharmaceutical composition of the invention containing a polypeptide, a polypeptide derivative, a polynucleotide or an antibody of the invention, is manufactured in a conventional manner. In particular, it is formulated with a pharmaceutically acceptable diluent or carrier, e.g., water or a saline solution such as phosphate buffer saline. In general, a diluent or carrier is selected on the basis of the mode and route of administration, and standard pharmaceutical practice. Suitable pharmaceutical carriers or diluents, as well as pharmaceutical necessities for their use in pharmaceutical formulations, are described in Remington's Pharmaceutical Sciences, a standard reference text in this field and in the USP/NF.

A “therapeutically effective amount” refers to an amount effective, at dosages and for periods of time necessary, to achieve the desired therapeutic result, such as a reduction of symptoms of HCV infection and in turn a reduction in progression of HCV infection and associated disease and an improvement in prognosis of HCV infection and associated disease. A therapeutically effective amount may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting onset or progression of HCV infection and associated symptoms and disease. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, a polypeptide of the invention can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

A further aspect of the invention provides an antibody that recognizes the polypeptide of the invention.

An antibody of the invention is either polyclonal or monoclonal. Antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monoclonal antibodies may also be in the form of immunoglobulin fragments, e.g., F(ab)′₂, Fab or Fab′ fragments. The antibodies of the invention are of any isotype, e.g., IgG or IgA, and polyclonal antibodies are of a single isotype or a mixture of isotypes.

Antibodies against the polypeptide of the present invention are generated by immunization of a mammal with a partially purified fraction comprising the polypeptide. Such antibodies may be polyclonal or monoclonal. Methods to produce polyclonal or monoclonal antibodies are well known in the art. For a review, see Harlow and Lane (1988) and Yelton et al. (1981), both of which are herein incorporated by reference. For monoclonal antibodies, see Kohler and Milstein (1975), herein incorporated by reference.

The antibodies of the invention, which are raised to a partially purified fraction comprising the polypeptide of the invention, are produced and identified using standard immunological assays, e.g., Western blot analysis, dot blot assay, or ELISA (see, e.g., Coligan et al. (1994), herein incorporated by reference). The antibodies are used in diagnostic methods to detect the presence of a HCV F protein or fragment thereof or HCV in a sample, such as a tissue or body fluid. The antibodies are also used in affinity chromatography for obtaining a purified fraction comprising the polypeptide of the invention.

Accordingly, a further aspect of the invention provides (i) a reagent for detecting the presence of a HCV F protein polypeptide or fragment thereof and/or HCV in a tissue or body fluid; and (ii) a diagnostic method for detecting the presence of a HCV F protein polypeptide or fragment thereof and/or HCV in a tissue or body fluid, by contacting the tissue or body fluid with an antibody of the invention, such that an immune complex is formed, and by detecting such complex to indicate the presence of a HCV F protein polypeptide or fragment thereof and/or HCV in the sample or the organism from which the sample is derived.

Those skilled in the art will readily understand that the immune complex is formed between a component of the sample and the antibody, and that any unbound material is removed prior to detecting the complex. It is understood that an antibody of the invention is used for screening a sample, such as, for example, blood, plasma, lymphocytes, cerebrospinal fluid, urine, saliva, epithelia and fibroblasts, for the presence of a HCV F protein polypeptide or fragment thereof and/or HCV.

Similarly, a polypeptide of the invention may be used as a reagent to detect the presence of an antibody to a HCV F protein or fragment thereof and/or HCV in a tissue or body fluid, and therefore the invention further provides such a reagent, as well as a diagnostic method for detecting the presence of a an antibody to a HCV F protein or fragment thereof and/or HCV, by contacting the tissue or body fluid with a polypeptide of the invention, such that an immune complex is formed, and by detecting such complex to indicate the presence of a HCV F protein or fragment thereof and/or HCV in the sample or the organism from which the sample is derived.

For diagnostic applications, the reagent (e.g, the polypeptide or antibody of the invention) is either in a free state or immobilized on a solid support, such as a tube, a bead, a plate or well thereof, or any other conventional support used in the field (such as a peptide microarray). Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-streptavidin system. Alternatively, a peptide tail is added chemically or by genetic engineering to the reagent and the grafted or fused product immobilized by passive adsorption or covalent linkage of the peptide tail.

Such diagnostic agents may be included in a kit which also comprises instructions for use. The reagent is labeled with a detection means which allows for the detection of the reagent when it is bound to its target. The detection means may be a fluorescent agent such as fluorescein isocyanate or fluorescein isothiocyanate, or an enzyme such as horseradish peroxidase or luciferase or alkaline phosphatase, or a radioactive element such as ¹²⁵I or ⁵¹Cr.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Example 1

### Materials and Methods

### Study Subjects and Clinical Parameters.

For studies of humoral immune responses, 39 female patients were selected among the participants of the Centre maternel et infantile sur le SIDA (CMIS) mother-child-cohort (CHU mere-enfant Sainte-Justine, Montreal, Canada). Mean age was 29.6+/−5.75 years (range-20.1-41.3 years). HCV infection was confirmed by ELISA and recombinant immunoblot assays, in accordance with clinical practice and diagnostic algorithms used in the Province of Quebec. Mean HCV RNA level (COBAS Amplicor HCV Monitor assay version 2.0, Roche Diagnostics, Montreal, Canada) was 5.37 log IU/ml plasma (range=2.78-7.09 log IU/ml plasma). HCV genotyping was performed by sequence analysis of the 5′ non-coding region, as described [18]. Co-infection with HIV-1 was confirmed by ELISA and non-quantitative PCR in 25 of these 39 subjects. In coinfected patients, mean CD4 cell count, measured using flow cytometry, was 509+/−243 cells/mm³ (range=33-1287 cells/mm³), and mean HIV-1 viral load (Versant HIV RNA version 3.0 assay, Bayer, Pittsburgh, Pa.) was 2.81 log RNA copies/ml plasma (range=1.70-4.73 log RNA copies/ml plasma). Nineteen of these 25 subjects were treated with antiretroviral therapy, including single agent (n=3), double combination therapy (n=6), and triple combination therapy (n=11). Reported HIV risk categories included injection drug use (n=22), probable heterosexual transmission (n=7), as well as surgical procedures and/or transfusions performed in an HIV-endemic region (n=3). For studies of cell-mediated immunity, patients were selected among participants to the St-Luc intravenous drug user cohort (CHUM-Hôpital St-Luc, Montreal, Canada) (n=7; HND, HTM, and PSL codes) or from the CMIS cohort (n=4; TVC codes). Clinical characteristics of subjects in this second study group are summarized in Table 1. Alanine transaminase (ALT) and aspartate transaminase (AST) levels were assayed on a Synchron LX20 system (Beckman Coulter, Palo Alto, Calif.). None of the subjects in either group had been treated with anti-HCV therapy at the time of the study.

HCV F Protein Gene Cloning and Mutagenesis Viral genomic RNA was isolated from 500 μl of serum obtained from a subject (TVC33) infected with HCV-1a, and was reverse transcribed and amplified using the QIAamp™ procedure (Qiagen, Mississauga, Ontario) with primers HCV 1a 1-16 (5′-GCC AGC CCC CTG ATG G-3′ [SEQ ID NO: 1]) and HCV 988-970 (5′GCC TCG TAC ACA ATA CTC G-3′ [SEQ ID NO: 2]) (Alpha DNA, Montreal, Canada). RT-PCR conditions were 50° C./30 min, 95° C./15 min denaturation, 40 cycles of 94° C./30 sec, 55° C./1 min, and 72° C./1 min, followed by a 72° C./10 min extension cycle, in a T3™ thermal cycler (Biometra, Goettingen, Germany). The amplicon was cloned into the Srf I site of pCRScript Amp SK+ (Stratagene Cloning Systems, La Jolla, Calif.) (pCore1a33). Core sequences were then modified by mutagenesis to a) force-shift translation into the (+2) reading frame; and b) lock translation by introducing three silent mutations within the frameshift-associated slippery-like sequence (Fmut8). This was done by first inserting the Aat II-Not I fragment from pCore1a33 into the pSC11ss vaccinia transfer vector [19] cleaved with Stu I and Not I (pSC11ssF16). pCore1a33 was reamplified using primers Fmut S Sal (5′-GAC CGT CGA CCA TGA GCA CGA ATC CTA AAC CTC AGA GGA AGA CCC CAA ACG TAA-3′ [SEQ ID NO: 3]) and Fmut AS Kpn (5′-AAG GGT ACC CGG GCT GAG CCC AGG TCC TGC CCT CGG G-3′ [SEQ ID NO: 4]). PCR conditions were 94° C./3 min, 25 cycles of 94° C./30 sec, 60-72° C./1 min, and 72° C./1 min, followed by 72° C./15 min extension, in a TGradient™ thermal cycler (Biometra). The amplicon was then cut with Sal I and Kpn I, and was shuffled into pSC11ssF16 to form pSC11ssFmut8. A truncated form of the F protein lacking the 11 first N-terminal amino acids shared with core (Fmut8Δ11) was similarly generated using primers HCV1a Sal I (5′-TCA AGT CGA CCC AAA CGT AAC ACC AAC CG-3′ [SEQ ID NO: 5]) and pCRS1 (5′-GGA AAC AGC TAT GAC CAT GAT TAC GCC AAG C-3′ [SEQ ID NO: 6]). PCR conditions were 25 cycles of 94° C./30 sec, 53° C./1 min, and 72° C./1 min, followed by 72° C./15 min extension. Lastly, Fmut8 and Fmut8Δ11 were also subcloned into Sal I-Not I-digested pET-30c and pET-30b, respectively (Novagen, Madison, Wis.), for inducible, N-terminal His-tagged expression in E. coli. The structure of all constructs was verified by automated DNA sequencing.

### Production of Recombinant F Protein

pET-30cFmut8 and pET-30bFmut8 μl were expressed in E. coli BL21 cells and F protein was purified under denaturing conditions on Ni-NTA His-Bind™ resin (Novagen), followed by preparative polyacrylamide gel electrophoresis using a Mini Prep™ cell (Bio-Rad Laboratories, Hercules, Calif.). The Fmut8Δ11 gene product was used to raise rabbit antiserum with no cross-reactivity against core protein. For mass spectrometry (MS), Coomassie-stained protein gel bands were rehydrated and trypsinized. Extracted peptides were then eluted from a nanoscale C18 reverse-phase HPLC capillary column and were subjected to electrospray ionization followed by MS using an LCQ DECA ion-trap mass spectrometer (ThermoFinnigan, San Jose, Calif.). Protein identity was assessed by sequence comparison with protein or translated nucleotide databases with the use of the SEQUEST program [20].

### F Protein ELISA

Antigen (2 μg/ml Ni-NTA-purified Fmut8Δ11) was incubated overnight at 4° C. in flat-bottom 96-well polystyrene plates in 100 μl of 0.1 M NaHCO₃ pH 8.6. Plates were blocked for 2 h/4° C. with 200 μl per well of 5 mg/ml bovine serum albumin (BSA) in 100 mM NaHCO₃ pH 8.6, and were washed 6 times with 10 mM tris base, 150 mM NaCl, 0.05% Tween-20 pH 7.4 (1×TBST). Dilutions of patient's sera (1/50 to 1/6400 in 100 μl 1×TBST) were then added and incubated for 2 h at room temperature with gentle shaking. Wells were washed 6 times with 1×TBST and 100 μl/well of alkaline-phosphatase-coupled anti-human IgG antibody (1/3000; Sigma, Saint Louis, Mo.) or anti-rabbit antibody (1/1000; Biosys, Compiegne, France) was added in 1×TBST+5 mg/ml BSA. After 1 hour incubation at room temperature with gentle agitation, wells were washed 6 times and antibody binding was revealed with 50 mM NaHCO₃, 1 mM MgCl₂, and 1 mg/ml p-nitrophenyl-phosphate. After 90 min, optical density was measured at 410 nm in a MR7000 spectrophotometer (Dynatec, Chantilly, Va.). Rabbit antisera raised against Fmut8Δ11 or purified core protein were used as positive and negative controls.

### Preparation of Recombinant Vaccinia Virus

CV1 cells, maintained in Dulbecco's minimal essential medium (DMEM) supplemented with 10% fetal bovine serum (FBS) (Invitrogen, Burlington, Canada), were infected with the Western Reserve (WR) strain of vaccinia virus and were then transfected with pSC11ssFmut8 using CaCl₂ precipitation. Progeny virus was extracted by 3 freeze-thaw cycles, treatment with 0.25 mg/ml trypsin (Worthington, Lakewood, N.J.) and sonication, followed by 3 rounds of plaque selection on HuTK-143B cells incubated in 2% w/v low melting point agarose (Invitrogen) supplemented with 2×DMEM, 0.05 mg/ml neutral red, 80 μM bromodeoxyuridine (BrdU), and 400 μM 5′-bromo-4-chloro-3-indolyl-β-D-galactopyranoside (X-Gal) [19]. Identity and structure of plaque-purified vaccinia recombinants were verified by PCR and automated DNA sequencing.

### Cytotoxic T Lymphocyte Assays

To serve as targets in cellular cytotoxicity assays, autologous B-lymphoblastoid cell lines (BLCL) were derived from each of the study subjects by incubating patient peripheral blood mononuclear cells (PBMC) isolated on a Ficoll gradient (Amersham Biosciences, Mississauga, Canada) with supernatant from the B95-8 Epstein-Barr virus (EBV)-infected cell line in presence of 1 μM cyclosporine A (Sandoz, Vienna, Austria) [21]. For limiting dilution analysis (LDA), serial dilutions (500 cells per well-7 cells per well) of patient PBMC were replica-plated onto wells containing 200,000 irradiated (3000 rads) allogeneic feeders cells, and were cultivated for 21 days in RPMI media supplemented with 10% FBS, 1 μg/ml phytohemagglutinin (PHA) (Sigma) and 80 units/ml recombinant interleukin-2 (IL-2) (Hoffman-La Roche, Nutley, N.J.; obtained through the NIH AIDS Research and Reference Reagents Program). Autologous BLCL, infected at a MOI of 10 with specific vaccinia recombinants, were labeled with ⁵¹Cr-sodium chromate (Amersham Biosciences) and the cytotoxic activity of cultured effectors was tested following a standard 5 h incubation [22]. Wild type vaccinia WR and vaccinia SC59 NNRd expressing amino acid residues 364-1618 (E2, p7, NS2, and NS3) of HCV-1a [23] were used as controls in these experiments. Percent specific lysis was defined as 100×(test release-spontaneous release)/(total release-spontaneous release), with significance threshold set at 2.67 standard deviations above negative control values. CTL precursor frequencies and 95% confidence intervals were computed using software described in Walter J B, et al. (1997, Int Immunol 9: 451-459).

### Peptide Binding Assay

37 overlapping peptides (15 amino acid residues with 10 residue overlaps) derived from F protein and 5 additional 15-mer peptides corresponding to alternative N-terminal frameshift products of the core protein sequence were synthesized using Fmoc chemistry (SynPep, Dublin, Calif.) based on the sequence of HCV-1a F protein (GenBank accession n° M62321) [24], and were resolubilized in 25% w/v dimethyl sulfoxyde (DMSO) in 1×HBSS. Peptides were diluted to 30, 100, and 200 μg/ml final concentration in DMEM supplemented with 3% w/v FBS, and were incubated with 100,000 serum-depleted (3% FBS for 24 h) T2 cells [25] for 16 h at 37° C. in 5% CO₂. T2 cells were then stained (30 min at room temperature) for MHC class I molecules using fluorescein isothiocyanate (FITC)-conjugated W6/32 monoclonal antibody (Sigma, Saint Louis, Mo.) and were analyzed on a FACSCalibur™ flow cytometer (Becton-Dickinson Immunocytometry Systems, La Jolla, Calif.) with live gating based on forward and side scatter.

### Example 2

### Analysis of F Protein-Specific Humoral Immune Responses

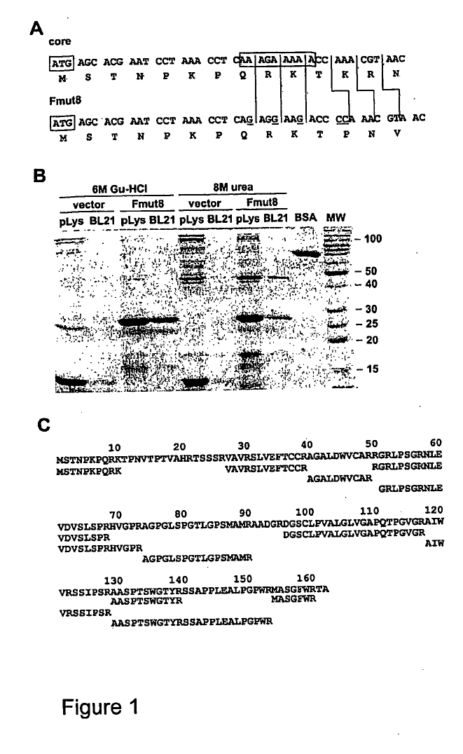

Five synonymous nucleotide substitutions were introduced in the putative frameshift-associated slippery-like sequence to force expression of HCV-1a F protein in the absence of core gene product (FIG. 1A). This recombinant protein (FIG. 1B) and a truncated form lacking the first 11 amino acid residues shared with core (data not shown) were expressed in E. coli and purified by nickel chelation chromatography. Because multiple translational recoding events were reported to occur when F protein was produced in different expression systems [13, 26, 27], the amino acid sequences of Fmut8 and Fmut8Δ11 were confirmed using MS, with peptide coverage of 87.0% and 68.2% by amino acid count for Fmut8 and Fmut8Δ11, respectively (FIG. 1C and data not shown). Purified Fmut8Δ11 was used in ELISA to measure reactivity against F protein in sera derived from 39 patients infected with various HCV subtypes, including HCV-1a (n=20), HCV-1b (n=9), HCV-3a (n=7), HCV-4-a (n=1), HCV-4c (n=1), and HCV-5a (n=1). Of these patients, 25 were also coinfected with HIV-1. Rabbit antisera raised against Fmut8Δ11 or purified core protein (described in Majeau N, et al. (2004) J. Gen. Virol. 85: 971-981.) were used as positive and negative controls. Levels of ALT and AST were significantly higher in coinfected patients than in patients infected with HCV alone (p=0.0464 and p=0.00542, respectively, Student's t test). Plasma HCV load was also larger in coinfected subjects, but this difference was not statistically significant (p=0.0778, Student's t test).

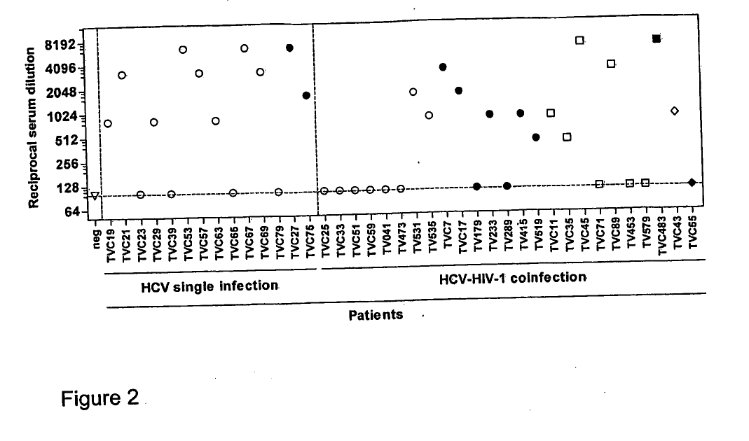

Overall, serum samples from 23 of 39 HCV-infected patients (59.0%) had positive reactivity to Fmut8Δ11 at dilutions of 1:400 or greater (FIG. 2). These include 10 of 20 patients (50.0%) infected with HCV-1a, 7 of 9 patients (77.8%) infected with HCV-1b, 4 of 7 patients (57.1%) infected with HCV-3a, 2 of 2 patients (100%) infected with HCV-4a or 4c, and 0 of 1 patient (0.00%) infected with HCV-5a. Humoral responses directed against F protein were only detected in HCV infected subjects (FIG. 2). Plasma HCV viral load, ALT and AST levels were not significantly different in subject with detectable versus undetectable anti-F antibody response (p=0.137, 0.112, and 0.105, respectively, Student's t test). Furthermore, there was no correlation in linear regression analysis between titers of anti-F antisera and HCV viral load, ALT, or AST (r²=0.124, 0.134, and 0.125, respectively).

Furthermore, the proportion of patients coinfected with HCV and HIV-1 who had detectable antibody responses to F protein (13 of 25 subjects; 52.0%) was not significantly different from that observed in subjects infected with HCV alone (10 of 14 subjects; 71.4%) (p=0.317, Fisher's exact test), and was similar to that reported in a recent survey of HCV-infected subjects [17]. HCV subtype cross-reactive recognition of F protein was also observed in sera from coinfected patients (FIG. 2). When analysis was restricted to patients with detectable anti-F antibody responses, ELISA titers were not significantly different in coinfected patients (n=13) than those measured in sera from subjects infected with HCV alone (n=10) (p=0.170, Mann-Whitney U test) (FIG. 2). F protein-specific antibody responses were detected in coinfected patients with CD4 counts ranging from 33 cells/mm³ to 1287 cells/mm³ (FIG. 2C). However, there was no significant correlation between anti-F antibody titer and CD4 cell counts (r 2=0.146) (FIG. 2B), though the correlation coefficient improved (r²=0.205) and bordered statistical significance when subjects with CD4 counts<200 cells/mm³ were excluded from the analysis. There was no significant correlation between anti-F antibody titer and HIV-1 viral load (r²=0.00172). Finally, cross-reactive HCV subtype recognition of F protein was observed in sera from coinfected patients (FIG. 2). ELISA results were corroborated by western blot. Overall, these data indicate that coinfected patients can mount immunoglobulin responses against HCV F protein, and that these responses are cross-reactive between HCV subtypes.

### Example 3

### Analysis of F Protein-Specific CTL Activity

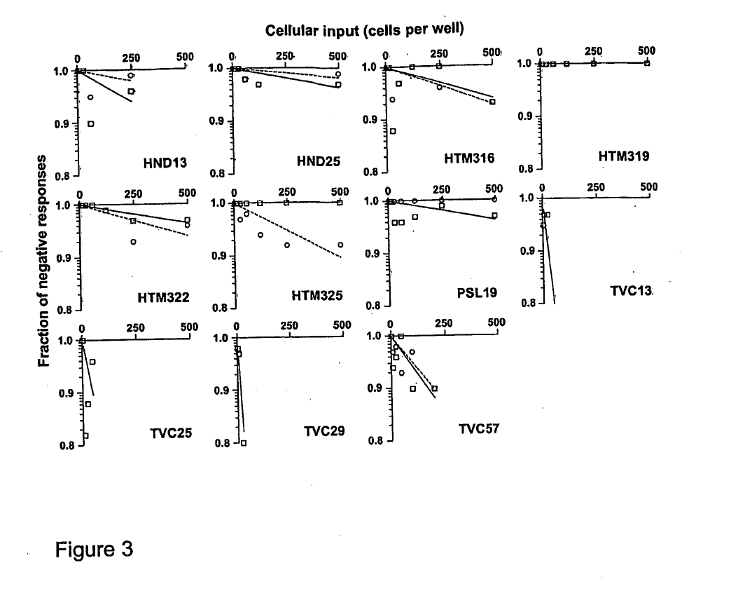

T cell microcultures were derived from peripheral blood mononuclear cells (PBMC) samples obtained from 11 HCV-infected patients, including 9 subjects coinfected with HCV and HIV-1. Vaccinia-Fmut8 recombinant virus was generated and used in ⁵¹Cr-release LDA to test F protein-specific CTL activity (Table 1; FIG. 3). Overall, CTL precursors were detected in 9 of 11 HCV-infected subjects (81.8%), with precursor frequencies ranging from 1/177 to 1/13372 T cells. These include patients infected with HCV-1a (n=6), HCV-2a (n=1) and HCV-3a (n=2), indicative of cross-reactive recognition of F protein by CTL between HCV subtypes. While others have reported production of IFN-γ and IL-10 following in vitro stimulation of T cells with F protein-derived peptides [16], our results represent the first direct evidence of cell-mediated cytotoxic activity directed against HCV F protein in HCV-infected subjects. CTL precursors were also detected in 7 of 9 patients (77.8%) co-infected with HIV-1 (mean CD4 T cell counts: 519 cells/μl; mean HIV-1 viral load: 3.46 log RNA copies/ml. Table 1). CTL precursors that recognized other HCV proteins (residues 364-1618, ie. E2, p7, NS2, NS3, but not core or F protein) were also observed in 6 of 8 subjects (75%), with precursor frequencies between 1/1608 and 1/12188, not significantly different from those observed with F protein (p=0.529, Mann-Whitney U test). Patient HTM319 did not exhibit cytotoxic activity against either of the targets tested, while patients HTM325 and PSL19 had discordant responses to F protein and p364-1618 (Table 1; FIG. 3). Finally, there was no correlation between F protein-specific CTL precursor frequencies and patient CD4 cell counts, HIV-1 or HCV viral load.

### Example 4

### Analysis of F Protein Peptide Binding

Interaction of antigenic peptides with class I major histocompatibility complex (MHC) molecules is a prerequisite for their recognition by host cytotoxic T lymphocytes (CTL). The T2 binding assay was therefore performed, in which exogenously-supplied peptides are assessed for their ability to rescue cell-surface expression of CMH-I molecule HLA-A2 in the TAP peptide transporter-deficient cell line T2 [25].

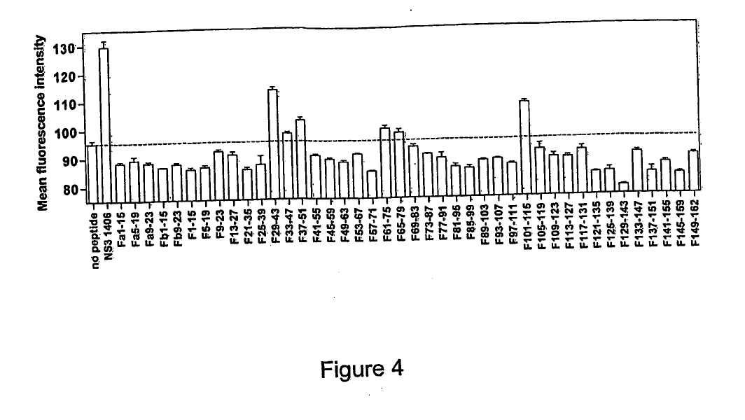

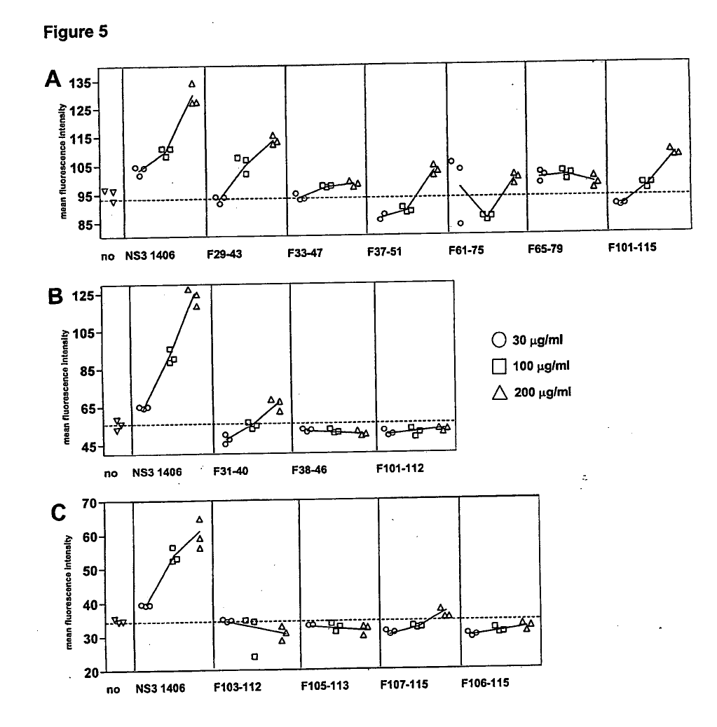

Forty-two overlapping 15-mer peptides corresponding to HCV-1a F protein sequence were tested for interaction with HLA-A*0201 molecules using the T2 peptide binding assay [25]. Specifically, thirty-seven overlapping peptides (15 amino acid residues with 11 residues overlaps) derived from F protein and 5 additional 15-mer peptides corresponding to alternative N-terminal frameshift products of the core protein sequence were synthesized using Fmoc chemistry based on the sequence of HCV-1a ARFP (GenBank accession n° M62321) [24], and peptide binding was assessed as described in Example 1. The HCV-1 NS3 1406-1415 peptide (KLVALGINAV [SEQ ID NO: 13]), a well-characterized HLA-A*0201 restricted T-cell epitope [28], was used as positive control. Mean fluorescence intensity following class I MHC staining by the W6/32 monoclonal antibody was used as the readout. The threshold level was set using no peptide controls (hatched lines in FIGS. 4 and 5).

While the efficacy of individual F protein-derived peptides to promote HLA-A*0201 expression at the cell surface varied considerably, six 15-mers stood out as high binders in initial screening experiments (F29-43, F33-47, F37-51, F61-75, F65-79, and F101-115) (200 μg/ml, FIG. 4). Of those, only peptides F29-43 (VRSLVEFTCCRAGAL [SEQ ID NO: 14]), F37-51 (CCRAGALDWVCARRE [SEQ ID NO: 21]), and F101-115 (PVALGLAGAPQTPGV [SEQ ID NO: 29]) clearly mediated a dose-dependent increase in MHC class I expression at the surface of T2 cells (FIG. 5A). Peak fluorescence obtained with peptides F29-43, F37-51, and F101-115 respectively reached 87.6%, 79.2%, and 83.2% of the levels obtained with NS3 1406-1415 (FIG. 5A).

A further epitope within peptide F29-43 (VRSLVEFTCCRAGAL [SEQ ID NO: 14; SEQ ID NO: 15 underlined]) was analyzed herein, and it was determined that exposure of T2 cells to peptide F31-40 (SLVEFTCCRA; SEQ ID NO: 15) clearly resulted in a dose-dependent increase in cell-surface expression of MHC class I (FIG. 5B). This is strongly supportive of the fact that F31-40 could be recognized by CTL in HLA-A*0201 subjects infected with HCV-1a. Analysis of this peptide using the BIMAS web tool (http://bimas.dcrt.nih.gov/molbio/hla_bind/)[34]) suggested it to be a predicted HLA-A*0201-restricted epitope, with a predicted MHC-peptide dissociation time of 20.4 seconds (Table 2).

Analysis of a further epitope within peptide F101-115 (PVALGLAGAPQTPGV) [SEQ ID NO: 29; SEQ ID NO: 30 underlined], indicated that this peptide (F103-112) was unable to rescue HLA-A2 expression at the surface of T2 cells (FIG. 5C). BIMAS analysis of this peptide suggested it to be a predicted HLA-A*0201-restricted epitope, with a predicted dissociation time of 7.45 seconds (Table 2).

Further epitopes located within F37-51 and F101-115 were analyzed, including CRAGALDWV (F38-46; SEQ ID NO: 22), CCRAGALDWV (F37-46; SEQ ID NO: 23), GLAGAPQTP (F105-113; SEQ ID NO: 31), AGAPQTPGV (F107-115; SEQ ID NO: 32), and LAGAPQTPGV (F106-115; SEQ ID NO: 33). Of these, F107-115 showed a dose-dependent capacity to rescue A2 expression at the surface of T2 cells (significant A2 expression was observed at a peptide concentration of 200 μg/ml) (Table 2; FIG. 5C). Analysis of these peptides (F38-46; F37-46; F105-113; F107-115 and F106-115) using the SYFPEITHI algorithm (http://www.syfpeithi.de) [35] suggested these to be potential rHLA-A*0201-restricted epitopes (Table 2).

Therefore, the data presented herein indicates that F31-40 and F107-115, which are found within F29-43 and F101-115, respectively, represent minimal A2-restricted CTL epitopes.

Within our panel, SLVEFTCCRA (F31-40; SEQ ID NO: 15) is only represented in a single peptide (ie. F29-43). It lies within the region of ARFP which is the most conserved between HCV subtypes (Table 3), indicating that they could display antigenic cross-reactivity. In addition, both HLA-A*0201 anchor residues (P2 and P9) are fully conserved across all HCV genotypes examined (Table 3). Peptide F37-51 is likewise highly conserved, with 9 of 15 (60%) amino-acid positions fully identical in all subtypes (Table 11). In contrast, AGAPQTPGV (SEQ ID NO: 32) resides within a section of ARFP which is highly variable between genotypes, and this variability extends to anchor residues as well. This suggests that AGAPQTPGV (SEQ ID NO: 32) could be HCV-1 subtype-specific and that it might not be equally recognized in subjects infected with other HCV strains.

Use of the SYFPEITHI algorithm also suggested the potential HLA-A*0201-restricted epitope (CRAGALDWV [SEQ ID NO: 22]) located within peptides F33-47 and F37-51. The lack of a clear dose-response with F33-47 as compared to F37-51 (FIG. 5A) could be due to differential peptide processing by T2 cells.

Overall, several peptide sequences derived from HCV-1a ARFP were capable of binding HLA-A*0201 in cell culture and therefore correspond to novel, HLA-A*0201-restricted CTL epitopes. These epitopes are different from those previously reported for HCV-1b [16], with SLVEFTCCRA (F31-40; SEQ ID NO: 15) and F37-51 positioned upstream of the sequence corresponding to the 99 amino acid synthetic peptide used by this group [16; 36].

TABLE 1
Cytotoxic activity directed against F protein in patients infected with HCV

| | | | | CD4 | HIV load | HCV | | | | | 95% | | 95% |
|---|---|---|---|---|---|---|---|---|---|---|---|---|---|
| | | | | count | (log | load | | | | CTLp | confidence | CTLp | confidence |
| | | | | (cells/ | copies/ | (log | HCV | ALT | AST | Fmut8 | interval | NNRd | interval |
| Subjects | Age | Gender | HIV | μl) | ml) | IU/ml) | Genotype | (U/ml) | (U/ml) | (1/x) | (1/x) | (1/x) | (1/x) |
| HND 013 | 40.0 | M | + | 480 | 4.46 | 7.39 | 3a | 306 | 135 | 3671 | 1523-8848 | 9311 | 2395-37298 |
| HND 025 | 43.9 | M | + | 580 | 5.39 | 6.69 | 3a | 60 | 53 | 9702 | 4027-23372 | 12188 | 4559-32587 |
| HTM 316 | 45.3 | M | + | 771 | 4.08 | 6.39 | 1a | 76 | 60 | 7697 | 3846-15404 | 6122 | 3291-11389 |
| HTM 319 | 43.7 | M | + | 364 | 2.00 | 7.33 | 1a | 223 | 158 | nd | nd | nd | nd |
| HTM 322 | 47.0 | M | + | 344 | <1.70 | 7.26 | 2a | 45 | 43 | 13772 | 5565-32131 | 6612 | 3300-13246 |
| HTM 325 | 44.9 | M | + | 604 | 1.72 | 6.55 | 3a | 127 | 137 | nd | nd | 3667 | 2306-5833 |
| PSL 019 | 43.9 | M | + | 370 | 4.79 | 7.28 | 1a | 22 | 37 | 8278 | 4127-16604 | nd | nd |
| TVC13 | 20.1 | F | + | 598 | 4.69 | 7.37 | 1a | 65 | 66 | 238 | 132-430 | nt | nt |
| TVC25 | 31.9 | F | + | 561 | 2.35 | 6.82 | 1a | 18 | 24 | 221 | 124-392 | nt | nt |
| TVC29 | 24.2 | F | − | nt | nt | 4.61 | 1a | 47 | 20 | 177 | 84-369 | nt | nt |
| TVC57 | 23.4 | F | − | nt | nt | 6.56 | 1a | 30 | 25 | 1422 | 894-2260 | 1608 | 983-2631 |

CTL precursor frequencies and 95% confidence intervals were derived as described under Materials and Methods.
nd: none detected.
nt: not tested.

TABLE 2
uz,4/29 Summary of ARFP peptide binding assays

| | | | | | |
|---|---|---|---|---|---|
| | SEQ | | A2 | | SYFPE- |
| Pep- | ID | Amino acid | bind- | Bimas² | ITHI³ |
| tide | NO: | sequence | ing¹ | t_{½} | score |

| | | | | | |
|---|---|---|---|---|---|
| NS3 | 13 | KLVALGINAV | + | 559.894 | 27 |
| 1406 | | | | | |
| F29- | 14 | VRSLVEFTCCRAGAL | + | na | na |
| 43 | | | | | |
| F31- | 15 | SLVEFTCCRA | + | 20.369 | 18 |
| 40 | | | | | |
| F37- | 21 | CCRAGALDWVCARRE | + | na | na |
| 51 | | | | | |
| F38- | 22 | CRAGALDWV | − | 0.060 | 18 |
| 46 | | | | | |
| F37- | 23 | CCRAGALDWV | − | 0.608 | 14 |
| 46 | | | | | |
| F101- | 29 | PVALGLAGAPQTPGV | + | na | na |
| 115 | | | | | |
| F103- | 30 | ALGLAGAPQT | − | 7.452 | 17 |
| 112 | | | | | |
| F105- | 31 | GLAGAPQTP | − | 0.015 | 17 |
| 113 | | | | | |
| F107- | 32 | AGAPQTPGV | + | 0.454 | 20 |
| 115 | | | | | |
| F106- | 33 | LAGAPQTPGV | − | 1.642 | 18 |
| 115 | | | | | |

¹Peptide treatment leading to a dose-dependent increase in the levels of expression of HLA-A2 at the surface of T2 cells (T2 peptide binding assay).
²See ref. 34; http://bimas.dcrt.nih.gov/molbio/hla_bind/.
³See ref. 35; http://www.syfpeithi.de.

TABLE 3

Conservation of potential HLA-A*0201-restricted
epitopes located within F protein peptides
F29-43, F37-51, and F101-115 between HCV
subtypes.

| HCV | | | |
|---|---|---|---|
| subtype | F29-43 | F37-51 | F101-115 |
| 1a [M62321] | SLVEFTCCRA | CRAGALDWV | ALGLAGAPQT |
| | (SEQ ID NO: | (SEQ ID NO: | (SEQ ID NO: |
| | 15) | 22) | 30) |
| 2a [D00944] | SLAEYTCCRA | CRAGAPGWV | VPVPLGAPMT |
| | (SEQ ID NO: | (SEQ ID NO: | (SEQ ID NO: |
| | 16) | 24) | 34) |
| 3a [D17763] | SLVEYTCCRA | CRAGAHDWV | APVHPGAQMT |
| | (SEQ ID NO: | (SEQ ID NO: | (SEQ ID NO: |
| | 17) | 25) | 35) |
| 4a [Y11604] | SLAEFTCCRA | CRAGAPDWV | ALDRLGAQMI |
| | (SEQ ID NO: | (SEQ ID NO: | (SEQ ID NO: |
| | 18) | 26) | 36) |
| 5a [Y13184] | SLVEFTCCRA | CRAGALNWV | ALGLIGAPMT |
| | (SEQ ID NO: | (SEQ ID NO: | (SEQ ID NO: |
| | 19) | 27) | 37) |
| 6a [Y12083] | SLAEFTCCRA | CRARAPGWV | APGHTGAPMT |
| | (SEQ ID NO: | (SEQ ID NO: | (SEQ ID NO: |
| | 20) | 28) | 38) |

GenBank accession numbers are shown between brackets. Conserved HLA-A*0201 anchor residues are in bold.

### Example 4

### Induction of ARFP-Specific Immune Responses in Mice

A series of experiments were performed to demonstrate whether recombinant ARFP (i.e. Fmut8Δ11 form produced in Escherichia coli and purified by nickel chelation chromatography and preparative electrophoresis) was able to induce significant and specific immune responses in mice. In a first set of experiments, induction of ARFP-specific humoral (i.e. antibody) immune responses was tested as follows.

### ARFP-Specific Humoral Immune Responses.

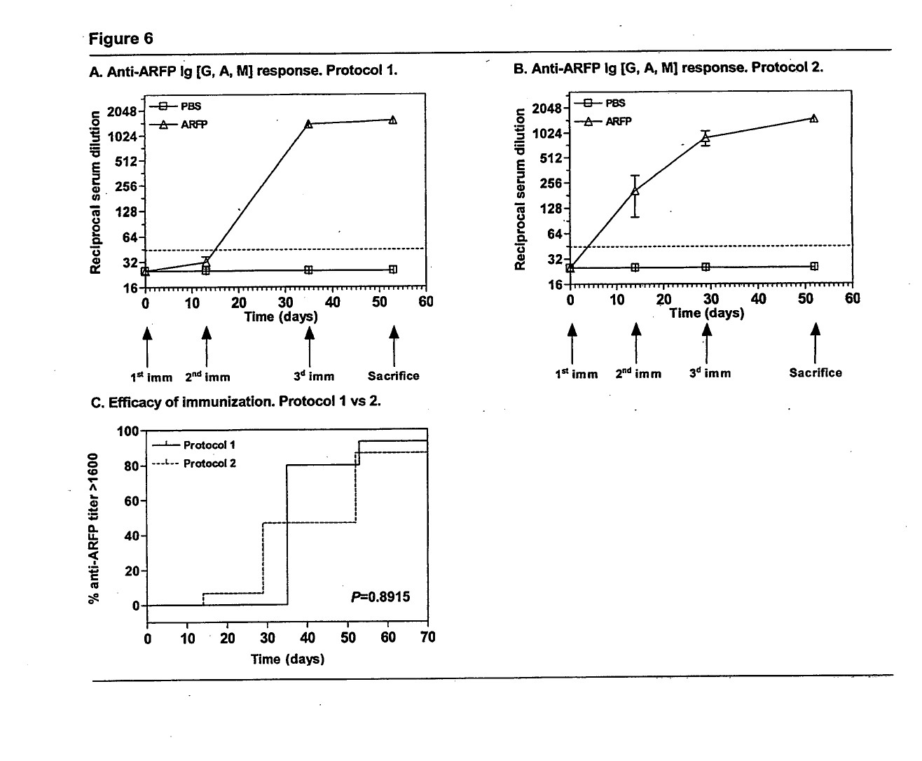

Protocol 1. On day 0, 15 5-week old female C57Bl/6 mice were injected subcutaneously (back) with 10 μg (50111) purified Fmut8Δ11 protein emulsified in 50 μl incomplete Freund's adjuvant (100 μl total). Ten 5-week old female C57Bl/6 mice were injected with 100 μl phosphate-buffered saline and were used as controls. An identical immunization procedure was repeated on day 13, and again on day 35. 200 μl of blood was obtained by maxillary bleeding prior to each immunization (days 0, 13, and 35). Mice were sacrificed by CO₂ asphyxiation and bled by cardiac puncture on day 53. All blood samples were centrifuged and sera was collected and frozen at −80° C. until used. Total immunoglobulin (Ig) responses (IgA, IgM, and IgG) were tested by enzyme-linked immuno-sorbent assay (ELISA) as described in Example 1, except that alkaline phosphatase-conjugated anti-mouse polyvalent immunoglobulins (G, A, M) (A-0162, Sigma, St. Louis, Mo.) were used instead of alkaline phosphatase-conjugated monoclonal anti-human IgG (A-2064, Sigma). Anti-ARFP antibody binding was revealed with 50 mM sodium bicarbonate, 1 mM magnesium chloride, and 1 mg/ml p-nitrophenyl-phosphate. Optical density was measured at 410 nm (Table 4). ELISA controls included: a) no antigen, no mouse antiserum, no secondary antibody; b) no antigen, mouse antiserum, secondary antibody; c) ARFP, mouse antiserum, no secondary antibody; d) ARFP, no mouse antiserum, secondary antibody; e) ARFP, anti-ARFP rabbit antiserum [prepared in the studies described herein using Fmut8Δ11 as the immunogen], anti-rabbit secondary antibody (positive control); and f) ARFP, mouse antiserum, anti-human secondary antibody (Table 6). The ELISA threshold value was set at 3 times the OD₄₁₀ measured in the negative control with the highest readout (highlighted in Table 6). Results were expressed as mean+/−standard deviation of the lowest reciprocal serum dilution showing an OD₄₁₀ value greater than the threshold value. Results showed that anti-ARFP Ig titer reached 1400+/−419.5 on day 35, and 1547+/−199.6 on day 53 (Table 4, FIG. 6A). Anti-ARFP titers were uniformly negative in all control mice (Table 5, FIG. 6A). On day 35, 12 of 15 mice (80%) immunized with recombinant ARFP had reached an anti-ARFP Ig titer of >1600, and this proportion reached 93.3% (14 of 15 mice) on day 53 (Table 4, FIG. 6C).
Protocol 2. On day 0, 15 5-week old female C57Bl/6 mice were injected subcutaneously with 10 μg (50 μl) purified Fmut8Δ11 protein emulsified in 50 μl incomplete Freund's adjuvant (100 μl total). Fourteen 5-week old female C57Bl/6 mice were injected with 100 μl phosphate-buffered saline and were used as controls. An identical immunization procedure was repeated on day 14, and again on day 29. 200 μl of blood was obtained by maxillary bleeding prior to each immunization (days 0, 14, and 29). Mice were sacrificed by CO₂ asphyxiation and bled by cardiac puncture on day 52. Isolation of serum and ELISA were performed as above. ELISA controls included: a) ARFP, mouse antiserum, no secondary antibody; b) ARFP, no mouse antiserum, secondary antibody; c) ARFP, anti-ARFP rabbit antiserum [prepared in the studies described herein using Fmut8Δ11 as the immunogen], anti-rabbit secondary antibody (positive control); and d) ARFP, mouse antiserum, anti-human secondary antibody (Table 9). The ELISA threshold value was set at 3 times the OD₄₁₀ measured in the negative control with the highest readout (highlighted in Table 9). The determination of detection threshold and anti-ARFP Ig titers was performed as above. Results showed that anti-ARFP Ig titer reached 883.3+/−694.7 on day 29, and 1493+/−272.0 on day 52 (Table 7, FIG. 6B). As before, anti-ARFP titers were uniformly negative in all control mice (Table 8, FIG. 6B). On day 29, 7 of 15 mice (46.7%) immunized with recombinant ARFP had reached an anti-ARFP Ig titer of >1600, and this proportion reached 86.7% (13 of 15 mice) on day 52 (Table 7, FIG. 6C).

The kinetics of antibody responses in protocols 1 and 2 were similar but not overlapping, as ARFP-specific Ig titers measured before the 2^{nd} immunization were higher in protocol 2 (190.0+/−401.7) than in protocol 1 (10.00+/−27.08). However, this difference was not statistically significant (p=0.116, Student's t test). Similarly, the proportion of animals having reached detectable anti-ARFP Ig titers before the 2^{nd} immunization was not significantly different between protocols 1 and 2 (p=0.215, Fisher's exact test) (Tables 4 and 7). Finally, when acquisition of ARFP-specific Ig titers>1600 was used as primary outcome of immunization in Kaplan-Meier analysis (FIG. 1C), protocols 1 and 2 were not significantly different in terms of efficacy (p=0.892, log rank test). Therefore, results obtained in protocols 1 and 2 are fully consistent with one another. Taken together, results of these two protocols indicate that immunization with ARFP (Fmut8Δ11) results in the generation of high level ARFP-specific Ig responses in C57Bl/6 mice. For example, such an immunization may be effected via a regimen comprised of three subcutaneous immunizations with 10 μg purified ARFP (Fmut8Δ11) given at 2 weeks intervals.

TABLE 4
Protocol 1: ARFP immunization, ELISA results

| | Mouse | | | | | | | |
|---|---|---|---|---|---|---|---|---|
| Dilution | 31 | 32 | 33 | 34 | 35 | 36 | 37 | 38 |

1^{st} immunization (day 0)

| | | | | | | | | |
|---|---|---|---|---|---|---|---|---|
| 1/50 | 0.166 | 0.236 | 0.243 | 0.247 | 0.208 | 0.206 | 0.205 | 0.217 |
| 1/100 | 0.115 | 0.161 | 0.161 | 0.145 | 0.134 | 0.132 | 0.142 | 0.127 |
| 1/200 | 0.123 | 0.150 | 0.128 | 0.136 | 0.114 | 0.111 | 0.121 | 0.113 |
| 1/400 | 0.132 | 0.164 | 0.114 | 0.118 | 0.116 | 0.121 | 0.133 | 0.115 |
| 1/800 | 0.119 | 0.138 | 0.113 | 0.118 | 0.115 | 0.117 | 0.128 | 0.115 |
| 1/1600 | 0.138 | 0.122 | 0.109 | 0.110 | 0.109 | 0.116 | 0.124 | 0.108 |
| Titer | 0 | 0 | 0 | 0 | 0 | 0 | 0 | 0 |

2^{nd} immunization (day 13)

| | | | | | | | | |
|---|---|---|---|---|---|---|---|---|
| 1/50 | 0.732 | 0.272 | 0.750 | 0.293 | 0.231 | 0.965 | 0.137 | 0.916 |
| 1/100 | 0.306 | 0.117 | 0.197 | 0.157 | 0.138 | 0.890 | 0.113 | 0.742 |
| 1/200 | 0.165 | 0.113 | 0.166 | 0.127 | 0.160 | 0.815 | 0.108 | 0.441 |
| 1/400 | 0.147 | 0.110 | 0.154 | 0.121 | 0.117 | 0.677 | 0.110 | 0.328 |
| 1/800 | 0.124 | 0.106 | 0.137 | 0.123 | 0.125 | 0.454 | 0.105 | 0.219 |
| 1/1600 | 0.120 | 0.118 | 0.119 | 0.118 | 0.120 | 0.590 | 0.104 | 0.171 |
| Titer | 0 | 0 | 0 | 0 | 0 | 100 | 0 | 50 |

3^{d} immunization (day 35)

| | | | | | | | | |
|---|---|---|---|---|---|---|---|---|
| 1/50 | 3.652 | 3.551 | 3.142 | 3.623 | 3.509 | 3.334 | 3.530 | 3.887 |
| 1/100 | 3.642 | 2.683 | 3.700 | 2.841 | 3.563 | 3.625 | 3.306 | >4.000 |
| 1/200 | 3.616 | 2.046 | 3.630 | 2.088 | 3.556 | 3.596 | 2.436 | 3.957 |
| 1/400 | 3.569 | 1.681 | 3.296 | 1.522 | 3.465 | 3.590 | 1.859 | 3.799 |
| 1/800 | 3.434 | 1.072 | 3.166 | 0.937 | 3.213 | 3.438 | 1.297 | 3.425 |
| 1/1600 | 3.521 | 0.641 | 3.190 | 0.590 | 3.181 | 3.455 | 0.931 | 3.139 |
| Titer | >1600 | 800 | >1600 | 800 | >1600 | >1600 | >1600 | >1600 |

Sacrifice (day 53)

| | | | | | | | | |
|---|---|---|---|---|---|---|---|---|
| 1/50 | 2.023 | 2.008 | 1.658 | 1.054 | 1.709 | 1.411 | 1.314 | 1.736 |
| 1/100 | 2.028 | 1.542 | 1.830 | 1.646 | 1.839 | 1.829 | 1.483 | 2.139 |
| 1/200 | 2.202 | 2.602 | 1.876 | 1.837 | 1.805 | 2.091 | 1.948 | 2.168 |
| 1/400 | 2.376 | 2.547 | 1.889 | 1.913 | 1.776 | 2.225 | 1.925 | 2.356 |
| 1/800 | 2.490 | 1.574 | 1.874 | 1.814 | 1.617 | 2.117 | 1.671 | 1.664 |
| 1/1600 | 2.209 | 1.970 | 2.157 | 1.987 | 1.674 | 2.269 | 1.574 | 1.671 |
| Titer | >1600 | >1600 | >1600 | >1600 | >1600 | >1600 | >1600 | >1600 |

| | Mouse | | | | | | |
|---|---|---|---|---|---|---|---|
| Dilution | 39 | 40 | 41 | 42 | 43 | 44 | 45 |
| 1^{st} immunization (day 0) | | | | | | | |
| 1/50 | 0.211 | 0.209 | 0.199 | 0.218 | 0.323 | 0.278 | 0.229 |
| 1/100 | 0.128 | 0.115 | 0.174 | 0.208 | 0.186 | 0.171 | 0.152 |
| 1/200 | 0.113 | 0.104 | 0.117 | 0.136 | 0.164 | 0.144 | 0.140 |
| 1/400 | 0.104 | 0.110 | 0.120 | 0.128 | 0.191 | 0.138 | 0.126 |
| 1/800 | 0.108 | 0.103 | 0.105 | 0.123 | 0.134 | 0.137 | 0.114 |
| 1/1600 | 0.106 | 0.107 | 0.105 | 0.119 | 0.129 | 0.127 | 0.114 |
| Titer | 0 | 0 | 0 | 0 | 0 | 0 | 0 |
| 2^{nd} immunization (day 13) | | | | | | | |
| 1/50 | 0.132 | 0.13 | 0.186 | 0.049 | 0.046 | 0.048 | 0.045 |
| 1/100 | 0.112 | 0.116 | 0.105 | 0.045 | 0.053 | 0.048 | 0.046 |
| 1/200 | 0.107 | 0.111 | 0.107 | 0.102 | 0.798 | 0.116 | 0.123 |
| 1/400 | 0.107 | 0.105 | 0.115 | 0.101 | 0.279 | 0.103 | 0.120 |
| 1/800 | 0.099 | 0.106 | 0.106 | 0.097 | 0.273 | 0.106 | 0.106 |
| 1/1600 | 0.105 | 0.108 | 0.103 | 0.096 | 0.223 | 0.110 | 0.110 |
| Titer | 0 | 0 | 0 | 0 | 0 | 0 | 0 |
| 3^{d} immunization (day 35) | | | | | | | |
| 1/50 | 3.464 | 3.796 | 1.628 | 1.467 | 2.140 | 2.336 | 2.453 |
| 1/100 | 3.808 | >4.000 | 1.093 | 1.941 | 2.487 | 1.264 | 2.058 |
| 1/200 | 3.541 | >4.000 | 3.494 | 2.657 | 1.722 | 0.993 | 1.772 |
| 1/400 | 3.582 | 3.896 | 3.238 | 2.397 | 1.929 | 0.725 | 1.607 |
| 1/800 | 3.053 | 2.776 | 2.442 | 2.015 | 1.185 | 0.508 | 1.517 |
| 1/1600 | 2.783 | 1.989 | 1.713 | 1.578 | 0.957 | 0.408 | 1.453 |
| Titer | >1600 | >1600 | >1600 | >1600 | >1600 | 200 | >1600 |
| Sacrifice (day 53) | | | | | | | |
| 1/50 | 2.224 | 1.741 | 1.006 | 1.424 | 1.613 | 1.434 | 1.373 |
| 1/100 | 1.904 | 1.714 | 1.872 | 1.790 | 1.912 | 1.489 | 1.880 |
| 1/200 | 2.422 | 1.540 | 1.773 | 1.658 | 1.724 | 1.471 | 2.006 |
| 1/400 | 2.456 | 1.266 | 1.783 | 1.907 | 1.312 | 1.223 | 1.919 |
| 1/800 | 2.196 | 0.928 | 1.825 | 2.059 | 1.478 | 1.136 | 2.197 |
| 1/1600 | 1.659 | 0.648 | 1.749 | 2.062 | 1.233 | 1.157 | 1.672 |
| Titer | >1600 | 800 | >1600 | >1600 | >1600 | >1600 | >1600 |

TABLE 5
Protocol 1: mock immunization (PBS), ELISA results
Mouse

| | | | | | | | | | | |
|---|---|---|---|---|---|---|---|---|---|---|
| Dilution | 1 | 2 | 3 | 4 | 5 | 6 | 7 | 8 | 9 | 10 |

1^{st} immunization (day 0)

| | | | | | | | | | | |
|---|---|---|---|---|---|---|---|---|---|---|
| 1/50 | 0.135 | 0.166 | 0.196 | 0.169 | 0.207 | 0.187 | 0.207 | 0.142 | 0.131 | 0.220 |
| 1/100 | 0.152 | 0.119 | 0.134 | 0.128 | 0.124 | 0.116 | 0.146 | 0.193 | 0.116 | 0.105 |
| 1/200 | 0.116 | 0.112 | 0.121 | 0.115 | 0.112 | 0.108 | 0.108 | 0.127 | 0.103 | 0.100 |
| 1/400 | 0.111 | 0.107 | 0.141 | 0.127 | 0.108 | 0.104 | 0.113 | 0.092 | 0.102 | 0.100 |
| 1/800 | 0.106 | 0.111 | 0.116 | 0.123 | 0.104 | 0.105 | 0.111 | 0.084 | 0.107 | 0.099 |
| 1/1600 | 0.129 | 0.115 | 0.109 | 0.115 | 0.102 | 0.102 | 0.105 | 0.068 | 0.106 | 0.096 |
| Titer | 0 | 0 | 0 | 0 | 0 | 0 | 0 | 0 | 0 | 0 |

2^{nd} immunization (day 13)

| | | | | | | | | | | |
|---|---|---|---|---|---|---|---|---|---|---|
| 1/50 | 0.050 | 0.056 | 0.048 | 0.056 | 0.050 | 0.050 | 0.048 | 0.050 | 0.269 | 0.374 |
| 1/100 | 0.047 | 0.046 | 0.480 | 0.046 | 0.048 | 0.048 | 0.054 | 0.048 | 0.133 | 0.185 |
| 1/200 | 0.122 | 0.114 | 0.125 | 0.121 | 0.117 | 0.115 | 0.121 | 0.116 | 0.117 | 0.184 |
| 1/400 | 0.108 | 0.116 | 0.137 | 0.118 | 0.109 | 0.105 | 0.113 | 0.108 | 0.108 | 0.144 |
| 1/800 | 0.109 | 0.114 | 0.139 | 0.111 | 0.106 | 0.107 | 0.108 | 0.105 | 0.094 | 0.135 |
| 1/1600 | 0.109 | 0.110 | 0.117 | 0.105 | 0.104 | 0.109 | 0.104 | 0.109 | 0.115 | 0.119 |
| Titer | 0 | 0 | 0 | 0 | 0 | 0 | 0 | 0 | 0 | 0 |

3^{d} immunization (day 35)

| | | | | | | | | | | |
|---|---|---|---|---|---|---|---|---|---|---|
| 1/50 | 0.224 | 0.238 | 0.183 | 0.205 | 0.266 | 0.257 | 0.664 | 0.154 | 0.202 | 0.19 [symbol] |
| 1/100 | 0.140 | 0.139 | 0.129 | 0.141 | 0.153 | 0.163 | 0.291 | 0.113 | 0.164 | 0.15 [symbol] |
| 1/200 | 0.117 | 0.126 | 0.104 | 0.112 | 0.140 | 0.148 | 0.212 | 0.106 | 0.134 | 0.11 [symbol] |
| 1/400 | 0.125 | 0.166 | 0.102 | 0.098 | 0.113 | 0.118 | 0.154 | 0.089 | 0.120 | 0.11 [symbol] |
| 1/800 | 0.116 | 0.117 | 0.102 | 0.102 | 0.099 | 0.100 | 0.129 | 0.090 | 0.113 | 0.10 [symbol] |
| 1/1600 | 0.113 | 0.118 | 0.098 | 0.111 | 0.104 | 0.069 | 0.046 | 0.116 | 0.139 | 0.10 [symbol] |
| Titer | 0 | 0 | 0 | 0 | 0 | 0 | 0 | 0 | 0 | 0 |

Sacrifice (day 53)

| | | | | | | | | | | |
|---|---|---|---|---|---|---|---|---|---|---|
| 1/50 | 0.233 | 0.169 | 0.183 | 0.266 | 0.409 | 0.224 | 0.238 | 0.193 | 0.212 | 0.22 |
| 1/100 | 0.144 | 0.126 | 0.132 | 0.174 | 0.254 | 0.157 | 0.199 | 0.160 | 0.209 | 0.14 |
| 1/200 | 0.128 | 0.110 | 0.117 | 0.134 | 0.196 | 0.131 | 0.180 | 0.135 | 0.186 | 0.13 |
| 1/400 | 0.126 | 0.102 | 0.103 | 0.117 | 0.153 | 0.119 | 0.124 | 0.129 | 0.116 | 0.11 |
| 1/800 | 0.122 | 0.103 | 0.103 | 0.111 | 0.134 | 0.113 | 0.114 | 0.125 | 0.113 | 0.11 |
| 1/1600 | 0.119 | 0.105 | 0.106 | 0.109 | 0.120 | 0.109 | 0.102 | 0.117 | 0.095 | 0.11 |
| Titer | 0 | 0 | 0 | 0 | 0 | 0 | 0 | 0 | 0 | 0 |

[symbol]

indicates data missing or illegible when filed

TABLE 6
Protocol 1: ELISA controls

| Dilution | | Control 1 | Control 2 | Control 3 | Control 4 | Control 5 | Control 6 |
|---|---|---|---|---|---|---|---|
| | Ag | 0 | 0 | ARFP | ARFP | ARFP | ARFP |
| | Ab 1 | 0 | Serum | Serum | 0 | Anti-ARFP | Serum |
| | | | [N^{o} 31, | [N^{o} 31, | | rabbit | [N^{o} 31, |
| | | | TP4] | TP4] | | polyclonal | TP4] |
| | Ab 2 | 0 | AP | 0 | AP | AP | AP |
| | | | conjugated | | conjugated | conjugated | conjugated |
| | | | goat anti- | | goat anti- | goat anti- | mouse |
| | | | mouse Ig | | mouse Ig | rabbit IgG | anti-human |
| | | | [G, A, M] | | [G, A, M] | | IgG |
| 1/50 | | 0.131 | 0.290* | 0.136 | 0.194 | 1.759 | 0.157 |
| 1/100 | | 0.125 | 0.125 | 0.125 | 0.127 | 1.333 | 0.118 |
| 1/200 | | 0.109 | 0.147 | 0.121 | 0.106 | 1.494 | 0.104 |
| 1/400 | | 0.102 | 0.128 | 0.116 | 0.110 | 1.410 | 0.113 |
| 1/800 | | 0.113 | 0.131 | 0.110 | 0.109 | 1.127 | 0.102 |
| 1/1600 | | 0.108 | 0.131 | 0.117 | 0.109 | 1.974 | 0.144 |
| Titer | | 0 | 0 | 0 | 0 | >1600 | 0 |

*OD 410 value used for determining the detection threshold.

TABLE 7
Protocol 2: ARFP immunization, ELISA results

| | Mouse | | | | | | | |
|---|---|---|---|---|---|---|---|---|
| Dilution | 1 | 2 | 3 | 4 | 5 | 6 | 7 | 8 |

1^{st} immunization (day 0)

| | | | | | | | | |
|---|---|---|---|---|---|---|---|---|
| 1/50 | 0.166 | 0.236 | 0.243 | 0.247 | 0.208 | 0.206 | 0.205 | 0.217 |
| 1/100 | 0.115 | 0.161 | 0.161 | 0.145 | 0.134 | 0.132 | 0.142 | 0.127 |
| 1/200 | 0.123 | 0.150 | 0.128 | 0.136 | 0.114 | 0.111 | 0.121 | 0.113 |
| 1/400 | 0.132 | 0.164 | 0.114 | 0.118 | 0.116 | 0.121 | 0.133 | 0.115 |
| 1/800 | 0.119 | 0.138 | 0.113 | 0.118 | 0.115 | 0.117 | 0.128 | 0.115 |
| 1/1600 | 0.138 | 0.122 | 0.109 | 0.110 | 0.109 | 0.116 | 0.124 | 0.108 |
| Titer | 0 | 0 | 0 | 0 | 0 | 0 | 0 | 0 |

2^{nd} immunization (day 14)

| | | | | | | | | |
|---|---|---|---|---|---|---|---|---|
| 1/50 | 0.370 | 0.625 | 0.303 | 0.339 | 0.421 | 3.852 | 0.552 | 0.270 |
| 1/100 | 0.187 | 0.306 | 0.155 | 0.164 | 0.156 | 1.842 | 0.204 | 0.153 |
| 1/200 | 0.168 | 0.183 | 0.128 | 0.126 | 0.133 | 1.337 | 0.149 | 0.124 |
| 1/400 | 0.173 | 0.152 | 0.136 | 0.129 | 0.122 | 0.777 | 0.133 | 0.123 |
| 1/800 | 0.156 | 0.130 | 0.123 | 0.121 | 0.121 | 0.539 | 0.125 | 0.124 |
| 1/1600 | 0.137 | 0.126 | 0.118 | 0.122 | 0.119 | 0.546 | 0.125 | 0.146 |
| Titer | 0 | 0 | 0 | 0 | 0 | 200 | 0 | 0 |

3^{d} immunization (day 29)

| | | | | | | | | |
|---|---|---|---|---|---|---|---|---|
| 1/50 | 3.831 | 3.849 | 3.373 | 3.573 | 2.987 | 2.874 | 2.626 | 3.470 |
| 1/100 | 3.525 | 3.898 | 2.898 | 1.264 | 0.982 | 1.468 | 1.197 | 2.613 |
| 1/200 | 2.387 | 3.359 | 1.364 | 1.013 | 0.684 | 0.906 | 0.657 | 3.189 |
| 1/400 | 1.910 | 2.646 | 1.019 | 0.830 | 0.473 | 0.493 | 0.397 | 3.666 |
| 1/800 | 1.316 | 1.965 | 0.647 | 0.556 | 0.366 | 0.536 | 0.266 | 3.106 |
| 1/1600 | 0.770 | 1.960 | 0.558 | 0.428 | 0.284 | 0.513 | 0.298 | 3.423 |
| Titer | 800 | >1600 | 400 | 400 | 100 | 200 | 100 | >1600 |

Sacrifice (day 52)

| | | | | | | | | |
|---|---|---|---|---|---|---|---|---|
| 1/50 | 3.593 | 3.623 | 2.931 | 3.934 | 3.865 | >4.000 | 3.796 | 3.843 |
| 1/100 | 3.071 | 3.581 | 2.555 | 3.459 | >4.000 | >4.000 | 3.873 | 3.848 |
| 1/200 | 1.902 | 1.744 | 1.431 | 2.418 | 2.646 | >4.000 | 3.807 | 3.818 |
| 1/400 | 1.187 | 1.526 | 0.987 | 1.257 | 2.378 | >4.000 | 3.802 | 3.891 |
| 1/800 | 1.110 | 1.341 | 0.818 | 1.093 | 2.236 | >4.000 | 3.867 | 3.833 |
| 1/1600 | 1.589 | 1.086 | 1.084 | 0.771 | 1.427 | >4.000 | 3.813 | 3.843 |
| Titer | >1600 | >1600 | >1600 | 800 | >1600 | >1600 | >1600 | >1600 |

| | Mouse | | | | | | |
|---|---|---|---|---|---|---|---|
| Dilution | 9 | 10 | 21 | 22 | 23 | 24 | 25 |
| 1^{st} immunization (day 0) | | | | | | | |
| 1/50 | 0.211 | 0.209 | 0.199 | 0.218 | 0.323 | 0.278 | 0.229 |
| 1/100 | 0.128 | 0.115 | 0.174 | 0.208 | 0.186 | 0.171 | 0.152 |
| 1/200 | 0.113 | 0.104 | 0.117 | 0.136 | 0.164 | 0.144 | 0.140 |
| 1/400 | 0.104 | 0.110 | 0.120 | 0.128 | 0.191 | 0.138 | 0.126 |
| 1/800 | 0.108 | 0.103 | 0.105 | 0.123 | 0.134 | 0.137 | 0.114 |
| 1/1600 | 0.106 | 0.107 | 0.105 | 0.119 | 0.129 | 0.127 | 0.114 |
| Titer | 0 | 0 | 0 | 0 | 0 | 0 | 0 |
| 2^{nd} immunization (day 14) | | | | | | | |
| 1/50 | 2.758 | 0.505 | >4.000 | 0.189 | 3.567 | >4.000 | >4.000 |
| 1/100 | 0.560 | 0.191 | 3.323 | 0.141 | 3.184 | >4.000 | 1.876 |
| 1/200 | 0.295 | 0.144 | 2.201 | 0.112 | 3.227 | >4.000 | 1.851 |
| 1/400 | 0.204 | 0.131 | 1.212 | 0.111 | 1.433 | 2.473 | 0.736 |
| 1/800 | 0.173 | 0.122 | 0.622 | 0.113 | 0.790 | 2.199 | 0.427 |
| 1/1600 | 0.154 | 0.115 | 0.337 | 0.113 | 0.462 | 1.296 | 0.271 |
| Titer | 50 | 0 | 400 | 0 | 400 | >1600 | 200 |
| 3^{d} immunization (day 29) | | | | | | | |
| 1/50 | 3.517 | 1.225 | 3.635 | 0.801 | 3.936 | 3.926 | >4.000 |
| 1/100 | 1.648 | 0.560 | >4.000 | 0.304 | 3.959 | 3.978 | >4.000 |
| 1/200 | 1.518 | 0.303 | 3.924 | 0.240 | 3.936 | 3.853 | >4.000 |
| 1/400 | 1.315 | 0.291 | 3.086 | 0.174 | 3.874 | 3.765 | >4.000 |
| 1/800 | 1.194 | 0.218 | >4.000 | 0.188 | 3.874 | 3.903 | 3.429 |
| 1/1600 | 1.055 | 0.187 | 3.573 | 0.266 | 3.907 | 3.939 | 3.210 |
| Titer | >1600 | 50 | >1600 | 0 | >1600 | >1600 | >1600 |
| Sacrifice (day 52) | | | | | | | |
| 1/50 | 3.456 | 3.855 | 3.717 | 3.534 | 3.636 | 3.780 | 3.783 |
| 1/100 | 3.622 | 3.966 | 1.524 | 2.193 | 3.518 | 2.628 | 3.952 |
| 1/200 | 3.641 | 3.184 | 3.740 | 1.047 | 2.844 | 2.829 | 3.142 |
| 1/400 | 3.742 | 3.885 | 3.660 | 1.001 | 3.548 | 3.151 | 3.752 |
| 1/800 | 3.665 | 3.171 | 3.717 | 0.856 | 3.406 | 3.707 | 3.620 |
| 1/1600 | 3.612 | 3.055 | 3.832 | 0.510 | 3.562 | 3.780 | 3.976 |
| Titer | >1600 | >1600 | >1600 | 800 | >1600 | >1600 | >1600 |

TABLE 8
Protocol 2: mock immunization (PBS), ELISA results

| | Mouse | | | | | | | | | | | | | |
|---|---|---|---|---|---|---|---|---|---|---|---|---|---|---|
| Dilution | 11 | 12 | 13 | 14 | 15 | 16 | 17 | 18 | 19 | 20 | 26 | 27 | 28 | 30 |

1^{st} immunization (day 0)

| | | | | | | | | | | | | | | |
|---|---|---|---|---|---|---|---|---|---|---|---|---|---|---|
| 1/50 | 0.283 | 0.247 | 0.680 | 0.158 | 0.144 | 0.164 | 0.141 | 0.160 | 0.187 | 0.297 | 0.441 | 0.320 | 0.325 | 0.353 |
| 1/100 | 0.162 | 0.130 | 0.310 | 0.128 | 0.115 | 0.116 | 0.114 | 0.117 | 0.128 | 0.213 | 0.258 | 0.184 | 0.197 | 0.227 |
| 1/200 | 0.132 | 0.120 | 0.211 | 0.126 | 0.105 | 0.107 | 0.102 | 0.105 | 0.122 | 0.197 | 0.188 | 0.139 | 0.161 | 0.146 |
| 1/400 | 0.128 | 0.119 | 0.173 | 0.121 | 0.111 | 0.107 | 0.109 | 0.113 | 0.114 | 0.161 | 0.151 | 0.131 | 0.136 | 0.135 |
| 1/800 | 0.116 | 0.119 | 0.168 | 0.116 | 0.107 | 0.112 | 0.106 | 0.106 | 0.110 | 0.164 | 0.134 | 0.128 | 0.134 | 0.123 |
| 1/1600 | 0.116 | 0.121 | 0.153 | 0.122 | 0.109 | 0.110 | 0.107 | 0.107 | 0.109 | 0.131 | 0.127 | 0.118 | 0.122 | 0.121 |
| Titer | 0 | 0 | 0 | 0 | 0 | 0 | 0 | 0 | 0 | 0 | 0 | 0 | 0 | 0 |

2^{nd} immunization (day 14)

| | | | | | | | | | | | | | | |
|---|---|---|---|---|---|---|---|---|---|---|---|---|---|---|
| 1/50 | 0.108 | 0.442 | 0.288 | 0.212 | 0.229 | 0.249 | 0.670 | 0.228 | 0.230 | 0.245 | 0.281 | 0.216 | 0.261 | nd |
| 1/100 | 0.154 | 0.302 | 0.177 | 0.171 | 0.162 | 0.174 | 0.487 | 0.162 | 0.180 | 0.168 | 0.193 | 0.146 | 0.179 | nd |
| 1/200 | 0.135 | 0.215 | 0.151 | 0.178 | 0.146 | 0.130 | 0.321 | 0.136 | 0.144 | 0.138 | 0.146 | 0.113 | 0.146 | nd |
| 1/400 | 0.116 | 0.170 | 0.143 | 0.151 | 0.144 | 0.121 | 0.248 | 0.127 | 0.142 | 0.121 | 0.133 | 0.115 | 0.133 | nd |
| 1/800 | 0.114 | 0.160 | 0.169 | 0.150 | 0.118 | 0.130 | 0.184 | 0.126 | 0.130 | 0.136 | 0.133 | 0.113 | 0.122 | nd |
| 1/1600 | 0.113 | 0.150 | 0.142 | 0.143 | 0.116 | 0.123 | 0.159 | 0.122 | 0.129 | 0.118 | 0.126 | 0.112 | 0.114 | nd |
| Titer | 0 | 0 | 0 | 0 | 0 | 0 | 0 | 0 | 0 | 0 | 0 | 0 | 0 | nd |

3^{d} immunization (day 29)

| | | | | | | | | | | | | | | |
|---|---|---|---|---|---|---|---|---|---|---|---|---|---|---|
| 1/50 | 0.307 | 0.246 | 0.338 | 0.246 | 0.253 | 0.327 | 0.754 | 0.356 | 0.444 | 0.510 | 0.416 | 0.324 | 0.354 | 0.381 |
| 1/100 | 0.190 | 0.148 | 0.161 | 0.156 | 0.160 | 0.325 | 0.269 | 0.217 | 0.270 | 0.181 | 0.178 | 0.175 | 0.204 | 0.224 |
| 1/200 | 0.153 | 0.127 | 0.126 | 0.143 | 0.123 | 0.196 | 0.191 | 0.166 | 0.184 | 0.154 | 0.154 | 0.140 | 0.167 | 0.157 |
| 1/400 | 0.128 | 0.114 | 0.121 | 0.126 | 0.113 | 0.151 | 0.138 | 0.134 | 0.540 | 0.133 | 0.144 | 0.130 | 0.143 | 0.132 |
| 1/800 | 0.127 | 0.114 | 0.126 | 0.123 | 0.130 | 0.129 | 0.119 | 0.126 | 0.139 | 0.130 | 0.144 | 0.124 | 0.128 | 0.126 |
| 1/1600 | 0.123 | 0.117 | 0.13 | 0.124 | 0.122 | 0.116 | 0.112 | 0.119 | 0.119 | 0.141 | 0.137 | 0.129 | 0.128 | 0.129 |
| Titer | 0 | 0 | 0 | 0 | 0 | 0 | 0 | 0 | 0 | 0 | 0 | 0 | 0 | 0 |

Sacrifice (day 52)

| | | | | | | | | | | | | | | |
|---|---|---|---|---|---|---|---|---|---|---|---|---|---|---|
| 1/50 | 0.350 | 0.516 | 0.474 | 0.288 | 0.376 | 0.231 | 0.500 | 0.279 | 0.313 | 0.247 | 0.203 | 0.201 | 0.165 | 0.226 |
| 1/100 | 0.173 | 0.208 | 0.238 | 0.224 | 0.215 | 0.194 | 0.286 | 0.161 | 0.175 | 0.182 | 0.146 | 0.155 | 0.141 | 0.148 |
| 1/200 | 0.133 | 0.170 | 0.172 | 0.156 | 0.151 | 0.149 | 0.185 | 0.135 | 0.143 | 0.146 | 0.123 | 0.136 | 0.117 | 0.128 |
| 1/400 | 0.149 | 0.146 | 0.133 | 0.163 | 0.177 | 0.134 | 0.142 | 0.120 | 0.135 | 0.141 | 0.120 | 0.120 | 0.119 | 0.119 |
| 1/800 | 0.118 | 0.125 | 0.123 | 0.159 | 0.134 | 0.107 | 0.125 | 0.114 | 0.120 | 0.127 | 0.114 | 0.115 | 0.107 | 0.113 |
| 1/1600 | 0.117 | 0.120 | 0.113 | 0.145 | 0.136 | 0.123 | 0.125 | 0.134 | 0.126 | 0.127 | 0.118 | 0.116 | 0.123 | 0.119 |
| Titer | 0 | 0 | 0 | 0 | 0 | 0 | 0 | 0 | 0 | 0 | 0 | 0 | 0 | 0 |

TABLE 9
Protocol 2: ELISA controls

| Dilution | | Control 3 | Control 4 | Control 5 | Control 6 |
|---|---|---|---|---|---|
| | Ag | ARFP | ARFP | ARFP | ARFP |
| | Ab 1 | Serum | 0 | Anti-ARFP | Serum |
| | | [N^{o} 30, | | rabbit | [N^{o} 30, |
| | | TP3] | | polyclonal | TP3] |
| | Ab 2 | 0 | AP | AP | AP |
| | | | conjugated | conjugated | conjugated |
| | | | goat anti- | goat anti- | mouse |
| | | | mouse Ig | rabbit IgG | anti-human |
| | | | [G, A, M] | | IgG |
| 1/50 | | 0.268* | 0.131 | 3.926 | 0.174 |
| 1/100 | | 0.155 | 0.129 | 3.918 | 0.158 |
| 1/200 | | 0.135 | 0.108 | 3.916 | 0.156 |
| 1/400 | | 0.123 | 0.111 | 3.748 | 0.155 |
| 1/800 | | 0.122 | 0.112 | 3.733 | 0.149 |
| 1/1600 | | 0.135 | 0.111 | 3.017 | 0.181 |
| Titer | | 0 | 0 | >1600 | 0 |

*OD 410 value used for determining the detection threshold.

TABLE 10

Correspondence of SEQ ID NOs: with sequences
described herein

| SEQ | | |
|---|---|---|
| ID | | |
| NO: | Sequence | Description |
| 1 | GCC AGC CCC | primer |
| | CTG ATG G | |
| 2 | GCC TCG TAC | primer |
| | ACA ATA CTC | |
| | G | |
| 3 | GAC CGT CGA | primer |
| | CCA TGA GCA | |
| | CGA ATC CTA | |
| | AAC CTC AGA | |
| | GGA AGA CCC | |
| | CAA ACG TAA | |
| 4 | AAG GGT ACC | primer |
| | CGG GCT GAG | |
| | CCC AGG TCC | |
| | TGC CCT CGG | |
| | G | |
| 5 | TCA AGT CGA | primer |
| | CCC AAA CGT | |
| | AAC ACC AAC | |
| | CG | |
| 6 | GGA AAC AGC | primer |
| | TAT GAC CAT | |
| | GAT TAC GCC | |
| | AAG C | |
| 7 | See below | DNA encoding F protein |
| | | derived from Accession |
| | | M62321 |
| 8 | See below | F protein polypeptide |
| | | derived from Accession |
| | | M62321 |
| 9 | See below | Δ11 truncated F protein |
| | | polypeptide; truncation of |
| | | SEQ ID NO: 8 |
| 10 | See below | DNA encoding Fmut8 protein |
| | | identified in studies described |
| | | herein |
| 11 | See FIG. 1C | Fmut8 protein polypeptide |
| | and below | identified in studies described |
| | herein; encoded by SEQ ID NO: 10 | |
| 12 | See below | FmutBΔ11 polypeptide identified |
| | | in studies described herein; |
| | | truncation of SEQ ID NO: 11 |
| 13 | KLVALGINAV | HCV-1 N53 1406-1415 peptide |
| 14 | VRSLVEFTCCRAG | F protein peptide F29-43 |
| | AL | |
| 15 | SLVEFTCCRA | F protein peptide F31-40; |
| | | epitope located within F protein |
| | | peptide F29-43 for HCV subtype |
| | | 1a [M62321]* (see Table 3) |
| 16 | SLAEYTCCRA | epitope located within F protein |
| | | peptide F29-43 for HCV subtype |
| | | 2a [D00944]* (see Table 3) |
| 17 | SLVEYTCCRA | epitope located within F protein |
| | | peptide F29-43 for HCV subtype |
| | | 3a [D17763]* (see Table 3) |
| 18 | SLAEFTCCRA | epitope located within F protein |
| | | peptide F29-43 for HCV subtype |
| | | 4a [Y11604]* (see Table 3) |
| 19 | SLVEFTCCRA | epitope located within F protein |
| | | peptide F29-43 for HCV subtype |
| | | 5a [Y13184]* (see Table 3) |
| 20 | SLAEFTCCRA | epitope located within F protein |
| | | peptide F29-43 for HCV subtype |
| | | 6a [Y12083]* (see Table 3) |
| 21 | CCRAGALDWVCAR | F protein peptide F37-51 |
| | RE | |
| 22 | CRAGALDWV | F protein peptide F38-46; |
| | | epitope located within F protein |
| | | peptide F37-51 for HCV subtype |
| | | 1a [M62321]* (see Table 3) |
| 23 | CCRAGALDWV | F protein peptide F37-46 |
| 24 | CRAGAPGWV | epitope located within F protein |
| | | peptide F37-51 for HCV subtype |
| | | 2a [D00944]* (see Table 3) |
| 25 | CRAGAHDWV | epitope located within F protein |
| | | peptide F37-51 for HCV subtype |
| | | 3a [D17763]* (see Table 3) |
| 26 | CRAGAPDWV | epitope located within F protein |
| | | peptide F37-51 for HCV subtype |
| | | 4a [Y11604]* (see Table 3) |
| 27 | CRAGALNWV | epitope located within F protein |
| | | peptide F37-51 for HCV subtype |
| | | 5a [Y13184]* (see Table 3) |
| 28 | CRARAPGWV | epitope located within F protein |
| | | peptide F37-51 for HCV subtype |
| | | 6a [Y12083]* (see Table 3) |
| 29 | PVALGLAGAPQTP | F protein peptide F101-115 |
| | GV | |
| 30 | ALGLAGAPQT | F protein peptide F103-112; |
| | | epitope located within F protein |
| | | peptide F101-115 for HCV subtype |
| | | 1a [M62321]* (see Table 3) |
| 31 | GLAGAPQTP | F protein peptide F105-113 |
| 32 | AGAPQTPGV | F protein peptide F107-115 |
| 33 | LAGAPQTPGV | F protein peptide F106-115 |
| 34 | VPVPLGAPMT | epitope located within F protein |
| | | peptide F101-115 for HCV subtype |
| | | 2a [D00944]* (see Table 3) |
| 35 | APVHPGAQMT | epitope located within F protein |
| | | peptide F101-115 for HCV subtype |
| | | 3a [D17763]* (see Table 3) |
| 36 | ALDRLGAQMI | epitope located within F protein |
| | | peptide F101-115 for HCV subtype |
| | | 4a [Y11604]* (see Table 3) |
| 37 | ALGLIGAPMT | epitope located within F protein |
| | | peptide F101-115 for HCV subtype |
| | | 5a [Y13184]* (see Table 3) |
| 38 | APGHTGAPMT | epitope located within F protein |
| | | peptide F101-115 for HCV subtype |
| | | 6a [Y12083]* (see Table 3) |
| 39 | MSTNPKPQRK | peptide of mass spectrometry |
| | | analysis (See FIG. 1C) |
| 40 | VAVRSLVEFTCCR | peptide of mass spectrometry |
| | | analysis (See FIG. 1C) |
| 41 | AGALDWVCAR | peptide of mass spectrometry |
| | | analysis (See FIG. 1C) |
| 42 | RGRLPSGRNLEVD | peptide of mass spectrometry |
| | VSLSPR | analysis (See FIG. 1C) |
| 43 | GRLPSGRNLE | peptide of mass spectrometry |
| | | analysis (See FIG. 1C) |
| 44 | VDVSLSPRHVGPR | peptide of mass spectrometry |
| | | analysis (See FIG. 1C) |
| 45 | AGPGLSPGTLGPS | peptide of mass spectroinetry |
| | MAMR | analysis (See FIG. 1C) |
| 46 | DGSCLPVALGLVG | peptide of mass spectrometry |
| | APQTPGVGR | analysis (See FIG. 1C) |
| 47 | AIWVRSSIPSR | peptide of mass spectrometry |
| | | analysis (See FIG. 1C) |
| 48 | AASPTSWGTYR | peptide of mass spectrometry |
| | | analysis (See FIG. 1C) |
| 49 | AASPTSWGTYRSS | peptide of mass spectrometry |
| | APPLEALPGPWR | analysis (See FIG. 1C) |
| 50 | MASGFWR | peptide of mass spectrometry |
| | | analysis (See FIG. 1C) |
| 51 | VRSLVEFTCCRA | F protein peptide F29-40; HCV |
| | | subtype 1a [M62321]* |
| 52 | VRSLVEFTCCRAG | F protein peptide F29-51; HCV |
| | ALDWVCARRE | subtype 1a [M62321]* |
| 53 | SLVEFTCCRAGAL | F protein peptide F31-43; HCV |
| | | subtype 1a [M62321]* |
| 54 | SLVEFTCCRAGAL | F protein peptide F31-51; HCV |
| | DWVCARRE | subtype 1a [M62321]* |
| 55 | ARSLAEYTCCRA | F protein peptide F29-40; HCV |
| | | subtype 2a [D00944]* |
| 56 | ARSLAEYTCCRAGA | F protein peptide F29-43; HCV |
| | P | subtype 2a [D00944]* |
| 57 | ARSLAEYTCCRAG | F protein peptide F29-51; HCV |
| | APGWVCARQG | subtype 2a [D00944]* |
| 58 | SLAEYTCCRAGAP | F protein peptide F31-43; HCV |
| | | subtype 2a [D00944]* |
| 59 | SLAEYTCCRAGAP | F protein peptide F31-51; HCV |
| | GWVCARQG | subtype 2a [D00944]* |
| 60 | CCRAGAPGWVCAR | F protein peptide F37-51; HCV |
| | QG | subtype 2a [D00944]* |
| 61 | PVALGLAGAPQTP | F protein peptide F101-115; HCV |
| | GV | subtype 2a [D00944]* |
| 62 | AGAPQTPGV | F protein peptide F107-115; HCV |
| | | subtype 2a [D00944]* |
| 63 | DRSLVEYTCCRA | F protein peptide F29-40; HCV |
| | | subtype 3a [D17763]* |
| 64 | DRSLVEYTCCRAG | F protein peptide F29-43; HCV |
| | AH | subtype 3a [D17763]* |
| 65 | DRSLVEYTCCRAG | F protein peptide F29-51; HCV |
| | AHDWVCARRV | subtype 3a [D17763]* |
| 66 | SLVEYTCCRAGAH | F protein peptide F31-43; HCV |
| | | subtype 3a [D17763]* |
| 67 | SLVEYTCCRAGAH | F protein peptide F31-51; HCV |
| | DWVCARRV | subtype 3a [D17763]* |
| 68 | CCRAGAHDWVCAR | F protein peptide F37-51; HCV |
| | RV | subtype 3a [D17763]* |
| 69 | HAAPVHPGAQMTP | F protein peptide F101-115; HCV |
| | GG | subtype 3a [D17763]* |
| 70 | PGAQMTPGG | F protein peptide F107-115; HCV |
| | | subtype 3a [D17763]* |
| 71 | ARSLAEFTCCRA | F protein peptide F29-40; HCV |
| | | subtype 4a [Y11604]* |
| 72 | ARSLAEFTCCRAG | F protein peptide F29-43; HCV |
| | AP | subtype 4a [Y11604]* |
| 73 | ARSLAEFTCCRAG | F protein peptide F29-51; HCV |
| | APDWVSARLG | subtype 4a [Y11604]* |
| 74 | SLAEFTCCRAGAP | F protein peptide F31-43; HCV |
| | | subtype 4a [Y11604]* |
| 75 | SLAEFTCCRAGAP | F protein peptide F31-51; HCV |
| | DWVSARLG | subtype 4a [Y11604]* |
| 76 | CCRAGAPDWVSAR | F protein peptide F37-51; HCV |
| | LG | subtype 4a [Y11604]* |
| 77 | PVALDRLGAQMIP | F protein peptide F101-115; HCV |
| | AG | subtype 4a [Y11604]* |
| 78 | LGAQMIPAG | F protein peptide F107-115; HCV |
| | | subtype 4a [Y11604]* |
| 79 | VRSLVEFTCCRA | F protein peptide F29-40; HCV |
| | | subtype 5a [Y13184]* |
| 80 | VRSLVEFTCCRAG | F protein peptide F29-43; HCV |
| | AL | subtype 5a [Y13184]* |
| 81 | VRSLVEFTCCRAG | F protein peptide F29-51; HCV |
| | ALNWVSARLG | subtype 5a [Y13184]* |
| 82 | SLVEFTCCRAGAL | F protein peptide F31-43; HCV |
| | | subtype 5a [Y13184]* |
| 83 | SLVEFTCCRAGAL | F protein peptide F31-51; HCV |
| | NWVSARLG | subtype 5a [Y13184]* |
| 84 | CCRAGALNWVSAR | F protein peptide F37-51; HCV |
| | LG | subtype 5a [Y13184]* |
| 85 | PEALGLIGAPMTP | F protein peptide F101-115; HCV |
| | GG | subtype 5a [Y13184]* |
| 86 | IGAPMTPGG | F protein peptide F107-115; HCV |
| | | subtype 5a [Y13184]* |
| 87 | VRSLAEFTCCRA | F protein peptide F29-40; HCV |
| | | subtype 6a [Y12083]* |
| 88 | VRSLAEFTCCRAR | F protein peptide F29-43; HCV |
| | AP | subtYpe 6a [Y12083]* |
| 89 | VRSLAEFTCCRAR | F protein peptide F29-51; HCV |
| | APGWVCARRG | subtype 6a [Y12083]* |
| 90 | SLAEFTCCRARAP | F protein peptide F31-43; HCV |
| | | subtype 6a [Y12083]* |
| 91 | SLAEFTCCRARAP | F protein peptide F31-51; HCV |
| | GWVCARRG | subtype 6a [Y12083]* |
| 92 | CCRARAPGWVCAR | F protein peptide F37-51; HCV |
| | RG | subtype 6a [Y12083]* |
| 93 | PAAPGHTGAPMTP | F protein peptide F101-115; HCV |
| | GV | subtype 6a [Y12083]* |
| 94 | TGAPMTPGV | F protein peptide F107-115; HCV |
| | | subtype 6a [Y12083]* |
| 95 | VRSLVEFTCCRA | F protein peptide F29-40; Fmut 8 |
| 96 | VRSLVEFTCCRAG | F protein peptide F29-43; Fmut 8 |
| | AL | |
| 97 | VRSLVEFTCCRAG | F protein peptide F29-51; Fmut 8 |
| | ALDWVCARRG | |
| 98 | SLVEFTCCRA | F protein peptide F31-40; Fmut 8 |
| 99 | SLVEFTCCRAGAL | F protein peptide F31-43; Fmut 8 |
| 100 | SLVEFTCCRAGAL | F protein peptide F31-51; Fmut 8 |
| | DWVCARRG | |
| 101 | CCRAGALDWVCAR | F protein peptide F37-51; Fmut 8 |
| | RG | |
| 102 | PVALGLVGAPQTP | F protein peptide F101-115; Fmut |
| | GV | 8 |
| 103 | VGAPQTPGV | F protein peptide F107-115; Fmut |
| | | 8 |
| 104 | GTCAGATCGTTGG | Nucleotide sequences of ARFP- |
| | TGGAGTTTACTTG | derived F29-51; HCV subtype 1a |
| | TTGCCGCGCAGGG | |
| | GCCCTAGATTGGG | |
| | TGTGCGCGCGACG | |
| | AGAA | |
| 105 | GCCAGATCGTTGG | Nucleotide sequences of ARFP- |
| | CGGAGTATACTTG | derived F29-51; HCV subtype 2a |
| | TTGCCGCGCAGGG | |
| | GCCCCAGGTTGGG | |
| | TGTGCGCGCGACA | |
| | AGGA | |
| 106 | GACAGATCGTTGG | Nucleotide sequences of ARFP- |
| | TGGAGTATACGTG | derived F29-51; HCV subtype 3a |
| | TTGCCGCGCAGGG | |
| | GCCCACGATTGGG | |
| | TGTGCGCGCGACG | |
| | CGTA | |
| 107 | GCCAGATCGTTGG | Nucleotide sequences of ARFP- |
| | CGGAGTTTACTTG | derived F29-51; HCV subtype 4a |
| | TTGCCGCGCAGGG | |
| | GCCCCAGATTGGG | |
| | TGTGCGCGCGACT | |
| | CGGA | |
| 108 | GTCAGATCGTTGG | Nucleotide sequences of ARFP- |
| | TGGAGTTTACTTG | derived F29-51; HCV subtype 5a |
| | TTGCCGCGCAGGG | |
| | GCCCTAAATTGGG | |
| | TGTGCGCGCGACT | |
| | CGGA | |
| 109 | GTCAGATCGTTGG | Nucleotide sequences of ARFP- |
| | CGGAGTTTACTTG | derived F29-51; HCV subtype 6a |
| | TTGCCGCGCAAGG | |
| | GCCCCCGGTTGGG | |
| | TGTGCGCGCGACG | |
| | AGGA | |
| 110 | CCCGTGGCTCTCG | Nucleotide sequences of ARFP- |
| | GCCTAGCTGGGGC | derived F101-115; HCV subtype 1a |
| | CCCACAGACCCCC | |
| | GGCGTA | |
| 111 | CCCGAGGTTCCCG | Nucleotide sequences of ARFP- |
| | TCCCTCTTGGGGC | derived F101-115; HCV subtype 2a |
| | CCCAATGACCCCC | |
| | GGCATA | |
| 112 | CACGCGGCTCCCG | Nucleotide sequences of ARFP- |
| | TCCATCCTGGGGC | derived F101-115; HCV subtype 3a |
| | CCAAATGACCCCC | |
| | GGCGGA | |
| 113 | CCCGTGGCTCTCG | Nucleotide sequences of ARFP- |
| | ACCGTCTTGGGGC | derived F101-115; HCV subtype 4a |
| | CCAAATGATCCCC | |
| | GCGGGA | |
| 114 | CCCGAAGCTCTCG | Nucleotide sequences of ARFP- |
| | GCCTAATTGGGGC | derived F101-115; HCV subtype 5a |
| | CCCAATGACCCCC | |
| | GGCGGA | |
| 115 | CCCGCGGCTCCCG | Nucleotide sequences of ARFP- |
| | GCCACACTGGGGC | derived F101-115; HCV subtype 6a |
| | CCCAATGACCCCC | |
| | GGCGTC | |

| | |
|---|---|
| 116- | See Tables 17 and 18 |
| 128 | |

*GenBank accession numbers are in square brackets
**Based on overlap of peptides F29-43, F31-40 and F37-51 (SEQ ID Nos: 14, 15 and 21, respectively)

Nucleotide sequence of HCV F protein derived from GenBank Accession M62321 (484 NT; SEQ ID NO: 7). Asterisks indicate where insertion of two nucleotides would result in a frame shift. Sequence without this insertion may still produce F protein as a result of a translational mechanism:

ATGAGCACGAATCCTAAACCTCAAAAAAAA**AACAAACGTAACACCAAC
CGTCGCCCACAGGACGTCAAGTTCCCGGGTGGCGGTCAGATCGTTGGTGG
AGTTTACTTGTTGCCGCGCAGGGGCCCTAGATTGGGTGTGCGCGCGACGA
GAAAGACTTCCGAGCGGTCGCAACCTCGAGGTAGACGTCAGCCTATCCCC
AAGGCTCGTCGGCCCGAGGGCAGGACCTGGGCTCAGCCCGGGTACCCTTG
GCCCCTCTATGGCAATGAGGGCTGCGGGTGGGCGGGATGGCTCCTGTCTC
CCCGTGGCTCTCGGCCTAGCTGGGGCCCCACAGACCCCCGGCGTAGGTCG
CGCAATTTGGGTAAGGTCATCGATACCCTTACGTGCGGCTTCGCCGACCT
CATGGGGTACATACCGCTCGTCGGCGCCCCTCTTGGAGGCGCTGCCAGGG
CCCTGGCGCATGGCGTCCGGGTTCTGGAAGACGGCG
Amino acid sequence of HCV F protein derived from
GenBank Accession M62321; based on the assumption
of a frameshift at codon 11 of the sequence of
M62321 (162 AA; SEQ ID NO: 8):
MSTNPKPQKKKTNVTPTVAHRTSSSRVAVRSLVEFTCCRAGALDWVCARR
ERLPSGRNLEVDVSLSPRLVGPRAGPGLSPGTLGPSMAMRAAGGRDGSCL
PVALGLAGAPQTPGVGRAIWVRSSIPLRAASPTSWGTYRSSAPLLEALPG
PWRMASGFWKTA
Δ11 truncated version of SEQ ID NO: 8 (151AA; SEQ
ID NO: 9):
TNVTPTVAHRTSSSRVAVRSLVEFTCCRAGALDWVCARRERLPSGRNLEV
DVSLSPRLVGPRAGPGLSPGTLGPSMAMRAAGGRDGSCLPVALGLAGAPQ
TPGVGRAIWVRSSIPLRAASPTSWGTYRSSAPLLEALPGPWRMASGFWKT
A
Nucleotide sequence of Fmut8 protein identified in
the studies described herein (486 NT; SEQ ID NO:
10):
ATGAGCACGAATCCTAAACCTCAGAGGAAGACCCCAAACGTAACACCAAC
CGTCGCCCACAGGACGTCAAGTTCCCGGGTGGCGGTCAGATCGTTGGTGG
AGTTTACTTGTTGCCGCGCAGGGGCCCTAGATTGGGTGTGCGCGCGACGA
GGAAGACTTCCGAGCGGTCGCAACCTCGAGGTAGACGTCAGCCTATCCCC
AAGGCACGTCGGCCCGAGGGCAGGACCTGGGCTCAGCCCGGGTACCCTTG
GCCCCTCTATGGCAATGAGGGCTGCGGATGGGCGGGATGGCTCCTGTCTC
CCCGTGGCTCTCGGCCTAGTTGGGGCCCCACAGACCCCCGGCGTAGGTCG
CGCAATTTGGGTAAGGTCATCGATACCCTCACGTGCGGCTTCGCCGACCT
CATGGGGTACATACCGCTCGTCGGCGCCCCCCTTGGAGGCGCTGCCAGGG
CCCTGGCGCATGGCGTCCGGGTTCTGGAGGACGGCG
Amino acid sequence of Fmut8 protein identified in
the studies described herein (162 AA; SEQ ID NO:
11):
MSTNPKPQRKTPNVTPTVAHRTSSSRVAVRSLVEFTCCRAGALDWVCARR
GRLPSGRNLEVDVSLSPRHVGPRAGPGLSPGTLGPSMAMRAADGRDGSCL
PVALGLVGAPQTPGVGRAIWVRSSIPSRAASPTSWGTYRSSAPPLEALPG
PWRMASGFWRTA
Amino acid sequence of Fmut8Δ11 protein identified
in the studies described herein (151 AA; SEQ ID
NO: 12):
PNVTPTVAHRTSSSRVAVRSLVEFTCCRAGALDWVCARRGRLPSGRNLEV
DVSLSPRHVGPRAGPGLSPGTLGPSMAMRAADGRDGSCLPVALGLVGAPQ
TPGVGRAIWVRSSIPSRAASPTSWGTYRSSAPPLEALPGPWRMASGFWRT
A

TABLE 11
Various HCV F protein-derived immunogenic peptides/epitopes described herein

| | | | | | | | | | |
|---|---|---|---|---|---|---|---|---|---|
| Source* | F29-40 | F29-43 | F29-51 | F31-40 | F31-43 | F31-51 | F37-51 | F101-115 | F107-115 |

| | | | | | | | | | |
|---|---|---|---|---|---|---|---|---|---|
| HCV | VRSLVEFTC | VRSLVEFTCC | VRSLVEFT | SLVEFTCCRA | SLVEFTCC | SLVEFTCCR | CCRAGALDW | PVALGLAGAPQ | AGAPQTPGV |
| subtype | CRA | RAGAL | CCRAGALD | (SEQ ID | RAGAL | AGALDWVCA | VCARRE | TPGV | (SEQ ID |
| 1a | (SEQ ID | (SEQ ID | WVCARRE | NO:15) | (SEQ ID | RRE | (SEQ ID | (SEQ ID | NO:32 |
| [M62321] | NO:51) | NO:14) | (SEQ ID | | NO:53) | (SEQ ID | NO:21) | NO:29) | |
| | | | NO:52) | | | NO:54) | | | |
| HCV | ARSLAEYTC | ARSLAEYTCC | ARSLAEYT | SLAEYTCCRA | SLAEYTCC | SALEYTCCR | CCRAGAPGW | PVALGLAGAPQ | AGAPQTPGV |
| subtype | CRA | RAGAP | CCRAGAPG | (SEQ ID | RAGAP | AGAPGWVCA | VCARQG | TPGV | (SEQ ID |
| 2a | (SEQ ID | (SEQ ID | WVCARQG | NO:16) | (SEQ ID | RQG | (SEQ ID | (SEQ ID | NO:62) |
| [D00944] | NO:55) | NO:56) | (SEQ ID | | NO:58) | (SEQ ID | NO:60) | NO:61) | |
| | | | NO:57) | | | NO:59) | | | |
| HCV | DRSLVEYTC | DRSLVEYTCC | DRSLVEYT | SLVEYTCCRA | SLVEYTCC | SLVEYTCCR | CCRAGAHDW | HAAPVHPGAQM | PGAQMTPGG |
| subtype | CRA | RAGAH | CCRAGAHD | (SEQ ID | RA | AGAHDWVCA | VCARRV | TPGG | (SEQ ID |
| 3a | (SEQ ID | (SEQ ID | WVCARRV | NO:17) | (SEQ ID | RRV | (SEQ ID | (SEQ ID | NO:70) |
| [D17763] | NO:63) | NO:64) | (SEQ ID | | NO:66) | (SEQ ID | NO:68) | NO:69) | |
| | | | NO:65) | | | NO:67) | | | |
| HCV | ARSLAEFTC | ARSLAEFTCC | ARSLAEFT | SLAEFTCCRA | SLAEFTCC | SLAEFTCCR | CCRAGAPDW | PVALDPLGAQM | LGAQMIPAG |
| subtype | CRA | RAGAP | CCRAGAPD | (SEQ ID | RA | AGAPDWVSA | VSARLG | IPAG | (SEQ ID |
| 4a | (SEQ ID | (SEQ ID | WVSARLG | NO:18) | (SEQ ID | RLG | (SEQ ID | (SEQ ID | NO:78) |
| [Y11604] | NO:71) | NO:72) | (SEQ ID | | NO:74) | (SEQ ID | NO:76) | NO:77) | |
| | | | NO:73) | | | NO:75) | | | |
| HCV | VRSLVEFTC | VRSLVEFTCC | VRSLVEFT | SLVEETCCRA | SLVEFTCC | SLVEFTCCR | CCRAGALNW | PEALGLIGAPM | IGAPMTPGG |
| subtype | CRA | RAGAL | CCRAGALN | (SEQ ID | RA | AGALNWVSA | VSARLG | TPGG | (SEQ ID |
| 5a | (SEQ ID | (SEQ ID | WVSARLG | NO:19) | (SEQ ID | RLG | (SEQ ID | (SEQ ID | NO:86) |
| [Y13184] | NO:79) | NO:80) | (SEQ ID | | NO:82) | (SEQ ID | NO:84) | NO:85) | |
| | | | NO:81) | | | NO:83) | | | |
| HCV | VRSLAEFTC | VRSLAEFTCC | VRSLAEFT | SLAEFTCCRA | SLAEFTCC | SLAEFTCCR | CCRARAPGW | PAAPGHTGAPM | TGAPMTPGV |
| subtype | CRA | RARAP | CCRARAPG | (SEQ ID | RA | ARAPGWVCA | VCARRG | TPGV | (SEQ ID |
| 6a | (SEQ ID | (SEQ ID | WVCARRG | NO:20) | (SEQ ID | RRG | (SEQ ID | (SEQ ID | NO:94) |
| [Y12083] | NO:87) | NO:88) | (SEQ ID | | NO:90) | (SEQ ID | NO:92) | NO:93) | |
| | | | NO:89) | | | NO:91) | | | |
| Fmut8 | VRSLVEFTC | VRSLVEFTCC | VRSLVEFT | SLVEFTCCRA | SLVEFTCC | SLVEFTCCR | CCRAGALDW | PVALGLVGAPQ | VGAPQTPGV |
| (described | CRA | RAGAL | CCRAGALD | (SEQ ID | RAGAL | AGALDWVCA | VCARRG | TPGV | (SEQ ID |
| herein; | (SEQ ID | (SEQ ID | WVCARRG | NO:98) | (SEQ ID | RRG | (SEQ ID | (SEQ ID | NO:103) |
| SEQ ID | NO:95) | NO:96) | (SEQ ID | | NO:99) | (SEQ ID | NO:101) | NO:102) | |
| NO:11) | | | NO:97) | | | NO:100) | | | |

*GenBank accession numbers are shown in square brackets

TABLE 12

Nucleotide sequences of ARFP-derived F29-51
peptide in various HCV subtypes.

| HCV | |
|---|---|
| subtype | F29-51 |
| 1a | GTCAGATCGTTGGTGGAGTTTACTTGTTGCCGCGCAGGGGCC |
| (SEQ ID | CTAGATTGGGTGTGCGCGCGACGAGAA |
| NO:104) | |
| 2a | GCCAGATCGTTGGCGGAGTATACTTGTTGCCGCGCAGGGGCC |
| (SEQ ID | CCAGGTTGGGTGTGCGCGCGACAAGGA |
| NO:105) | |
| 3a | GACAGATCGTTGGTGGAGTATACGTGTTGCCGCGCAGGGGCC |
| (SEQ ID | CACGATTGGGTGTGCGCGCGACGCGTA |
| NO:106) | |
| 4a | GCCAGATCGTTGGCGGAGTTTACTTGTTGCCGCGCAGGGGCC |
| (SEQ ID | CCAGATTGGGTGTGCGCGCGACTCGGA |
| NO:107) | |
| 5a | GTCAGATCGTTGGTGGAGTTTACTTGTTGCCGCGCAGGGGCC |
| (SEQ ID | CTAAATTGGGTGTGCGCGCGACTCGGA |
| NO:108) | |
| 6a | GTCAGATCGTTGGCGGAGTTTACTTGTTGCCGCGCAAGGGCC |
| (SEQ ID | CCCGGTTGGGTGTGCGCGCGACGAGGA |
| NO:109) | |

Reference sequences are as per Table 11.

TABLE 13

Nucleotide sequences of ARFP-derived F101-115
peptide in various HCV subtypes.

| HCV | |
|---|---|
| sub- | |
| type | F101-115 |
| 1a | CCCGTGGCTCTCGGCCTAGCTGGGGCCCCACAGACCCCCGGCGTA |
| (SEQ | |
| ID | |
| NO: | |
| 110) | |
| 2a | CCCGAGGTTCCCGTCCCTCTTGGGGCCCCAATGACCCCCGGCATA |
| (SEQ | |
| ID | |
| NO: | |
| 111) | |
| 3a | CACGCGGCTCCCGTCCATCCTGGGGCCCAAATGACCCCCGGCGGA |
| (SEQ | |
| ID | |
| NO: | |
| 112) | |
| 4a | CCCGTGGCTCTCGACCGTCTTGGGGCCCAAATGATCCCCGCGGGA |
| (SEQ | |
| ID | |
| NO: | |
| 113) | |
| 5a | CCCGAAGCTCTCGGCCTAATTGGGGCCCCAATGACCCCCGGCGGA |
| (SEQ | |
| ID | |
| NO: | |
| 114) | |
| 6a | CCCGCGGCTCCCGGCCACACTGGGGCCCCAATGACCCCCGGCGTC |
| (SEQ | |
| ID | |
| NO: | |
| 115) | |

Reference sequences are as per Table 11.

TABLE 14
Various immunogenic peptides described herein derived from
HCV F protein region spanning residues 29-51

| HCV | SEQ | | | | | | | | | | | | | |
|---|---|---|---|---|---|---|---|---|---|---|---|---|---|---|
| sub- | ID | F protein position | | | | | | | | | | | | |
| type | NO: | 29 | 30 | 31 | 32 | 33 | 34 | 35 | 36 | 37 | 38 | 39 | 40 | 41 |
| 1a | | V | R | S | L | V | E | F | T | C | C | R | A | G |
| 2a | | A | R | S | L | A | E | Y | T | C | C | R | A | G |
| 3a | | D | R | S | L | V | E | Y | T | C | C | R | A | G |
| 4a | | A | R | S | L | A | E | F | T | C | C | R | A | G |
| 5a | | V | R | S | L | V | E | F | T | C | C | R | A | G |
| 6a | | V | R | S | L | A | E | F | T | C | C | R | A | R |
| Fmut8* | | V | R | S | L | V | E | F | T | C | C | R | A | G |

| | | | | | | | | | | | | | |
|---|---|---|---|---|---|---|---|---|---|---|---|---|---|
| Formula I | X¹ | X² | X³ | X⁴ | X⁵ | X⁶ | X⁷ | X⁸ | X⁹ | X¹⁰ | X¹¹ | X¹² | X¹³ |

| HCV | SEQ | | | | | | | | | | |
|---|---|---|---|---|---|---|---|---|---|---|---|
| sub- | ID | F protein position | | | | | | | | | |
| type | NO: | 42 | 43 | 44 | 45 | 46 | 47 | 48 | 49 | 50 | 51 |
| 1a | | A | L | D | W | V | C | A | R | R | E |
| 2a | | A | P | G | W | V | C | A | R | Q | G |
| 3a | | A | H | D | W | V | C | A | R | R | V |
| 4a | | A | P | D | W | V | S | A | R | L | G |
| 5a | | A | L | N | W | V | S | A | R | L | G |
| 6a | | A | P | G | W | V | C | A | R | R | G |
| Fmut8* | | A | L | D | W | V | C | A | R | R | G |

| | | | | | | | | | | |
|---|---|---|---|---|---|---|---|---|---|---|
| Formula I | X¹⁴ | X¹⁵ | X¹⁶ | X¹⁷ | X¹⁸ | X¹⁹ | X²⁰ | X²¹ | X²² | X²³ |

*derived from SEQ ID NO: 11

TABLE 15
Various immunogenic peptides described herein derived from
HCV F protein region spanning residues 101-115

| HCV | SEQ | | | | | | | | | | | | | | | |
|---|---|---|---|---|---|---|---|---|---|---|---|---|---|---|---|---|
| sub- | ID | F protein position | | | | | | | | | | | | | | |
| type | NO: | 101 | 102 | 103 | 104 | 105 | 106 | 107 | 108 | 109 | 110 | 111 | 112 | 113 | 114 | 115 |
| 1a | | P | V | A | L | G | L | A | G | A | P | Q | T | P | G | V |
| 2a | | P | E | V | P | V | P | L | G | A | P | M | T | P | G | I |
| 3a | | H | A | A | P | V | H | P | G | A | Q | M | T | P | G | G |
| 4a | | P | V | A | L | D | R | L | G | A | Q | M | I | P | A | G |
| 5a | | P | E | A | L | G | L | I | G | A | P | M | T | P | G | G |
| 6a | | P | A | A | P | G | H | T | G | A | P | M | T | P | G | V |
| Fmut8* | | P | V | A | L | G | L | V | G | A | P | Q | T | P | G | V |

| | | | | | | | | | | | | | | | |
|---|---|---|---|---|---|---|---|---|---|---|---|---|---|---|---|
| Formula II | Z¹ | Z² | Z³ | Z⁴ | Z⁵ | Z⁶ | Z⁷ | Z⁸ | Z⁹ | Z¹⁰ | Z¹¹ | Z¹² | Z¹³ | Z¹⁴ | Z¹⁵ |

*derived from SEQ ID NO: 11

TABLE 16
Start and end positions within HCV F protein* of
various immunogenic peptides/epitopes identified herein

| Start position | End position |
|---|---|
| 29 | 40 |
| 29 | 43 |
| 29 | 51 |
| 29 | 115 |
| 31 | 40 |
| 31 | 43 |
| 31 | 51 |
| 31 | 115 |
| 37 | 51 |
| 37 | 115 |
| 101 | 115 |
| 107 | 115 |

*Examples of HCV F protein include those derived from HCV subtype 1a (GenBank accession M62321), 2a (GenBank accession D00944), 3a (GenBank accession D17763), 4a (GenBank accession Y11604), 5a (GenBank accession Y13184), 6a (GenBank accession Y12083), and Fmut8 (subtype 1a; see SEQ ID NO: 11).

TABLE 17

Nucleotide sequences of ARFP-derived F29-115
peptide region in various HCV subtypes.

| HCV | SEQ | |
|---|---|---|
| sub- | ID | |
| type | NO: | F29-115 |
| 1a | 116 | GTCAGATCGTTGGTGGAGTTTACTTGTTGCCGCGCAGGGGCC |
| | | CTAGATTGGGTGTGCGCGCGACGAGAA |
| | | AGACTTCCGAGCGGTCGCAACCTCGAGGTAGACGTCAGCCTA |
| | | TCCCCAAGGCTCGTCGGCCCGAGGGCA |
| | | GGACCTGGGCTCAGCCCGGGTACCCTTGGCCCCTCTATGGCA |
| | | ATGAGGGCTGCGGGTGGGCGGGATGGC |
| | | TCCTGTCTCCCCGTGGCTCTCGGCCTAGCTGGGGCCCCACAG |
| | | ACCCCCGGCGTA |
| 2a | 117 | GCCAGATCGTTGGCGGAGTATACTTGTTGCCGCGCAGGGGCC |
| | | CCAGGTTGGGTGTGCGCGCGACAAGGA |
| | | AGACTTCGGAGCGGTCCCAGCCACGTGGAAGGCGCCAGCCCA |
| | | TCCCTAAGGATCGGCGCTCCACTGGCA |
| | | AATCCTGGGGAAAACCAGGATACCCCTGGCCCCTATACGGGA |
| | | ATGAGGGACTCGGCTGGGCAGGATGGC |
| | | TCCTGTCCCCCCGAGGTTCCCGTCCCTCTTGGGGCCCCAATG |
| | | ACCCCCGGCATA |
| 3a | 118 | GACAGATCGTTGGTGGAGTATACGTGTTGCCGCGCAGGGGCC |
| | | CACGATTGGGTGTGCGCGCGACGCGTA |
| | | AAACTTCTGAACGGTCACAGCCTCGCGGACGACGACAGCCTA |
| | | TCCCCAAGGCGCGTCGGAGCGAAGGCC |
| | | GGTCCTGGGCTCAGCCCGGGTACCCTTGGCCCCTCTATGGTA |
| | | ACGAGGGCTGCGGGTGGGCAGGGTGGC |
| | | TCCTGTCCCCACGCGGCTCCCGTCCATCCTGGGGCCCAAATG |
| | | ACCCCCGGCGGA |
| 4a | 119 | GCCAGATCGTTGGCGGAGTTTACTTGTTGCCGCGCAGGGGCC |
| | | CCAGATTGGGTGTGCGCGCGACTCGGA |
| | | AGACTTCGGAGCGGTCGCAACCTCGTGGAAGACGCCAACCTA |
| | | TCCCCAAGGCGCGTCGACCCGAGGGAA |
| | | GGTCCTGGGCACAACCAGGATATCCATGGCCTCTTTACGGTA |
| | | AGAGGGTTGTGGGTGGGCAGGATGGC |
| | | TCTTGTCCCCCCGTGGCTCTCGACCGTCTTGGGGCCCAAATG |
| | | ATCCCCGCGGGA |
| 5a | 120 | GTCAGATCGTTGGTGGAGTTTACTTGTTGCCGCGCAGGGGCC |
| | | CTAAATTGGGTGTGCGCGCGACTCGGA |
| | | AGAATTCGGAACGGTCGCAACCCCGTGGACGGCGCCAGCCTA |
| | | TTCCCAAGGCGCGCCGACCCACGGGCC |
| | | GGTCCTGGGGTCAACCCGGGTACCCTTGGCCCCTTTACGCCA |
| | | ATGAAGGCCTCGGGTGGGCAGGGTGGT |
| | | TGCTCTCCCCCCGAAGCTCTCGGCCTAATTGGGGCCCCAATG |
| | | ACCCCCGGCGGA |
| 6a | 121 | GTCAGATCGTTGGCGGAGTTTACTTGTTGCCGCGCAAGGGCC |
| | | CCCGGTTGGGTGTGCGCGCGACGAGGA |
| | | AGACTTCTGAGCGATCCCAGCCCAGAGGCAGGCGCCAACCTA |
| | | TACCAAAGGCGCGCCAGCCCCAGGGCA |
| | | GGCACTGGGCTCAGCCCGGATACCCTTGGCCTCTTTATGGAA |
| | | GCGAAGGCTGTGGGTGGGCAGGTTGGC |
| | | TCCTGTCCCCCCGCGGCTCCCGGCCACACTGGGGCCCCAATG |
| | | ACCCCCGGCGTC |

Sequences derived from GenBank accession Nos. according to subtype as per Table 3.

TABLE 18

Amino acid sequences of ARFP-derived F29-115
peptide region in various HCV subtypes.

| HCV | SEQ | |
|---|---|---|
| sub- | ID | |
| type | NO: | F29-115 |
| 1a | 122 | VRSLVEFTCCRAGALDWVCARRERLPSGRNLEVDVSLSPRL |
| | | VGPRAGPGLSPGTLGPSMAMRAAGGRDGSCLPVALGLAGAPQTPGV |
| 2a | 123 | ARSLAEYTCCRAGAPGWVCARQGRLRSGPSHVEGASPSLRI |
| | | GAPLANPGENQDTPGPYTGMRDSAGQDGSCPPEVPVPLGAP |
| | | MTPGI |
| 3a | 124 | DRSLVEYTCCRAGAHDWVCARRVKLLNGHSLADDDSLSPRR |
| | | VGAKAGPGLSPGTLGPSMVTRAAGGQGGSCPHAAPVHPGAQ |
| | | MTPGG |
| 4a | 125 | ARSLAEFTCCRAGAPDWVCARLGRLRSGRNLVEDANLSPRR |
| | | VDPREGPGHNQDIHGLFTVMRVVGGQDGSCPPVALDRLGAQ |
| | | MIPAG |
| 5a | 126 | VRSLVEFTCCRAGALNWVCARLGRIRNGRNPVDGASLFPRR |
| | | ADPRAGPGVNPGTLGPFTPMKASGGQGGCSPPEALGLIGAP |
| | | MTPGG |
| 6a | 127 | VRSLAEFTCCRARAPGWVCARRGRLLSDPSPEAGANLYQRR |
| | | ASPRAGTGLSPDTLGLFMEAKAVGGQVGSCPPAAPGHTGAP |
| | | MTPGV |
| Fmut8 | 128 | VRSLVEFTCCRAGALDWVCARRGRLPSGRNLEVDVSLSPRH |
| | | VGPRAGPGLSPGTLGPSMAMRAADGRDGSCLPVALGLVGAP |
| | | QTPGV |

Sequences derived from GenBank accession Nos. according to subtype as per Table 3; Fmut8 sequences derived from SEQ ID NO: 11.

Throughout this application, various references are referred to describe more fully the state of the art to which this invention pertains. The disclosures of these references are hereby incorporated by reference into the present disclosure.

### REFERENCES

1. Battegay M, Fikes J, Di Bisceglie A M, Wentworth P A, Sette A, Celis E, et al. Patients with chronic hepatitis C have circulating cytotoxic T cells which recognize hepatitis C virus-encoded peptides binding to HLA-A2.1 molecules. J Virol 1995; 69:2462-2470.
2. Rehermann B, Chang K M, McHutchison J G, Kokka R, Houghton M, Chisari F V. Quantitative analysis of the peripheral blood cytotoxic T lymphocyte response in patients with chronic hepatitis C virus infection. J Clin Invest 1996; 98:1432-1440.
3. Cooper S, Erickson A L, Adams E J, Kansopon J, Weiner A J, Chien D Y, et al. Analysis of a successful immune response against hepatitis C virus. Immunity 1999; 10:439-449.
4. Lechner F, Wong D K H, Dunbar P R, Chapman R, Chung R T, Dohrenwend P, et al. J Exp Med 2000; 191:1499-1512.
5. Ockenga J, Tillmann H L, Trautwein C, Stoll M, Manns M P, Schmidt R E. Hepatitis B and C in HIV-infected patients. Prevalence and prognostic value. J Hepatol 1997; 27:18-24.
6. Sulkowski M S, Mast E E, Seef L B, Thomas D L. Hepatitis C virus infection as an opportunistic disease in persons infected with human immunodeficiency virus. Clin Infect Dis 2000; 30(S1):S77-S84.
7. Cribier B, Rey D, Schmitt C, Lang J M, Kirn A, Stoll-Keller F. High hepatitis C viraemia and impaired antibody response in patients coinfected with HIV. AIDS 1995; 9:1131-1136.
8. George S L, Gebhardt J, Klinzman D, Foster M B, Patrick K D, Schmidt W N, et al. Hepatitis C virus viremia in HIV-infected individuals with negative HCV antibody tests. J Acquir Immune Defic Syndr 2002; 31:154-162.
9. Lauer G M, Nguyen T N, Day C L, Robbins G K, Flynn T, McGowan K, et al. Human immunodeficiency virus type 1-hepatitis C virus coinfection: intraindividual comparison of cellular immune responses against two persistent viruses. J Virol 2002; 76:2817-2826.
10. Elliott T, Bodmer H, Townsend A. Recognition of out-of-frame major histocompatibility complex class I-restricted epitopes in vivo. Eur J Immunol 1996; 26:1175-1179.
11. Ronsin C, Chung-Scott V, Poullion I, Aknouche N, Gaudin C, Triebel F. A non-AUG-defined alternative open reading frame of the intestinal carboxyl esterase mRNA generates an epitope recognized by renal cell carcinoma-reactive tumor-infiltrating lymphocytes in situ. J Immunol 1999; 163:483-490.
12. Smith D B, Simmonds P. Characteristics of nucleotide substitution in the hepatitis C virus genome: constraints on sequence change in coding regions at both ends of the genome. J Mol Evol 1997; 45:238-246.
13. Xu Z, Choi J, Yen T S, Lu W, Strohecker A, Govindarajan A, et al. Synthesis of a novel hepatitis C virus protein by ribosomal frameshift. EMBO J. 2001; 20:3840-3848.
14. Walewski J L, Keller T R, Stump D D, Branch A D. Evidence for a new hepatitis C virus antigen encoded in an overlapping reading frame. RNA 2001; 7:710-721.
15. Lo S Y, Selby M, Tong M, Ou J H. Comparative studies of the core gene products of two different hepatitis C virus isolates: two alternative forms determined by a single amino-acid substitution. Virology 1994; 199:124-131.
16. Bain C, Parroche P, Layergne J P, Duverger B, Vieux C, Dubois V, et al. Memory T-cell-mediated immune responses specific to an alternative core protein in hepatitis C virus infection. J Virol 2004; 78:10460-10469.
17. Komurian-Pradel F, Rajoharison A, Berland J L, Khouri V, Perret M, van Roosmalen M, et al. Antigenic relevance of F protein in chronic hepatitis C virus infection. Hepatology 2004; 40:900-909.
18. Murphy D, Willems B, Delage G. Use of the 5′ noncoding region for genotyping hepatitis C virus. J Infect Dis 1994; 169:473-475.
19. Chakrabarti S, Brechling K, Moss B. Vaccinia virus expression vector: coexpression of beta-galactosidase provides visual screening of recombinant virus plaques. Mol Cell Biol 1985; 5:3403-3409.
20. Eng J K, McCormack A L, Yates J R. An approach to correlate tandem mass spectral data of peptides with amino acid sequences in a protein database. J Am Soc Mass Spectrom 1994; 5:976-989.
21. Pantaleo G, Soudeyns H, Demarest J F, Vaccarezza M, Graziosi C, Paolucci S, et al. Evidence for rapid disappearance of initially expanded HIV-specific CD8+ T cell clones during primary infection. Proc Natl Acad Sci USA 1997; 94:9848-9853.
22. Brunner K T, Mauel J, Cerottini J C, Chapuis B. Quantitative assay of the lytic action of immune lymphoid cells on 51-Cr-labelled allogeneic target cells in vitro; inhibition by isoantibody and by drugs. Immunology 1968; 14:181-196.
23. Koziel M J, Dudley D, Afdhal N, Grakoui A, Rice C M, Choo Q L, et al. HLA class I-restricted cytotoxic T lymphocytes specific for hepatitis C virus. Identification of multiple epitopes and characterization of patterns of cytokine release. J Clin Invest 1995; 96:2311-2321.
24. Choo Q L, Kuo G, Weiner A J, Overby L R, Bradley D W, Houghton M. Isolation of a cDNA clone derived from a blood-borne non-A, non-B viral hepatitis. Science 1989; 244:359-362.
25. Crumpacker D B, Alexander J, Cresswell P, Engelhard V H. 1992. Role of endogenous peptides in murine allogeneic cytotoxic T cell responses assessed using transfectants of the antigen-processing mutant 174×CEM.T2. J Immunol 1992; 148:3004-3011.
26. Choi J, Xu Z, Ou J H. Triple decoding of hepatitis C virus RNA by programmed translational frameshifting. Mol Cell Biol 2003; 23:1489-1497.
27. Boulant S, Becchi M, Penin F, Layergne J P. Unusual multiple recoding events leading to alternative forms of hepatitis C virus core protein from genotype 1b. J Biol Chem 2003; 278:45785-45792.
28. He X S, Rehermann B, Lopez-Labrador F X, Boisvert J, Cheung R, Mumm J, et al. Quantitative analysis of hepatitis C virus-specific CD8+ T cells in peripheral blood and liver using peptide-MHC tetramers. Proc Natl Acad Sci USA 1999; 96:5692-5697.
29. Walewski J L, Gutierrez J A, Branch-Elliman W, Stump D D, Keller T R, Rodriguez A, et al. Mutation Master: profiles of substitutions in hepatitis C virus RNA of the core, alternate reading frame, and NS2 coding regions. RNA 2002; 8:557-571.
30. Wang H, Bian T, Merrill S J, Eckels D D. Sequence variation in the gene encoding the nonstructural 3 protein of hepatitis C virus: evidence for immune selection. J Mol Evol 2002; 54:465-473.
31. Weiner A, Erickson A L, Kansopon J. Crawford K, Muchmore E, Hughes A L, et al. Persistent hepatitis C virus infection in a chimpanzee is associated with emergence of a cytotoxic T lymphocyte escape variant. Proc Natl Acad Sci USA 1995; 92:2755-2759.
32. Yewdell J W, Del Val M. Immunodominance in TCD8+ responses to viruses: cell biology, cellular immunology, and mathematical models. Immunity 2004; 21:149-153.
33. Basu A, Steele R, Ray R, Ray R B. Functional properties of a 16 kDa protein translated from an open reading frame of the core-encoding genomic region of hepatitis C virus. J Gen Virol 2004; 85:2299-2306.
34. Parker K C, Bednarek M A, Coligan J E. 1994. Scheme for ranking potential HLA-A2 binding peptides based on independent binding of individual peptide side-chains. J Immunol 152: 163-175.
35. Rammensee H G, Bachmann J, Emmerich N N, Bachor O A, Stevanovic S. 1999. SYFPEITHI: database for MHC ligands and peptide motifs. Immunogenetics 50: 213-219.
36. Bain, C. et al (2004) PCT International Patent Application published (in French) as WO04069864A1 on Aug. 19, 2004.

1. An isolated immunogenic polypeptide, wherein said polypeptide is 50 amino acids or less in length and comprises an epitope corresponding to residue 31 to 40 of an HCV F protein.

2. The isolated polypeptide of claim 1, wherein said HCV F protein is derived from an HCV of a subtype selected from the group consisting of HCV-1a, HCV-2a, HCV-3a, HCV-4-a, HCV-5a and HCV-6a.

3. The isolated polypeptide of claim 1, wherein said HCV F protein is derived from an HCV-1a subtype.

4. The isolated polypeptide of claim 1, wherein said peptide is selected from the group consisting of:
(a) a polypeptide comprising an amino acid sequence selected from the group consisting of SEQ ID NOs: 14 to 20, 51 to 59, 63 to 67, 71 to 75, 79 to 83, 87 to 91 and 95 to 100; and
(b) a functional variant or fragment of (a), wherein said functional variant or fragment has an immune-related activity.

5. The isolated polypeptide of claim 4, wherein said immune-related activity is selected from the group consisting of:
(i) an induction of an immune response against HCV;
(ii) an induction of T-cell lytic activity;
(iii) binding to a human leukocyte antigen (HLA) or MHC class I molecule;
(iv) immunoreactivity with serum from an HCV-infected subject;
(v) an alteration in cytokine or chemokine expression or production; and
(vi) (vi) any combination of (i) to (v).

6. The isolated polypeptide of claim 5, wherein said HLA molecule is an HLA-A molecule.

7. The isolated polypeptide of claim 6, wherein said HLA-A molecule is an HLA-A*0201 molecule.

8. (canceled)

9. The isolated polypeptide of claim 1, wherein said polypeptide consists essentially of an amino acid sequence selected from the group consisting of SEQ ID NOs: 14 to 20, 51 to 59, 63 to 67, 71 to 75, 79 to 83, 87 to 91 and 95 to 100.

10. The polypeptide of claim 1, wherein said polypeptide is recombinant.

11. A preparation comprising the polypeptide of claim 1, wherein said preparation is substantially free of an HCV protein other than the HCV F protein.

12. The preparation of claim 11, wherein said HCV protein other than the HCV F protein is an HCV core protein.

13-19. (canceled)

20. A pharmaceutical composition comprising the isolated polypeptide of claim 1, and a pharmaceutically acceptable carrier.

21. The pharmaceutical composition of claim 20, further comprising an adjuvant.

22-23. (canceled)

24. The pharmaceutical composition of claim 20, further comprising an MHC molecule.

25. A composition comprising a multimer of two or more MHC peptide complex monomers, each of said monomers comprising a polypeptide of claim 1 and an MHC molecule.

26-31. (canceled)

32. An isolated nucleic acid encoding the polypeptide of claim 1.

33-36. (canceled)

37. A vector comprising the nucleic acid of claim 32 operably-linked to a transcriptional regulatory sequence.

38. A host cell transformed or transfected with the vector of claim 37.

39. A method of producing a polypeptide of claim 1, said method comprising culturing the host cell of claim 38 under conditions permitting expression of said polypeptide.

40-42. (canceled)

43. A method of preventing or treating HCV infection, or for inducing an immunological or protective immune response against HCV, in an animal, said method comprising administering to said animal the polypeptide of claim 1.

44. (canceled)

45. The method of claim 43, wherein the animal is a human.

46-50. (canceled)

51. A method of detecting or diagnosing HCV infection in an animal, said method comprising assaying a biological sample of said animal with the polypeptide of claim 1.

52-63. (canceled)

64. A polypeptide microarray comprising the polypeptide of claim 1 bound to a substrate.

65-67. (canceled)

68. A method of detecting or diagnosing HCV infection in an animal, said method comprising:
(a) contacting a biological sample of said animal with the polypeptide microarray of claim 64; and
(b) determining the binding of a constituent of the biological sample to said polypeptide microarray;
wherein said binding is indicative of HCV infection.

69-82. (canceled)


SEQUENCE LISTING (128 sequences)

SEQ ID NO: 1 - DNA, 16 bp, Artificial Sequence
  Synthetic Oligonucleotide
gccagccccctgatgg

SEQ ID NO: 2 - DNA, 19 bp, Artificial Sequence
  Synthetic Oligonucleotide
gcctcgtacacaatactcg

SEQ ID NO: 3 - DNA, 54 bp, Artificial Sequence
  Synthetic Oligonucleotide
gaccgtcgaccatgagcacgaatcctaaacctcagaggaagaccccaaacgtaa

SEQ ID NO: 4 - DNA, 37 bp, Artificial Sequence
  Synthetic Oligonucleotide
aagggtacccgggctgagcccaggtcctgccctcggg

SEQ ID NO: 5 - DNA, 29 bp, Artificial Sequence
  Synthetic Oligonucleotide
tcaagtcgacccaaacgtaacaccaaccg

SEQ ID NO: 6 - DNA, 31 bp, Artificial Sequence
  Synthetic Oligonucleotide
ggaaacagctatgaccatgattacgccaagc

SEQ ID NO: 7 - DNA, 486 bp, Hepatitis C virus
  misc_feature (31)..(31): n is a, c, g, t, u, unknown, other or absent
atgagcacgaatcctaaacctcaaaaaaaannaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcg

SEQ ID NO: 8 - protein, 162 aa, Hepatitis C virus
MSTNPKPQKKKTNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 9 - protein, 151 aa, Hepatitis C virus
TNVTPTVAHRTSSSRVAVRSLVEFTCCRAGALDWVCARRERLPSGRNLEVDVSLSPRLVGPRAGPGLSPGTLGPSMAMRAAGGRDGSCLPVALGLAGAPQTPGVGRAIWVRSSIPLRAASPTSWGTYRSSAPLLEALPGPWRMASGFWKTA

SEQ ID NO: 10 - DNA, 486 bp, Hepatitis C virus
atgagcacgaatcctaaacctcagaggaagaccccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggctgcggatgggcgggatggctcctgtctccccgtggctctcggcctagttggggccccacagacccccggcgtaggtcgcgcaatttgggtaaggtcatcgataccctcacgtgcggcttcgccgacctcatggggtacataccgctcgtcggcgccccccttggaggcgctgccagggccctggcgcatggcgtccgggttctggaggacggcg

SEQ ID NO: 11 - protein, 162 aa, Hepatitis C virus
MSTNPKPQRKTPNVTPTVAHRTSSSRVAVRSLVEFTCCRAGALDWVCARRGRLPSGRNLEVDVSLSPRHVGPRAGPGLSPGTLGPSMAMRAADGRDGSCLPVALGLVGAPQTPGVGRAIWVRSSIPSRAASPTSWGTYRSSAPPLEALPGPWRMASGFWRTA

SEQ ID NO: 12 - protein, 151 aa, Hepatitis C virus
PNVTPTVAHRTSSSRVAVRSLVEFTCCRAGALDWVCARRGRLPSGRNLEVDVSLSPRHVGPRAGPGLSPGTLGPSMAMRAADGRDGSCLPVALGLVGAPQTPGVGRAIWVRSSIPSRAASPTSWGTYRSSAPPLEALPGPWRMASGFWRTA

SEQ ID NO: 13 - protein, 10 aa, Hepatitis C virus
KLVALGINAV

SEQ ID NO: 14 - protein, 15 aa, Hepatitis C virus
VRSLVEFTCCRAGAL

SEQ ID NO: 15 - protein, 10 aa, Hepatitis C virus
SLVEFTCCRA

SEQ ID NO: 16 - protein, 10 aa, Hepatitis C virus
SLAEYTCCRA

SEQ ID NO: 17 - protein, 10 aa, Hepatitis C virus
SLVEYTCCRA

SEQ ID NO: 18 - protein, 10 aa, Hepatitis C virus
SLAEFTCCRA

SEQ ID NO: 19 - protein, 10 aa, Hepatitis C virus
SLVEFTCCRA

SEQ ID NO: 20 - protein, 10 aa, Hepatitis C virus
SLAEFTCCRA

SEQ ID NO: 21 - protein, 15 aa, Hepatitis C virus
CCRAGALDWVCARRE

SEQ ID NO: 22 - protein, 9 aa, Hepatitis C virus
CRAGALDWV

SEQ ID NO: 23 - protein, 10 aa, Hepatitis C virus
CCRAGALDWV

SEQ ID NO: 24 - protein, 9 aa, Hepatitis C virus
CRAGAPGWV

SEQ ID NO: 25 - protein, 9 aa, Hepatitis C virus
CRAGAHDWV

SEQ ID NO: 26 - protein, 9 aa, Hepatitis C virus
CRAGAPDWV

SEQ ID NO: 27 - protein, 9 aa, Hepatitis C virus
CRAGALNWV

SEQ ID NO: 28 - protein, 9 aa, Hepatitis C virus
CRARAPGWV

SEQ ID NO: 29 - protein, 15 aa, Hepatitis C virus
PVALGLAGAPQTPGV

SEQ ID NO: 30 - protein, 10 aa, Hepatitis C virus
ALGLAGAPQT

SEQ ID NO: 31 - protein, 9 aa, Hepatitis C virus
GLAGAPQTP

SEQ ID NO: 32 - protein, 9 aa, Hepatitis C virus
AGAPQTPGV

SEQ ID NO: 33 - protein, 10 aa, Hepatitis C virus
LAGAPQTPGV

SEQ ID NO: 34 - protein, 10 aa, Hepatitis C virus
VPVPLGAPMT

SEQ ID NO: 35 - protein, 10 aa, Hepatitis C virus
APVHPGAQMT

SEQ ID NO: 36 - protein, 10 aa, Hepatitis C virus
ALDRLGAQMI

SEQ ID NO: 37 - protein, 10 aa, Hepatitis C virus
ALGLIGAPMT

SEQ ID NO: 38 - protein, 10 aa, Hepatitis C virus
APGHTGAPMT

SEQ ID NO: 39 - protein, 10 aa, Hepatitis C virus
MSTNPKPQRK

SEQ ID NO: 40 - protein, 13 aa, Hepatitis C virus
VAVRSLVEFTCCR

SEQ ID NO: 41 - protein, 10 aa, Hepatitis C virus
AGALDWVCAR

SEQ ID NO: 42 - protein, 19 aa, Hepatitis C virus
RGRLPSGRNLEVDVSLSPR

SEQ ID NO: 43 - protein, 10 aa, Hepatitis C virus
GRLPSGRNLE

SEQ ID NO: 44 - protein, 13 aa, Hepatitis C virus
VDVSLSPRHVGPR

SEQ ID NO: 45 - protein, 17 aa, Hepatitis C virus
AGPGLSPGTLGPSMAMR

SEQ ID NO: 46 - protein, 22 aa, Hepatitis C virus
DGSCLPVALGLVGAPQTPGVGR

SEQ ID NO: 47 - protein, 11 aa, Hepatitis C virus
AIWVRSSIPSR

SEQ ID NO: 48 - protein, 11 aa, Hepatitis C virus
AASPTSWGTYR

SEQ ID NO: 49 - protein, 25 aa, Hepatitis C virus
AASPTSWGTYRSSAPPLEALPGPWR

SEQ ID NO: 50 - protein, 7 aa, Hepatitis C virus
MASGFWR

SEQ ID NO: 51 - protein, 12 aa, Hepatitis C virus
VRSLVEFTCCRA

SEQ ID NO: 52 - protein, 23 aa, Hepatitis C virus
VRSLVEFTCCRAGALDWVCARRE

SEQ ID NO: 53 - protein, 13 aa, Hepatitis C virus
SLVEFTCCRAGAL

SEQ ID NO: 54 - protein, 21 aa, Hepatitis C virus
SLVEFTCCRAGALDWVCARRE

SEQ ID NO: 55 - protein, 12 aa, Hepatitis C virus
ARSLAEYTCCRA

SEQ ID NO: 56 - protein, 15 aa, Hepatitis C virus
ARSLAEYTCCRAGAP

SEQ ID NO: 57 - protein, 23 aa, Hepatitis C virus
ARSLAEYTCCRAGAPGWVCARQG

SEQ ID NO: 58 - protein, 13 aa, Hepatitis C virus
SLAEYTCCRAGAP

SEQ ID NO: 59 - protein, 21 aa, Hepatitis C virus
SLAEYTCCRAGAPGWVCARQG

SEQ ID NO: 60 - protein, 15 aa, Hepatitis C virus
CCRAGAPGWVCARQG

SEQ ID NO: 61 - protein, 15 aa, Hepatitis C virus
PVALGLAGAPQTPGV

SEQ ID NO: 62 - protein, 9 aa, Hepatitis C virus
AGAPQTPGV

SEQ ID NO: 63 - protein, 12 aa, Hepatitis C virus
DRSLVEYTCCRA

SEQ ID NO: 64 - protein, 15 aa, Hepatitis C virus
DRSLVEYTCCRAGAH

SEQ ID NO: 65 - protein, 23 aa, Hepatitis C virus
DRSLVEYTCCRAGAHDWVCARRV

SEQ ID NO: 66 - protein, 13 aa, Hepatitis C virus
SLVEYTCCRAGAH

SEQ ID NO: 67 - protein, 21 aa, Hepatitis C virus
SLVEYTCCRAGAHDWVCARRV

SEQ ID NO: 68 - protein, 15 aa, Hepatitis C virus
CCRAGAHDWVCARRV

SEQ ID NO: 69 - protein, 15 aa, Hepatitis C virus
HAAPVHPGAQMTPGG

SEQ ID NO: 70 - protein, 9 aa, Hepatitis C virus
PGAQMTPGG

SEQ ID NO: 71 - protein, 12 aa, Hepatitis C virus
ARSLAEFTCCRA

SEQ ID NO: 72 - protein, 15 aa, Hepatitis C virus
ARSLAEFTCCRAGAP

SEQ ID NO: 73 - protein, 23 aa, Hepatitis C virus
ARSLAEFTCCRAGAPDWVSARLG

SEQ ID NO: 74 - protein, 13 aa, Hepatitis C virus
SLAEFTCCRAGAP

SEQ ID NO: 75 - protein, 21 aa, Hepatitis C virus
SLAEFTCCRAGAPDWVSARLG

SEQ ID NO: 76 - protein, 15 aa, Hepatitis C virus
CCRAGAPDWVSARLG

SEQ ID NO: 77 - protein, 15 aa, Hepatitis C virus
PVALDRLGAQMIPAG

SEQ ID NO: 78 - protein, 9 aa, Hepatitis C virus
LGAQMIPAG

SEQ ID NO: 79 - protein, 12 aa, Hepatitis C virus
VRSLVEFTCCRA

SEQ ID NO: 80 - protein, 15 aa, Hepatitis C virus
VRSLVEFTCCRAGAL

SEQ ID NO: 81 - protein, 23 aa, Hepatitis C virus
VRSLVEFTCCRAGALNWVSARLG

SEQ ID NO: 82 - protein, 13 aa, Hepatitis C virus
SLVEFTCCRAGAL

SEQ ID NO: 83 - protein, 21 aa, Hepatitis C virus
SLVEFTCCRAGALNWVSARLG

SEQ ID NO: 84 - protein, 15 aa, Hepatitis C virus
CCRAGALNWVSARLG

SEQ ID NO: 85 - protein, 15 aa, Hepatitis C virus
PEALGLIGAPMTPGG

SEQ ID NO: 86 - protein, 9 aa, Hepatitis C virus
IGAPMTPGG

SEQ ID NO: 87 - protein, 12 aa, Hepatitis C virus
VRSLAEFTCCRA

SEQ ID NO: 88 - protein, 15 aa, Hepatitis C virus
VRSLAEFTCCRARAP

SEQ ID NO: 89 - protein, 23 aa, Hepatitis C virus
VRSLAEFTCCRARAPGWVCARRG

SEQ ID NO: 90 - protein, 13 aa, Hepatitis C virus
SLAEFTCCRARAP

SEQ ID NO: 91 - protein, 21 aa, Hepatitis C virus
SLAEFTCCRARAPGWVCARRG

SEQ ID NO: 92 - protein, 15 aa, Hepatitis C virus
CCRARAPGWVCARRG

SEQ ID NO: 93 - protein, 15 aa, Hepatitis C virus
PAAPGHTGAPMTPGV

SEQ ID NO: 94 - protein, 9 aa, Hepatitis C virus
TGAPMTPGV

SEQ ID NO: 95 - protein, 12 aa, Hepatitis C virus
VRSLVEFTCCRA

SEQ ID NO: 96 - protein, 15 aa, Hepatitis C virus
VRSLVEFTCCRAGAL

SEQ ID NO: 97 - protein, 23 aa, Hepatitis C virus
VRSLVEFTCCRAGALDWVCARRG

SEQ ID NO: 98 - protein, 10 aa, Hepatitis C virus
SLVEFTCCRA

SEQ ID NO: 99 - protein, 13 aa, Hepatitis C virus
SLVEFTCCRAGAL

SEQ ID NO: 100 - protein, 21 aa, Hepatitis C virus
SLVEFTCCRAGALDWVCARRG

SEQ ID NO: 101 - protein, 15 aa, Hepatitis C virus
CCRAGALDWVCARRG

SEQ ID NO: 102 - protein, 15 aa, Hepatitis C virus
PVALGLVGAPQTPGV

SEQ ID NO: 103 - protein, 9 aa, Hepatitis C virus
VGAPQTPGV

SEQ ID NO: 104 - DNA, 69 bp, Hepatitis C virus
gtcagatcgttggtggagtttacttgttgccgcgcaggggccctagattgggtgtgcgcgcgacgagaa

SEQ ID NO: 105 - DNA, 69 bp, Hepatitis C virus
gccagatcgttggcggagtatacttgttgccgcgcaggggccccaggttgggtgtgcgcgcgacaagga

SEQ ID NO: 106 - DNA, 69 bp, Hepatitis C virus
gacagatcgttggtggagtatacgtgttgccgcgcaggggcccacgattgggtgtgcgcgcgacgcgta

SEQ ID NO: 107 - DNA, 69 bp, Hepatitis C virus
gccagatcgttggcggagtttacttgttgccgcgcaggggccccagattgggtgtgcgcgcgactcgga

SEQ ID NO: 108 - DNA, 69 bp, Hepatitis C virus
gtcagatcgttggtggagtttacttgttgccgcgcaggggccctaaattgggtgtgcgcgcgactcgga

SEQ ID NO: 109 - DNA, 69 bp, Hepatitis C virus
gtcagatcgttggcggagtttacttgttgccgcgcaagggcccccggttgggtgtgcgcgcgacgagga

SEQ ID NO: 110 - DNA, 45 bp, Hepatitis C virus
cccgtggctctcggcctagctggggccccacagacccccggcgta

SEQ ID NO: 111 - DNA, 45 bp, Hepatitis C virus
cccgaggttcccgtccctcttggggccccaatgacccccggcata

SEQ ID NO: 112 - DNA, 45 bp, Hepatitis C virus
cacgcggctcccgtccatcctggggcccaaatgacccccggcgga

SEQ ID NO: 113 - DNA, 45 bp, Hepatitis C virus
cccgtggctctcgaccgtcttggggcccaaatgatccccgcggga

SEQ ID NO: 114 - DNA, 45 bp, Hepatitis C virus
cccgaagctctcggcctaattggggccccaatgacccccggcgga

SEQ ID NO: 115 - DNA, 45 bp, Hepatitis C virus
cccgcggctcccggccacactggggccccaatgacccccggcgtc

SEQ ID NO: 116 - DNA, 261 bp, Hepatitis C virus
gtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgta

SEQ ID NO: 117 - DNA, 261 bp, Hepatitis C virus
gccagatcgttggcggagtatacttgttgccgcgcaggggccccaggttgggtgtgcgcgcgacaaggaagacttcggagcggtcccagccacgtggaaggcgccagcccatccctaaggatcggcgctccactggcaaatcctggggaaaaccaggatacccctggcccctatacgggaatgagggactcggctgggcaggatggctcctgtccccccgaggttcccgtccctcttggggccccaatgacccccggcata

SEQ ID NO: 118 - protein, 261 aa, Hepatitis C virus
GACAGATCGTTGGTGGAGTATACGTGTTGCCGCGCAGGGGCCCACGATTGGGTGTGCGCGCGACGCGTAAAACTTCTGAACGGTCACAGCCTCGCGGACGACGACAGCCTATCCCCAAGGCGCGTCGGAGCGAAGGCCGGTCCTGGGCTCAGCCCGGGTACCCTTGGCCCCTCTATGGTAACGAGGGCTGCGGGTGGGCAGGGTGGCTCCTGTCCCCACGCGGCTCCCGTCCATCCTGGGGCCCAAATGACCCCCGGCGGA

SEQ ID NO: 119 - DNA, 261 bp, Hepatitis C virus
gccagatcgttggcggagtttacttgttgccgcgcaggggccccagattgggtgtgcgcgcgactcggaagacttcggagcggtcgcaacctcgtggaagacgccaacctatccccaaggcgcgtcgacccgagggaaggtcctgggcacaaccaggatatccatggcctctttacggtaatgagggttgtgggtgggcaggatggctcttgtccccccgtggctctcgaccgtcttggggcccaaatgatccccgcggga

SEQ ID NO: 120 - DNA, 261 bp, Hepatitis C virus
gtcagatcgttggtggagtttacttgttgccgcgcaggggccctaaattgggtgtgcgcgcgactcggaagaattcggaacggtcgcaaccccgtggacggcgccagcctattcccaaggcgcgccgacccacgggccggtcctggggtcaacccgggtacccttggcccctttacgccaatgaaggcctcgggtgggcagggtggttgctctccccccgaagctctcggcctaattggggccccaatgacccccggcgga

SEQ ID NO: 121 - DNA, 261 bp, Hepatitis C virus
gtcagatcgttggcggagtttacttgttgccgcgcaagggcccccggttgggtgtgcgcgcgacgaggaagacttctgagcgatcccagcccagaggcaggcgccaacctataccaaaggcgcgccagccccagggcaggcactgggctcagcccggatacccttggcctctttatggaagcgaaggctgtgggtgggcaggttggctcctgtccccccgcggctcccggccacactggggccccaatgacccccggcgtc

SEQ ID NO: 122 - protein, 87 aa, Hepatitis C virus
VRSLVEFTCCRAGALDWVCARRERLPSGRNLEVDVSLSPRLVGPRAGPGLSPGTLGPSMAMRAAGGRDGSCLPVALGLAGAPQTPGV

SEQ ID NO: 123 - protein, 87 aa, Hepatitis C virus
ARSLAEYTCCRAGAPGWVCARQGRLRSGPSHVEGASPSLRIGAPLANPGENQDTPGPYTGMRDSAGQDGSCPPEVPVPLGAPMTPGI

SEQ ID NO: 124 - protein, 87 aa, Hepatitis C virus
DRSLVEYTCCRAGAHDWVCARRVKLLNGHSLADDDSLSPRRVGAKAGPGLSPGTLGPSMVTRAAGGQGGSCPHAAPVHPGAQMTPGG

SEQ ID NO: 125 - protein, 87 aa, Hepatitis C virus
ARSLAEFTCCRAGAPDWVCARLGRLRSGRNLVEDANLSPRRVDPREGPGHNQDIHGLFTVMRVVGGQDGSCPPVALDRLGAQMIPAG

SEQ ID NO: 126 - protein, 87 aa, Hepatitis C virus
VRSLVEFTCCRAGALNWVCARLGRIRNGRNPVDGASLFPRRADPRAGPGVNPGTLGPFTPMKASGGQGGCSPPEALGLIGAPMTPGG

SEQ ID NO: 127 - protein, 87 aa, Hepatitis C virus
VRSLAEFTCCRARAPGWVCARRGRLLSDPSPEAGANLYQRRASPRAGTGLSPDTLGLFMEAKAVGGQVGSCPPAAPGHTGAPMTPGV

SEQ ID NO: 128 - protein, 87 aa, Hepatitis C virus
VRSLVEFTCCRAGALDWVCARRGRLPSGRNLEVDVSLSPRHVGPRAGPGLSPGTLGPSMAMRAADGRDGSCLPVALGLVGAPQTPGV